SEQUENCE LISTING (16 sequences)

SEQ ID NO: 1 - RNA, 36 nt, Homo sapiens
  source 1..36: mol_type = unassigned RNA
ttagggttagggttagggttagggttagggttaggg

SEQ ID NO: 2 - DNA, 36 bp, Homo sapiens
  source 1..36: mol_type = unassigned DNA
aatcccaatcccaatcccaatcccaatcccaatccc

SEQ ID NO: 3 - DNA, 60 bp, Homo sapiens
  source 1..60: mol_type = unassigned DNA
ttagggttagggttagggttagggttagggtcagggtcagggtcagggtcagggttaggg

SEQ ID NO: 4 - DNA, 60 bp, Homo sapiens
  source 1..60: mol_type = unassigned DNA
aatcccaatcccagtcccagtcccagtcccagtcccagtcccagtcccagtcccaatccc

SEQ ID NO: 5 - RNA, 55 nt, Homo sapiens
  source 1..55: mol_type = unassigned RNA
ttagggttagggttagggttagggttcagggtcagggtcagggtcagggttaggg

SEQ ID NO: 6 - DNA, 60 bp, Homo sapiens
  source 1..60: mol_type = unassigned DNA
aatcccaatcccaatcccaatcccaatcccagtcccagtcccagtcccagtcccaatccc

SEQ ID NO: 7 - DNA, 36 bp, Homo sapiens
  source 1..36: mol_type = unassigned DNA
ttagggttagggttagggttagggttagggttaggg

SEQ ID NO: 8 - DNA, 60 bp, Homo sapiens
  source 1..60: mol_type = unassigned DNA
aatcccaatcccaatcccaatcccaatcccaaccccaaccccaaccccaaccccaatccc

SEQ ID NO: 9 - RNA, 36 nt, Homo sapiens; identical to SEQ ID NO: 1
  source 1..36: mol_type = unassigned RNA

SEQ ID NO: 10 - DNA, 58 bp, Homo sapiens
  source 1..58: mol_type = unassigned DNA
cccaatcccaatcccaatcccaaatcccaaccccaaccccaaccccaaccccaatccc

SEQ ID NO: 11 - DNA, 36 bp, Homo sapiens
  source 1..36: mol_type = unassigned DNA
aaccccaaccccaaccccaaccccaaccccaacccc

SEQ ID NO: 12 - RNA, 36 nt, Homo sapiens
  source 1..36: mol_type = unassigned RNA
tcagggtcagggtcagggtcagggtcagggtcaggg

SEQ ID NO: 13 - DNA, 36 bp, Homo sapiens
  source 1..36: mol_type = unassigned DNA
tcagggtcagggtcagggtcagggtcagggtcaggg

SEQ ID NO: 14 - DNA, 36 bp, Homo sapiens
  source 1..36: mol_type = unassigned DNA
  modified_base 2: mod_base = i
  modified_base 8: mod_base = i
  modified_base 14: mod_base = i
  modified_base 20: mod_base = i
  modified_base 26: mod_base = i
  modified_base 31: mod_base = i
antcccantcccantcccantcccantcccantccc

SEQ ID NO: 15 - RNA, 26 nt, Homo sapiens
  source 1..26: mol_type = unassigned RNA
cagtagggcggcagaagcggcgtagc

SEQ ID NO: 16 - RNA, 11 nt, Homo sapiens
  source 1..11: mol_type = unassigned RNA
ctcccaggttc

(19) United States
(12) Patent Application Publication
(10) Pub. No.: US 2025/0066864 A1
(43) Pub. Date: Feb. 27, 2025

(54) METHODS FOR IDENTIFYING ADAR1 INHIBITORS, AND COMPOSITIONS AND METHODS OF USE IN TREATING CANCER

(71) Applicant: The Wistar Institute of Anatomy and Biology, Philadelphia, PA (US)

(72) Inventor: Kazuko Nishikura, Haddonfield, NJ (US)

(21) Appl. No.: 18/580,002

(22) PCT Filed: Jul. 20, 2022
(86) PCT No.: PCT/US22/73932

§ 371 Date: Jan. 17, 2024

Related U.S. Application Data
(60) Provisional application No. 63/223,888, filed on Jul. 20, 2021.

(51) Int. Cl. C12Q 1/6897; A61K 31/345; A61K 31/381; A61K 31/415; A61K 31/437; A61K 31/501; A61K 31/506; A61K 31/519; A61K 45/06; A61P 35/00; G01N 33/50

(52) U.S. Cl. CPC C12Q 1/6897; A61K 31/345; A61K 31/381; A61K 31/415; A61K 31/437; A61K 31/501; A61K 31/506; A61K 31/519; A61K 45/06; A61P 35/00; G01N 33/5011; G01N 2333/978

(57) ABSTRACT

Provided herein are methods of screening for ADAR1 inhibitors, and compounds identified by the method. In certain embodiments, the methods include treating with an inhibitor of ADAR1.

### CROSS REFERENCE TO RELATED APPLICATIONS

This is a National Stage Entry under 35 U.S.C. 371 of International Patent Application No. PCT/US2022/073932, filed Jul. 20, 2022, which claims the benefit under 35 USC 119(e) of U.S. Provisional Patent Application No. 63/223,888, filed Jul. 20, 2021. These applications are incorporated by reference herein.

### STATEMENT REGARDING FEDERALLY SPONSORED RESEARCH OR DEVELOPMENT

This invention was made with government support under CA010815 and GM040536 awarded by the National Institutes of Health. The government has certain rights in the invention.

### BACKGROUND

ADAR (Adenosine Deaminase Acting on RNA) converts adenosine residues to inosine (A-to-I RNA editing) specifically in double-stranded RNA (dsRNA). We identified ADAR1, the first member of the ADAR gene family, which led to identification of ADAR2 and ADAR3. Both ADAR1 and ADAR2 are catalytically active enzymes, whereas no catalytic activity of ADAR3 has been shown so far. A-to-I editing occurs most frequently in non-coding regions that contain repetitive elements Alu and LINE, and many millions of editing sites have been identified in the human transcriptome of these repetitive sequences. Research efforts of our laboratory have been focused on understanding the biological functions of ADAR1. Two ADAR1 isoforms, p150 and p110 (FIG. 21), are generated by the use of separate promoters and alternate splicing. ADAR1p150 is mostly in the cytoplasm, whereas ADAR1p110 mainly localizes in the nucleus.

Despite the remarkable clinical successes of immunotherapy with certain cancers such as melanomas, development of resistance to the therapy in many patients is a major problem. Interestingly, CRISPR screening for factors that regulate this resistance to immune checkpoint blockade unexpectedly identified ADAR1p150 as a major regulator. ADAR1p150 regulates the dsRNA sensing mechanism mediated by melanoma-differentiation associated protein 5 (MDA5), mitochondrial antiviral signaling protein (MAVS), and interferon signaling (MDA5-MAVS-IFN signaling). Hyper-editing of 3′UTR Alu dsRNAs by the cytoplasmic ADAR1p150 desensitizes the dsRNA sensing mechanism and consequently lends tumors resistance to immune checkpoint blockade (FIG. 22), revealing the pro-oncogenic nature of ADAR1. Cancers upregulate the expression of ADAR1 and thereby suppress the MDA5-MAVS-IFN pathway and consequently dampen inflammatory responses, which in turn allows them to escape the PD-1 antibody-initiated immuno-surveillance. Two adenosine analogs, 8-azaadenosine and 8-chloroadenosine, have been claimed as effective ADAR inhibitors, and their anti-proliferative effects on certain cancer cells have been reported by several groups. However, a more rigorous study conducted by Jason Weber's group revealed that these adenosine analogs are in fact not ADAR inhibitors, indicating that anti-cancer activities reported previously must be due to their non-specific cytotoxic activities. No effective ADAR1 inhibitor is currently available, and many pharmaceutical companies are racing to identify an effective inhibitor of ADAR1, which would improve the success rate of PD-1 based immunotherapy.

In contrast to the recent advance in knowledge of ADAR1p150 functions, the biological functions of the nuclear-localized ADAR1p110 have remained mostly unknown.

What is needed is ADAR1 inhibitors and methods of screening for the same.

### SUMMARY OF THE INVENTION

Provided herein, in some aspects, are methods of screening for ADAR1 inhibitors. In certain embodiments, the method includes
a) providing a test compound to a cancer cell comprising a nucleic acid that comprises a dual reporter system, said dual reporter system comprising a first reporter, an adenosine-to-inosine (A-to-I) editing site within a stop codon of a short hairpin dsRNA, and a second reporter, wherein the first reporter and second reporter are different, and wherein adenosine to inosine editing alters the stop codon, resulting in in-frame translation of the second reporter, and measuring the level of the first reporter about three days after providing the test compound, wherein a reduction of the level of the first reporter of at least about 70% as compared to a control identifies an inhibitor of ADAR1;
b) providing a test compound to a cancer cell comprising a nucleic acid that comprises a dual reporter system, said dual reporter system comprising a first reporter, an adenosine-to-inosine (A-to-I) editing site within a stop codon of a short hairpin dsRNA, and a second reporter, wherein the first reporter and second reporter are different, and wherein adenosine to inosine editing alters the stop codon, resulting in in-frame translation of the second reporter, and measuring the level of the first reporter and second reporter about 1 day after providing the test compound, wherein the ratio of the second reporter to the first reporter demonstrates A-to-I editing efficiency, wherein a reduction in A-to-I editing efficiency of about 65% or more as compared to a control identifies an inhibitor of ADAR1; and/or
c) providing a test compound to a cancer cell comprising a nucleic acid that comprises a dual reporter system, said dual reporter system comprising a first reporter, an adenosine-to-inosine (A-to-I) editing site within a stop codon of a short hairpin dsRNA, and a second reporter, wherein the first reporter and second reporter are different, and wherein adenosine to inosine editing alters the stop codon, resulting in in-frame translation of the second reporter, and measuring the level of the first reporter about one day after providing the test compound, wherein a reduction of the level of the first reporter of less than about 40% as compared to a control identifies an inhibitor of ADAR1.

In certain embodiments, each of a, b, and c, is performed, and they are performed in any order.

In another aspect, a composition for performing a screen is provided. The composition includes a cancer cell comprising a nucleic acid that comprises a dual reporter system, said dual reporter system comprising i) a first reporter, ii) an adenosine-to-inosine (A-to-I) editing site within a stop codon of a short hairpin dsRNA, and iii) a second reporter, wherein the first reporter and second reporter are different.

In yet another aspect, a pharmaceutical composition comprising a pharmaceutically acceptable carrier and an ADAR1 inhibitor is provided. The ADAR inhibitor includes one or more of:
a) [[amino-[3-chloro-4-[(4-chlorophenyl)methoxy]phenyl]methylidene]amino]5-nitrofuran-2-carboxylate
b) 5,7-dimethyl-2-phenylpyrazolo[1,5-a]pyrimidine
c) 3-chloro-N-(4-cyanophenyl)-1-benzothiophene-2-carboxamide
d) 1-[(4-chlorophenyl)methylsulfanyl]-3-phenylpyrido[1,2-a]benzimidazole-4-carbonitrile
e) N-(1-benzylpiperidin-4-yl)-6-phenylthieno[3,2-d]pyrimidin-4-amine
f) 5-(4-chlorophenyl)-3-methylsulfanyl-1H-pyrazole-4-carbonitrile
g) 4-[6-(3,5-dimethylpyrazol-1-yl)pyridazin-3-yl]thiomorpholine
h) 3-(3,4,5-trimethoxyphenyl)-4,5-dihydro-1H-pyridazin-6-one;
i) 4-(furan-2-yl)-2,6-bis(methylsulfanyl)pyrimidine-5-carbonitrile,
   or a prodrug, derivative, pharmaceutical salt, or analog thereof.

In certain embodiments, the pharmaceutical composition is a combination product, further comprising a checkpoint inhibitor and/or a chemotherapeutic agent.

In another aspect, a method of treating cancer in a subject is provided. The method includes administering a pharmaceutical composition comprising a pharmaceutically acceptable carrier and an ADAR1 inhibitor is provided. The ADAR inhibitor includes one or more of:
a) [[amino-[3-chloro-4-[(4-chlorophenyl)methoxy]phenyl]methylidene]amino]5-nitrofuran-2-carboxylate
b) 5,7-dimethyl-2-phenylpyrazolo[1,5-a]pyrimidine
c) 3-chloro-N-(4-cyanophenyl)-1-benzothiophene-2-carboxamide
d) 1-[(4-chlorophenyl)methylsulfanyl]-3-phenylpyrido[1,2-a]benzimidazole-4-carbonitrile e) N-(1-benzylpiperidin-4-yl)-6-phenylthieno[3,2-d]pyrimidin-4-amine
f) 5-(4-chlorophenyl)-3-methylsulfanyl-1H-pyrazole-4-carbonitrile
g) 4-[6-(3,5-dimethylpyrazol-1-yl)pyridazin-3-yl]thiomorpholine
h) 3-(3,4,5-trimethoxyphenyl)-4,5-dihydro-1H-pyridazin-6-one;
i) 4-(furan-2-yl)-2,6-bis(methylsulfanyl)pyrimidine-5-carbonitrile,
   or a prodrug, derivative, pharmaceutical salt, or analog thereof.

In certain embodiments, the pharmaceutical composition is a combination product, further comprising a checkpoint inhibitor and/or a chemotherapeutic agent.

Other aspects and advantages of the invention will be readily apparent from the following detailed description of the invention.

### BRIEF DESCRIPTION OF THE FIGURES

FIG. 1A-FIG. 1D show ADAR1 depletion resulted in abnormalities of the nucleus and upregulation of DNA damage pathway and cell cycle marker genes. (FIG. 1A) HeLa cells were first transfected with siControl or siADAR1 (siADAR1-1) for 72 h and then treated with CellLight Tubulin-GFP. Nuclei were visualized by staining of DNA with SiR-DNA reagent. Representative images taken from real-time videos are presented. Scale bar, 50 m. (FIG. 1B) The frequency of abnormalities of the nucleus (nucleoplasmic bridge, micronuclei, and multinuclei) was estimated by examining at least 200 individual HeLa cells treated with siControl or siADAR1-1 RNAs. Values are mean±standard error (n=3, biologically independent samples) with significant differences by two-tailed Student's t test indicated, **P<0.01. Scale bar, 5 μm. Values are mean±SD (n=3, biologically independent samples) with significant differences by two-tailed Student's t test indicated, **P<0.01. Scale bar, 10 m. All individual experimental data values and exact P values are presented in Source Data file. (FIG. 1C) Western blotting analysis was done using total cell extracts from HeLa cells treated with siControl or two separate siADAR1 (siADAR1-1 and -2) RNAs for 72 h. Protein molecular weight markers are presented in Source Data file. (FIG. 1D) The R-loop structure consisting of an RNA:DNA hybrid formed between the RNA strand newly transcribed by RNA polymerase II and the template DNA strand with the single-stranded antisense DNA bound with ssDNA-binding protein RPA as well as major regulators are schematically shown.

FIG. 2A-FIG. 2E show A-to-I editing activity of ADAR1p110, not ADAR1p150 or ADAR2, is required for suppression of R-loops. (FIG. 2A-FIG. 2E) Dot blot analysis for RNA:DNA hybrids was conducted using control oligos (FIG. 2A) or genomic DNA (FIG. 2B-FIG. 2E). (FIG. 2A) The S9.6 antibody recognized specifically RNA:DNA but not DNA:DNA or RNA:RNA oligo duplex controls. (FIG. 2B, FIG. 2C) Increased RNA:DNA hybrids were detected only in ADAR1-depleted but not in ADAR2-depleted HeLa cells. (FIG. 2B) The S9.6 antibody signals were abolished by E. coli-RNase H treatment, confirming specific detection of RNA:DNA hybrids. (FIG. 2D) Comparison of RNA:DNA hybrid levels between depletion of ADAR1 versus depletion of known R-loop regulators. (FIG. 2E) Increased RNA:DNA hybrid formation resulting from depletion of endogenous ADAR1 was rescued by infection of ADAR1p110-WT (wild type) but not by infection of ADAR1p110-E912A deamination defective mutant or ADAR1p150-WT. (FIG. 2C-FIG. 2F) Data are mean±SD (n=3, biological replicates); significant differences were identified by two-tailed Student's t tests: *P<0.05, **P<0.01, ***P<0.001, n.s., not significant.

FIG. 3A-FIG. 3C show accumulation of R-loops at telomeres in ADAR1-depleted cells. (FIG. 3A) ADAR1 depletion had no effects on already known sites prone to the formation of R-loops. Six sites were examined by qPCR analysis of DRIP products. Data are mean±SD (n=3, biological replicates); significant differences were identified by two-tailed Student's t tests: n.s., not significant. (FIG. 3B) DRIP products were subjected to genomic DNA dot blot hybridization analysis with a probe containing the G-rich-telomere canonical repeat (TTAGGG), or a probe for α-satellite repeat, Alu, or LINE1 consensus sequence. ADAR1 depletion resulted in increased the formation of RNA:DNA hybrids specifically at telomeric repeats, which was abolished by E. coli-RNase H treatment prior to DRIP. (FIG. 3C) Significance of the increased R-loop formation at telomeric repeats was confirmed by conducting three independent dot blot hybridization analyses of DRIP products. Data are mean±SD (n=3, biological replicates); significant differences were identified by two-tailed Student's t tests: **P<0.01, n.s., not significant.

FIG. 4A-FIG. 4C show ADAR1p110 cannot edit completely matched RNA:DNA hybrids carrying telomeric repeat sequences. (FIG. 4A) Canonical telomeric repeat sequences of G-strand RNA (red) and C-strand DNA (blue) are shown. Six adenosines of the G-strand RNA and twelve adenosines of the C-strand DNA are indicated by numbers 1-6 and 1-12, respectively. (FIG. 4B) In vitro editing assay for completely matched telomeric repeat dsRNA was conducted using ADAR1p110-WT recombinant protein. c In vitro editing assay for completely matched telomeric repeat RNA:DNA hybrids using ADAR1p110-WT recombinant protein. No significant levels of editing for matched RNA:DNA hybrids were detected. (FIG. 4B, FIG. 4C) PCR products (RT-PCR-amplified RNA strands and PCR-amplified DNA strands) were subjected to Sanger sequencing. The editing frequency was estimated as the % ratio of the guanosine (black) peak over the sum of guanosine and adenosine (green) peaks of the sequencing chromatograms.

FIG. 5A-FIG. 5B show detection of telomeric variant repeats in ALT-positive and non-ALT cancer cell lines. (FIG. 5A) Dot blot hybridization analysis for genomic DNA samples was conducted using three separate telomeric repeat probes capable of distinguishing a single-nucleotide mismatch (FIG. 14A-FIG. 14B). In addition to canonical TTAGGG signals, varying amounts of TCAGGG and TTGGGG variant repeat signals were detected in both ALT and non-ALT cancer cell lines, but not in primary human fibroblast cells. (FIG. 5B) Quantitation of canonical and variant telomeric repeats was done by comparing dot blot signals of genomic DNA and canonical and variant repeat-specific control oligos. Three independent dot blot hybridization analyses were performed. Data are mean±SD (n=3, biological replicates).

FIG. 6A-FIG. 6D show increased telomeric RNA:DNA hybrids containing variant repeats in ADAR1-depleted cells. Formation of telomeric repeat RNA:DNA hybrids containing A-C mismatches by in cis slipped hybridization (FIG. 6A, FIG. 6B). (FIG. 6A) TERRA RNAs transcribed from the region containing four TCAGGG (green) variant repeats surrounded by TTAGGG (gray) canonical repeats form an RNA:DNA hybrid containing four C-A mismatches by in cis slipped hybridization to the C-strand DNA containing canonical TTAGGG (CCCTAA) repeats. (FIG. 6B) TERRA RNAs transcribed from the region containing four TTGGGG (orange) variant repeats surrounded by TTAGGG (gray) canonical repeats form an RNA:DNA hybrid containing four A-C mismatches by in cis slipped hybridization to the C-strand DNA containing TTGGGG (CCCCAA) variant repeats. (FIG. 6C, FIG. 6D) Detection of increased RNA:DNA hybrids containing TCAGGG and TTGGGG variant repeats in ADAR1-depleted HeLa cells. (FIG. 6C) DRIP products were examined for G-strand RNAs of UCAGGG variant and UUAGGG canonical repeats by dot blot analysis using high-affinity LNA-oligonucleotide probes capable of distinguishing a single-nucleotide mismatch (FIG. 14C). (FIG. 6D) Similarly, DRIP products were examined for C-strand DNAs of TTAGGG (CCCTAA) canonical and TTGGGG (CCCCAA) variant repeats using LNA-oligonucleotide probes capable of distinguishing a single-nucleotide mismatch (FIG. 14D). (FIG. 6C, FIG. 6D) Dot blot signals were abolished by E. coli-RNase H treatment prior to DRIP. The significance of the increase in RNA:DNA hybrids containing telomeric canonical and variant repeats (RNA and DNA strands) was confirmed by conducting three independent dot blot hybridization analysis of DRIP products. Data are mean±SD (n=3, biological replicates); significant differences were identified by two-tailed Student's t tests: *P<0.05, **P<0.01, ***P<0.001.

FIG. 7A-FIG. 7C show ADAR1p110 edits both RNA and DNA strands of telomeric repeat RNA:DNA hybrids containing A-C mismatches. (FIG. 7A-FIG. 7C) In vitro editing assay for telomeric repeat dsRNA (FIG. 7A) and RNA:DNA hybrids (FIG. 7B, FIG. 7C) containing A-C or C-A mismatches was conducted using ADAR1p110-WT recombinant protein. PCR products (RT-PCR-amplified RNA strands and PCR-amplified DNA strands) were subjected to Sanger sequencing.

FIG. 8A-FIG. 8D show editing of A-C mismatches in telomeric repeat RNA:DNA hybrids facilitates RNase H2 cleavage of RNA strands. (FIG. 8A) F-ADAR1p110-WT recombinant protein was copurified with endogenous RNase H2 subunits H2A and H2C, but not with RNase H1. No RNase H2 association detected with a dsRNA-binding defective mutant F-ADAR1p110-EAA (upper panels). Similarly, FLAG-RNase H2A pulled down endogenous ADAR1p110 (lower panels). Note that both FLAG-AFAR1p110 and FLAG-RNase H2A pulled down endogenous TRF2, a telomere-binding protein and a member of the Shelterin complex, supporting our hypothesis that ADAR1 and RNase H2 collaborate together to resolve telomeric R-loops. Protein molecular weight markers are presented in Source Data file. (FIG. 8B) In vitro assay for digestion of 5′-³²P-labeled RNA strands of telomeric repeat RNA:DNA hybrids by RNase H1 or RNase H2A/2B/2C complexes. RNase H2 could not degrade the RNA strands of telomeric repeat RNA:DNA hybrids containing six A-C mismatches, but began digesting RNA strands of RNA:DNA hybrids containing four A-C mismatches. Replacement of all A-C mismatches with I:C-matched base pairs resulted in efficient digestion of RNA strands by RNase H2. RNase H1 degraded RNA strands regardless of the number of A-C mismatches. (FIG. 8C) Time course of digestion of RNA strands of telomeric repeat RNA:DNA hybrids with varying numbers of A-C mismatches by RNase H1 or RNase H2. Data are mean±SD (n=4, technical replicates); significant differences were identified by two-tailed Student's t tests: *P<0.05, ***P<0.001, n.s., not significant. (FIG. 8D) A-to-I editing of A-C mismatches to I:C base pairs in RNA:DNA hybrids facilitates digestion of RNA strands by RNase H2.

FIG. 9A-FIG. 9B show ADAR1 regulates accumulation of telomeric R-loops only in non-ALT cells. (FIG. 9A) Detection of increased RNA:DNA hybrids only in non-ALT cells. Genomic DNA samples (0.25 g) collected from various cells treated with siControl or siADAR1-1 RNAs for 72 h were examined by dot blot assay using the S9.6 antibody. Data are mean±SD (n=3, biological replicates); significant differences were identified by two-tailed Student's t tests: *P<0.05, **P<0.01, ***P<0.001, n.s., not significant. (FIG. 9B) DRIP products from select cell lines were further subjected to dot blot analysis for the C-strand telomeric repeat DNA using the canonical telomeric repeat (TTAGGG) specific probe (FIG. 14A). Telomeric repeat RNA:DNA hybrids were detected only in HEK293T and HCT116 non-ALT cancer cell lines. Data are mean±SD (n=3, biological replicates); significant differences were identified by two-tailed Student's t tests: *P<0.05, **P<0.01, n.s., not significant. (FIG. 9A, FIG. 9B) All individual experimental data values and exact P values are presented in Source Data file.

FIG. 10A, FIG. 10B, and FIG. 11 show M-phase-specific interaction of ADAR1p110 and RNase H2 in non-ALT cancer cells. (FIG. 10A) Elevated ADAR1 and RNase H2A expression levels detected in non-ALT cells. Western blotting analysis was performed using total cell extract proteins and specific antibodies. (FIG. 10B) Interaction between ADAR1p110 and RNase H2 detected only in non-ALT cancer cells. FLAG-ADAR1p110-WT recombinant protein was copurified with endogenous RNase H2 subunit H2A only from non-ALT cell lines. (FIG. 11) Elevated RNase H2A expression levels and also increased interaction of ADAR1p110 with RNase H2A detected specifically at M phase. FLAG-ADAR1p110-WT pull-down experiments were conducted using HEK293T cells synchronized at M phase using the thymidine-nocodazole double block system.

FIG. 12A-FIG. 12C show validation of siRNA gene knockdown and confirmation of FLAG-ADAR1 expression levels. (FIG. 12A) ADAR1 and ADAR2 knockdown efficiency by each siRNA was confirmed by immunoblotting. b-actin was used as a loading control. (FIG. 12B) Lentiviruses for empty vector control or ectopic expression of F-ADAR1p110-WT, F-ADAR1p110-E912A, or F-ADAR1p150-WT were infected in HeLa cells that had been transfected with synthetic siRNAs corresponding to the human ADAR1 mRNA 3′UTR sequence. The expression levels of exogenous FLAG-ADAR1 protein were examined by western blotting analysis using anti-FLAG M2 and anti-ADAR1 antibodies. (FIG. 12C) R-loop regulator knockdown efficiency by each siRNA was confirmed by immunoblotting. b-actin was used as a loading control. Asterisks show nonspecific bands (RNase H2A panel).

FIG. 13 shows validation of siRNA gene knockdown and confirmation of FLAG-ADAR1 expression levels. ADAR1 siRNA-mediated knockdown in each cell line was confirmed by immunoblotting (continued from FIG. 12).

FIG. 14A-FIG. 14D show validation of telomeric probe specificity. (FIG. 14A) Specific hybridization of telomeric probes was confirmed using control oligo DNAs. The canonical TTAGGG probes hybridized specifically to CCCTAA oligos but not to CCCTGA or CCCCAA oligos. TCAGGG and TTGGGG variant repeat probes also specifically bound to CCCTGA or CCCCAA oligos, respectively. (FIG. 14B) Control oligo DNAs were quantitatively detected by the canonical TTAGGG and variant CCCCAA probes. The signals of TTAGGG probes against (CCCTAA)₂-CCCCAA oligos were higher than those of CCCTAA-(CCCCAA)₂ oligos, whereas the TTGGGG probe signals of (CCCTAA)₂-CCCCAA oligos were lower than those of CCCTAA-(CCCCAA)₂ oligos. (CCCCCC)₄, and (CCCCAC)₆ or (CCCCAC)₄ oligos were not detected by both TTAGGG canonical and TTGGGG variant probes. Specific hybridization of locked telomeric repeat probes was confirmed using control RNA (FIG. 14C) or DNA (FIG. 14D) oligos. (FIG. 14C) The LNA canonical CCCTAA and variant CCCTGA probe hybridized specifically to UUAGGG and UCAGGG RNA oligos, respectively. (FIG. 14D) The LNA canonical TTAGGG and variant TTGGGG probe hybridized specifically to CCCTAA and CCCCAA DNA oligos, respectively.

FIG. 15A-FIG. 15B show formation of telomeric repeat RNA:DNA hybrids containing C-A and A-C mismatches by in trans hybridization. (FIG. 15A) In trans hybridization of TERRA RNAs derived from TCAGGG (UCAGGG) variant repeats (green) to the C-strand DNA containing TTAGGG (CCCTAA) canonical repeats (gray), or (FIG. 15B) TERRA RNAs derived from TTAGGG (UUAGGG) canonical repeats (gray) to the C-strand DNA containing TTGGGG (CCCCAA) variant repeats (orange) results in formation of RNA:DNA hybrids containing C-A and A-C mismatches, respectively.

FIG. 16 shows ADAR1 dsRNA binding domains are required for editing of RNA:DNA hybrids. No editing of RNA:DNA hybrids detected with ADAR1p110-EAA dsRNA binding defective mutant. In vitro editing assay was conducted using HA-ADAR1p110-EAA. PCR products (RT-PCR amplified RNA strands and PCR amplified DNA strands) were subjected to Sanger sequencing. The strands analyzed by sequencing are indicated by red and blue arrowheads. ADAR1p110-EAA did not display any editing activity for any of the substrates tested.

FIG. 17 shows no interaction between ADAR2 and RNase H1 or RNase H2A and H2C subunits was detected. FLAG-tagged ADAR2 (F-ADAR2-WT and F-ADAR2-EAA) recombinant proteins were purified from permanently transfected HEK293T cell extracts by anti-FLAG M2 magnetic beads. RNase H2 subunits 2A and 2C or RNase H1 were not pulled down with either F-ADAR2-WT or F-ADAR2-EAA.

FIG. 18 shows expansion model of TCAGGG variant repeat by A-to-I editing. A-to-I editing of C-A mismatches to C:I base pairs in RNA:DNA hybrids formed between variant and canonical repeats facilitated digestion of RNA strands by RNase H2A/2B/2C triple complexes. Replication of A-to-I edited C-strand DNA may further amplify variant TCAGGG repeats.

FIG. 19 shows detection of RNA:DNA hybrids containing telomeric repeats in ADAR1 depleted non-ALT, ALT, and primary fibroblast cells. DRIP products were examined for C-strand DNAs of telomeric canonical TTAGGG (CCCTAA) repeats by dot blot analysis using an oligonucleotide probe capable of distinguishing a single-nucleotide mismatch.

FIG. 20 shows ADAR1 together with RNase H2 resolves telomeric R-loops in non-ALT cancer cells by editing A-C mismatches of RNA:DNA hybrids formed between canonical and variant repeats. Telomeric variant repeats, widespread in both ALT and non-ALT cancer cells, cause formation of RNA:DNA hybrids containing A-C mismatches. Unlike RNase H1, RNase H2 cannot degrade the RNA strands of these mismatch-containing RNA:DNA hybrids. Recruitment of RNase H1, perhaps guided by RPA protein bound to single-stranded G-strand DNAs⁴⁰, and efficient resolution of telomeric R-loops by RNase H1 have been reported in ALT cells⁵². Close and specific interaction of ADAR1p110 with RNase H2, but not RNase H1, is detected only in non-ALT cells. In non-ALT cells, upregulated ADAR1p110 edits these A-C mismatches to I:C matched base pairs, which is essential for removal of the RNA strands by RNase H2. The cell cycle specific (G2-M) function of RNase H2 has been recently reported⁵⁷. Thus, the R-loop regulatory function of ADAR1p110 may be exerted during G2-M phase. In the absence of ADAR1p110, non-ALT cancer cells die due to genome instability caused by accumulation of telomeric R-loops and mitotic arrest.

FIG. 21 shows two ADAR1 isoforms p150 and p110. ADAR1 contains Z DNA binding domains, dsRNA binding domains (dsRBDs), and a separate deaminase domain. There are two isoforms of ADAR1, a full-length interferon-inducible p150 and a shorter N-terminus truncated p110, which are generated through usage of different promoters and alternative splicing. Although both isoforms are catalytically active enzymes, their localization within the cell are very different. ADAR1p150 mainly localizes in cytoplasm due to a strong nuclear export signal present in the first Z DNA, whereas ADAR1p110 is detected mainly in the nucleoplasm and nucleoli.

FIG. 22 shows ADAR1p150 suppresses cancer responsiveness to immune checkpoint blockade by hyper-editing 3′UTRAlu dsRNAs.ADAR1p150. Long dsRNAs are potent inducers of the dsRNA sensing mechanism mediated by MDA5-MAVS-IFN signaling. Long Alu dsRNAs present in 3′UTRs of certain mRNAs that remain unedited in the absence of cytoplasmic ADAR1p150 have been proposed as endogenous inducers of this pathway. IFNs and inflammatory conditions induced by loss of ADAR1 and dsRNA editing activities play important roles in cancer responsiveness to immune checkpoint blockade (upper panel). Hyper-editing of these Alu dsRNAs by ADAR1p150 in the cytoplasm dampens MDA5-MAVS-IFN signaling and thereby contributes to development of the immunotherapy resistance in cancer patients (bottom panel). ADAR1 inhibitors are expected to potentiate the cancer responsiveness to immunotherapy.

FIG. 23 shows ADAR1p110 together with RNase H2 resolves telomeric R-loops in non-ALT cancer cells by editing A-C mismatches of RNA:DNA hybrids formed between canonical and variant repeats. Telomeric variant repeats cause formation of RNA:DNA hybrids containing A-C mismatches. Unlike RNase H1, RNase H2 cannot degrade the RNA strands of these mismatch-containing RNA:DNA hybrids. Close and specific interaction of ADAR1p110 with RNase H2, but not RNase H1, is detected only in non-ALT cells. In non-ALT cells, upregulated ADAR1p110 edits these A-C mismatches to I:C matched base pairs, which is essential for removal of the RNA strands by RNase H2. The cell cycle specific (G2-M) function of RNase H2 has been recently reported. Thus, the R-loop regulatory function of ADAR1p110 is exerted during G2-M phase. In the absence of ADAR1p110, non-ALT cancer cells die due to genome instability caused by accumulation of telomeric R-loops and mitotic arrest.

FIG. 24A-FIG. 24D show dual luciferase reporter system for measurement of ADAR1 specific A-to-I editing activity and also apoptosis induced in ADAR1 depleted HeLa cells. (FIG. 24A) The dual reporter system was used to monitor ADAR1-mediated A-to-I editing activity. The reporter system contains an A-to-I editing site, downstream of Firefly Luciferase (FFL), at an A-C mismatched base pair within a short hairpin dsRNA. Editing of the adenosine to inosine (equivalent to guanosine) changes an amber stop codon to a tryptophan, resulting in in-frame translation of the downstream Nano Luciferase (Nuc). The ratio of Nuc vs FFL represents efficiency of A-to-I editing of the target site. A reporter HeLa cell line was generated by permanent infection of the reporter Lentivirus. (FIG. 24B) The reporter system is specific for A-to-I editing activity of ADAR1: The reporter activity is reduced only in ADAR1 depleted reporter cells. (FIG. 24C) FFL activity can be used to measure cell viability of reporter HeLa cells. Cytotoxic compounds such as Doxorubicin induces acute apoptosis, which can be detected by reduction of the FFL activity. On the other hand, ADAR1 depletion led to induction of “slow” cell death at 3 to 5 days after siRNA treatment. (FIG. 24D) Induction of slow apoptosis by ADAR1 depletion occurs only in non-ALT (telomerase reactivated) cancer cells due to telomere instability and mitotic arrest. Apoptosis measurement was done using Apo-Tox Glo system 4 days after siRNA treatment.

FIG. 25A-FIG. 25C show identification of ADAR1 specific inhibitors by our own two-step molecular screening strategy. (FIG. 25A) The final two-step screening strategy using the dual luciferase reporter system. Induction of “slow” apoptosis determined as a measure of FFL activity reduction at day 3 was used as the first step screening, resulted in pre-selection of 1,600 compounds. Inhibition of A-to-I editing activity measured as the reduction of Nano Luciferase activity (ratio of Nuc vs FFL) at day 1 was used as the second screening. The absence of extreme and acute cytotoxicity (<40% reduction of FFL at day 1) was also taken into account. (FIG. 25B) The second step screening resulted in identification of 54 candidate compounds. (FIG. 25C) Using our well-established in vitro A-to-I editing assay, 54 preselected compounds were evaluated for their ADAR1 inhibitory effects using recombinant ADAR1p110 and synthetic dsRNA substrates, known to be targeted by both ADAR1 and ADAR2, as described previously. Although ADAR1 does edit A:U base pairs of completely matched dsRNAs, A-C mismatched base pairs are in fact the favored ADAR1 target sites. Therefore, both matched (top) and mismatched (bottom) dsRNA substrates were tested. Note that some compounds (compound #13) preferentially inhibited matched adenosines, whereas the others (compound #52) had more specific inhibitory effects on adenosine at A-C mismatched base pairs. We are currently testing their inhibitory effects on RNA:DNA hybrid substrates.

FIG. 26A-FIG. 26B show determination of IC50 by in vitro editing assay. In vitro editing assay using purified ADAR1p110 protein and two different substrates, matched (left) and mismatched (right) dsRNAs, was conducted in the presence of varying concentrations of compound #1 (FIG. 26A) or #13 (FIG. 26B) in order to estimate IC50 values for these compounds. In vitro editing was carried out using HAT-ADAR1p110 or HAT-ADAR2 recombinant protein (10 nM) and two different dsRNA substrates carrying a part of the CDK13 gene sequence (5 nM) at 37° C. for 2 hrs as described previously (12). Compounds were dissolved in 100% DMSO. The in vitro assay contained DMSO at final concentration of 16%.

FIG. 27 shows killing of cancer cells by ADAR1 inhibitor compounds. HeLa, U2OS, and MRI90 cells were cultured in the presence of ADAR1 inhibitors, 5 μM or 20 μM, for 3 days. Their viability was examined using the Apo Tox-Glo assay system (Promega), which measures plasma membrane penetration of glycyl-phenyl-alanyl-aminofluorocoumarin peptide. Control, 0.3% DMSO. Note that compounds #1, #6, #13, and #27, at 5 μM, significantly reduced viability of specifically HeLa cells but not of U2OS nor NMR90 cells. Data are mean±S.D. (n=3, biological replicates).

FIGS. 28A and 28B demonstrates that compound #13 inhibits in vivo ADAR1 RNA editing activity. (FIG. 28A) A known ADAR1 target site in the TMPO mRNA 3′UTR. (FIG. 28B) In vivo editing inhibitory effects of Compound #13 was investigated by cDNA sequencing of RT-PCR products of TMPO mRNAs. HeLa cells were treated with Compound #13 at 10 μM for 24 hrs.

FIGS. 29A-29D Characterization of Compound #13 effects in cancer cells. (FIG. 29A) Compound #13 at 10 μM selectively reduced viability of HeLa non-ALT cancer cells. Viability was examined using the Apo Tox-Glo assay system (Promega). Control, 0.3% DMSO. (FIG. 29B) Western blotting analysis of HeLa cells treated with Compound #13 (10 μM). Note upregulated γ-H2AX, Histone 3 phosphorylated at Serine 10, and cleaved PARP bands. (FIG. 29C) Dot blot analysis for RNA:DNA hybrids induced in HeLa cells by Compound #13 (10 μM). (FIG. 29D) Activation of the IFN signaling pathway. HSC-4 squamous carcinoma cells were treated with Compound #13 at 10 μM (12 hrs). RNA samples were analyzed by qRT-PCR to monitor expression levels of ISGs.

FIG. 30 demonstrates the proposed mechanism for the ADAR-catalyzed RNA editing reaction. Water bound to E396 activated by Zn undergoes nucleophilic attack of the C6 carbon bearing the —NH2 resulting formation of a tetrahedral intermediate. Collapse of this tetrahedral intermediate results in loss of ammonia and formation of inosine.

FIGS. 31A and 31B demonstrates the binding of Compound #13 to ADAR1p110. Compound #13 binds to ADAR1p110 wildtype (FIG. 31A) but not to mutant E912A (FIG. 31B), suggesting the cyano interacts with the active site glutamic acid.

FIGS. 32A-32C show the docking of Compound #13 into the ADAR2 catalytic center pocket (FIG. 32A) The cyano-phenyl moiety of Compound #13 can coordinate with the active site Zn⁺² atom when modeled into the homologous ADAR2 crystal structure (pdb:5hp3). (FIG. 32B, FIG. 32C) Compound #13 may coordinate with the zinc atom to activate the cyano for nucleophilic attack by glutamate to enhance binding affinity.

FIGS. 33A-33D show the expansion of structure-activity-relationship (SAR) of compound 13, and structure-based design to improve potency.

FIGS. 34A-34C show general synthesis approaches to ADAR1 Inhibitors. (FIG. 34A) Amide coupling to synthesize Compound #13 analogs. (FIG. 34B) Click chemistry to synthesize triazole analogs. (FIG. 34C) Pocket filling analogs.

### DETAILED DESCRIPTION OF THE INVENTION

Provided herein are screening methods, compositions for performing same, and ADAR1 inhibitors optionally identified by the described methods. Also provided are methods of using ADAR1 inhibitors for the treatment of telomerase positive cancer. The ADAR1 inhibitors can be used alone or in combination with other cancer therapies, e.g., a checkpoint inhibitor therapy.

### ADAR1

Adenosine deaminase acting on RNA (ADAR) is the enzyme involved in adenosine-to-inosine RNA editing (A-to-I RNA editing), and three ADAR gene family members (ADAR1, ADAR2, and ADAR3) have been identified in vertebrates. ADARs share common domain structures, such as multiple dsRNA-binding domains (dsRBDs) and a separate catalytic domain. Both ADAR1 (ADAR, DRADA) and ADAR2 (ADARB1) are catalytically active enzymes, whereas no catalytic activity of ADAR3 (ADARB2) has been shown so far. A-to-I editing occurs most frequently in noncoding regions that contain repetitive elements Alu and LINE, and many millions of editing sites have been identified in the human transcriptome of these repetitive sequences.

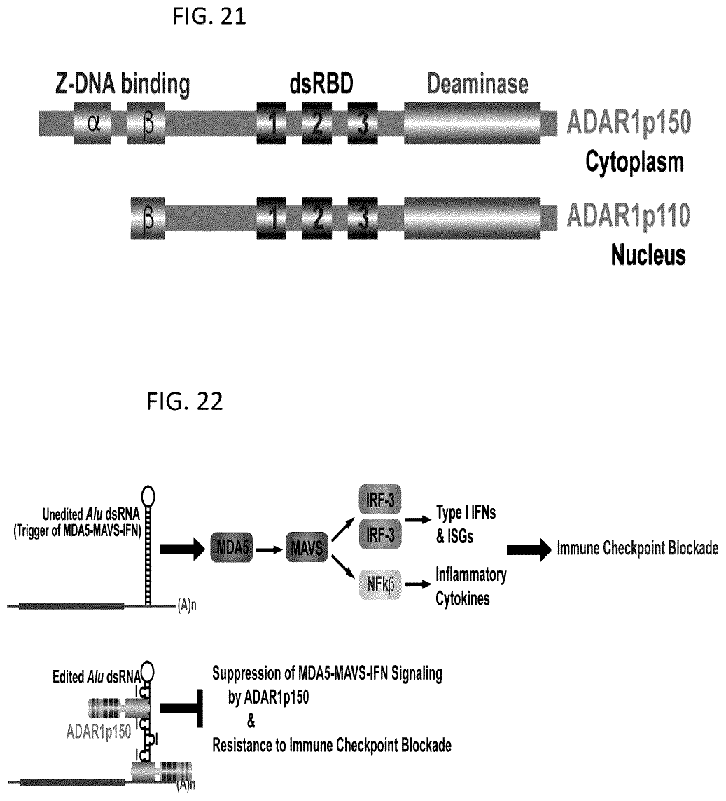

Two ADAR1 isoforms, p150 and p110, are generated by the use of separate promoters and alternate splicing. ADAR1p150 is mostly in the cytoplasm, whereas ADAR1p110 mainly localizes in the nucleus (FIG. 21). The cytoplasmic ADAR1p150 regulates the dsRNA sensing mechanism mediated by melanoma-differentiation associated protein 5 (MDA5), mitochondrial antiviral signaling protein (MAVS), and interferon signaling (MDA5-MAVS-IFN signaling). The cytoplasmic ADAR1p150 edits 3′-untranslated region (3′-UTR) dsRNAs primarily comprising inverted Alu repeats and thereby suppresses activation of MDA5-MAVS-IFN signaling (FIG. 22). This ADAR1p150 function in the regulation of the MDA5-MAVS-IFN pathway underlies the embryonic lethality of Adar1-null mice and also the pathogenesis of Aicardi-Goutieres syndrome (AGS; AGS1-7 subgroups known), a severe human autoimmune disease against endogenous nucleic acids. Mutations of seven genes, including RNaseH2A (AGS4), RNaseH2B (AGS2), RNaseH2C (AGS3), and ADAR1 (AGS6), have been identified in association with AGS, and ten AGS6 mutations of ADAR1 have been reported so far. Finally, this ADAR1p150-mediated suppression of IFN signaling also represses tumor responsiveness to immune checkpoint blockade (FIG. 22), revealing the pro-oncogenic ADAR1p150 function. Analysis of The Cancer Genome Atlas database revealed elevated ADAR1 expression and A-to-I editing levels in almost all types of cancers, indicating that this pro-oncogenic ADAR1p150 function helps cancer cells suppress inflammatory responses and thus avoid host immunosurveillance. In contrast to the recent advance in the knowledge of ADAR1p150 functions, the biological functions of the nuclear-localized ADAR1p110, other than its involvement in editing of intronic Alu dsRNAs, have remained mostly unknown.

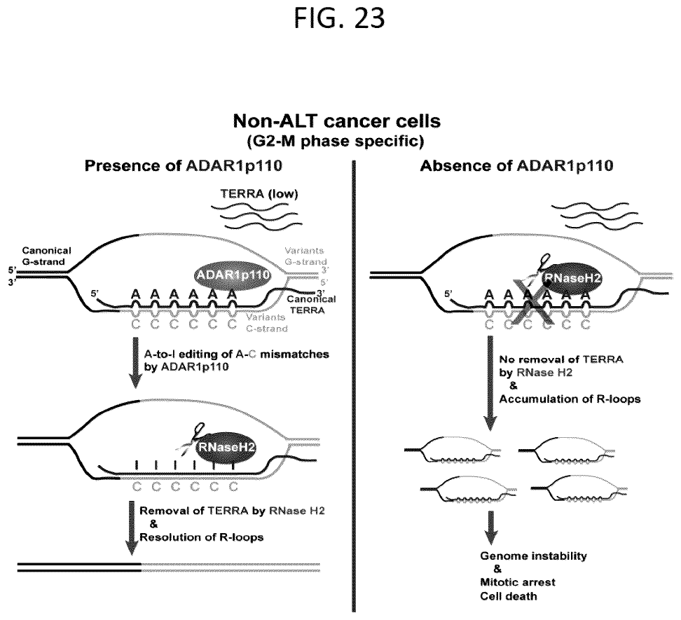

It is described herein that ADAR1p110 regulates R-loop formation and genome stability at telomeres in cancer cells carrying non-canonical variants of telomeric repeats (FIG. 23). ADAR1p110 edits the A-C mismatches within RNA:DNA hybrids formed between canonical and non-canonical variant repeats. Editing of A-C mismatches to I:C matched pairs facilitates resolution of telomeric R-loops by RNase H2. This ADAR1p110-dependent control of telomeric R-loops is required for continued proliferation of telomerase-reactivated cancer cells, revealing the pro-oncogenic nature of ADAR1p110 and identifying ADAR1 as a therapeutic target of telomerase positive cancers.

Provided herein are constructs and cell lines which include the same which are useful for screening compounds to identify ADAR1 inhibitors. The components described herein are useful, in alternative embodiments, in nucleic acid constructs, transformed cancer cells, stable cell lines, and screening methods.

In one aspect, a modified cancer cell is provided. The cancer cell comprises a nucleic acid that comprises a dual reporter system, said dual reporter system comprising i) a first reporter, ii) an adenosine-to-inosine (A-to-I) editing site within a stop codon of a short hairpin dsRNA, and iii) a second reporter, wherein the first reporter and second reporter are different.

The cancer cell is selected from those that use telomerase for chromosome maintenance, also referred to herein as a “non-ALT cancer”. Most cancers rely on telomerase to extend and maintain telomeres, but 10-15% of cancers use a homologous recombination-based pathway called alternative lengthening of telomeres (ALT). As used herein, the term “non-ALT cancer” refers to a cancer that relies on telomerase for linear chromosome maintenance and sustained proliferation of telomeres. It is estimated that at least 70-80% of cancer types are non-ALT cancers. Such cancers include, without limitation, melanoma, bladder cancer, Bladder carcinoma, Renal pelvic carcinoma, urothelial carcinoma, Hepatocellular carcinoma, Skin basal cell carcinoma, thyroid cancer (papillary and poorly differentiated carcinomas), myxoid liposarcoma, glioblastoma, medulloblastoma, Oligoastrocytoma, Oligodendroglioma, Breast cancer, colorectal cancer, medullary thyroid carcinoma, ovarian cancer, esophageal adenocarcinoma, acute myeloid leukemia, chronic lymphoid leukemia, pancreatic cancer, prostate cancer, testicular carcinoma, uterine cervix cancer, pleomorphic dermal sarcoma, myxoid liposarcoma, glioma, urothelial cell carcinoma, carcinoma of the skin, liver cancer, gastric cancer, pancreatic cancer, non-small-cell lung cancer and gastrointestinal stromal tumors, prostate cancer, multiple myeloma, recurrent or metastatic breast cancer, solid tumor malignancies, refractory chronic lymphoproliferative disease, non-small-cell lung cancer, HER2⁺ breast cancer, Myelofibrosis, lung, liver, prostate and pancreas, glioblastoma, adenocarcinoma, and acute myelogenous leukemia, See, Jafri et al, Roles of telomeres and telomerase in cancer, and advances in telomerase-targeted therapies, Genome Med. 2016; 8: 69. Published online 2016 Jun. 20, which is incorporated herein by reference.

The cell lines used in assays of the invention may be used to achieve transient expression of the dual reporter system, or may be stably transfected with constructs that express dual reporter system. Means to generate stably transformed cell lines are well known in the art and such means may be used here. The introduction, which may be generally referred to without limitation as “transformation”, may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. General aspects of mammalian cell host system transformations have been described in U.S. Pat. No. 4,399,216. For various techniques for transforming mammalian cells, see Keown et al., Meth. Enzym., 185:527-537 (1990) and Mansour et al., Nature 336:348-352 (1988). In certain embodiments, the cancer cell is selected from a non-ALT cell line. Certain non-ALT cell lines are known in the art. They include, without limitation, HeLa (ATCC) and HeLal.2.11 cervical carcinoma cells, HT1080 fibrosarcoma cells (ATCC), HEK 293T embryonic kidney cells expressing SV40 large T antigen (ATCC), and HCT116 and DKO human colon carcinoma cells (see, Arora R, et al. RNaseH1 regulates TERRA-telomeric DNA hybrids and telomere maintenance in ALT tumour cells. Nat Commun. 2014 Oct. 21; 5:5220. doi: 10.1038/ncomms6220. PMID: 25330849, which is incorporated herein by reference). In one embodiment, the non-ALT cell line is a HeLa cell line.

The cancer cell includes a nucleic acid that comprises a dual reporter system which includes a first reporter and a second reporter, which differ. The reporters can be any directly or indirectly detectable molecule. Typically, the reporter can be detected optically as a visible, e.g., fluorescent product or by its ability to generate a visible or otherwise detectable product. Suitable reporters include green fluorescent protein and its variants (“GFP”), beta-galactosidase, beta-glucuronidase, nano luciferase, and luciferase. Common luciferases useful herein include firefly luciferase, click beetle luciferase, renilla luciferase, renilla mutant luciferase, gaussian luciferase, Olophorus luciferase (OLuc) and nano luciferase (NanoLuc). See, e.g., England et al, NanoLuc: A Small Luciferase Is Brightening Up the Field of Bioluminescence. Bioconjug Chem. 2016 May 18; 27(5):1175-1187. doi: 10.1021/acs.bioconjchem.6b00112. Epub 2016 Apr. 19. PMID: 27045664, which is incorporated herein by reference. In one embodiment, the first reporter is firefly luciferase and the second reporter is nano luciferase. In another embodiment, the first reporter is nano luciferase and the second reporter is firefly luciferase.

Importantly, the cancer cell also provides a site for known ADAR1 A-to-I RNA editing, in a highly sensitive and quantitative manner. Adenosine-to-inosine (A-to-I) RNA editing is a co-/posttranscriptional modification of double-stranded RNA (dsRNA), which is catalyzed by the adenosine deaminases acting on RNA (ADAR) family of enzymes and is the most abundant form of RNA editing in higher eukaryotes. ADAR enzymes deaminate adenosine bases to inosine, which is recognized as guanosine by ribosomes and splicing machinery. As such, RNA editing can induce non-synonymous amino acid changes resulting in differential protein isoform expression and thus is considered a key mechanism of transcriptome and proteome diversification in metazoans. For example, ADAR editing of the Q/R site in the GluA2 mRNA modifies a glutamine codon with the consequence that arginine is incorporated since inosine is read as guanosine by the translational machinery. Fritzell et al described the development of a bioluminescent reporter system the highly sensitive and quantitative Nanoluciferase that is conditionally expressed upon reporter-transcript editing of GluA2. Stably introduced into cancer cell lines, the system reports on elevated endogenous ADAR1 editing activity induced by interferon as well as knockdown of ADAR1 and ADAR2 (Fritzell K, Sensitive ADAR editing reporter in cancer cells enables high-throughput screening of small molecule libraries. Nucleic Acids Res. 2019 Feb. 28; 47(4):e22. doi: 10.1093/nar/gky1228. PMID: 30590609; PMCID: PMC6393238, which is incorporated herein by reference).

Various ADAR1 mediated-A-to-I editing sites are known in the art. They include those shown in Table 1 below. In certain embodiments, the dual reporter system includes an adenosine-to-inosine (A-to-I) editing site within a stop codon of a short hairpin dsRNA which includes one of these sequences. In one embodiment, the dual reporter system includes an adenosine-to-inosine (A-to-I) editing site within a stop codon of a short hairpin dsRNA of GluR2. In one embodiment, the dual reporter system includes an adenosine-to-inosine (A-to-I) editing site within a stop codon of a short hairpin dsRNA of GluR3. In one embodiment, the dual reporter system includes an adenosine-to-inosine (A-to-I) editing site within a stop codon of a short hairpin dsRNA of GluR4. In one embodiment, the dual reporter system includes an adenosine-to-inosine (A-to-I) editing site within a stop codon of a short hairpin dsRNA of GluR5. In one embodiment, the dual reporter system includes an adenosine-to-inosine (A-to-I) editing site within a stop codon of a short hairpin dsRNA of GluR6.

TABLE 1

| | | | ADAR | | |
|---|---|---|---|---|---|
| Gene | Protein | Recoding | responsible | Function | Reference |
| GRIA2 | GluR2 subunit of | Q→R | ADAR2 | Change in | 81 |
| | AMPA glutamate | | | Ca²⁺ permeability | |
| | receptor | R→G | ADAR1, | Change in | 82 |
| | | | ADAR2 | receptor | |
| | | | | desensitization | |
| GRIA3 | GluR3 subunit of | R→G | ADAR1, | Change in | 82 |
| | AMPA glutamate | | ADAR2 | receptor | |
| | receptor | | | desensitization | |
| GRIA4 | GluR4 subunit of | R→G | ADAR1, | Change in | 82 |
| | AMPA glutamate | | ADAR2 | receptor | |
| | receptor | | | desensitization | |
| GRIK1 | GluR5 subunit of | Q→R | ADAR1, | Change in | 83 |
| | kainate glutamate | | ADAR2 | Ca²⁺ permeability | |
| | receptor | | | | |
| GRIK2 | GluR6 subunit of | Q→R | ADAR1, | Change in | 84, 85 |
| | kainate glutamate | | ADAR2 | Ca²⁺ permeability | |
| | receptor | I→V | ADAR1, | | |
| | | | ADAR2 | | |
| | | Y→C | ADAR2 | | |
| HTR2C | Serotonin | I→V, M | ADAR1 | Change in G | 86 |
| | receptor 2C | N→S, G, | ADAR1, | protein-coupling | |
| | | D | ADAR2 | functions | |
| | | I→V | ADAR2 | | |
| KCNA1 | Voltage-gated | I→V | ADAR2 | Change in | 87 |
| | K⁺ channel | | | channel | |
| | (Kv1.1) | | | inactivation | |
| GABRA3 | GABAA receptor, | I→M | ADAR1, | Kinetics of | 88 |
| | subunit α3 | | ADAR2 | activation and | |
| | | | | inactivation, | |
| | | | | receptor | |
| | | | | trafficking | |
| BLCAP | Bladder cancer- | Y→C | ADAR1, | Not determined | 89, 90 |
| | associated protein | | ADAR2 | | |
| | | Q→R | ADAR1, | | |
| | | | ADAR2 | | |
| | | K→R | ADAR1, | | |
| | | | ADAR2 | | |
| CYFIP2 | Cytoplasmic | K→E | ADAR2 | Not determined | 89 |
| | FMR1-interacting | | | | |
| | protein 2 | | | | |
| FLNA | Filamin-α | Q→R | ADAR1, | Not determined | 89 |
| | | | ADAR2 | | |
| FLNB | Filamin-β | M→V | ADAR1, | Liver cancer | 91 |
| | | | ADAR2 | progression | |
| COPA | Coatomer protein | I→V | ADAR2 | Suppression of | 91 |
| | complex subunit-α | | | liver cancer | |
| IGFBP7 | Insulin-like | K→R | — | Proteolytic | 89 |
| | growth factor- | R→G | | cleavage | |
| | binding protein 7 | | | sensitivity | |
| AR | Androgen | T→A | ADAR1, | Prostate cancer | 92 |
| | receptor | | ADAR2 | progression | |
| | | | | Inhibition of | |
| | | | | interaction with | |
| | | | | androgen ligands | |
| AZIN1 | Antizyme | S→G | ADAR1 | Liver cancer | 93 |
| | inhibitor 1 | | | progression | |
| | | | | Change in | |
| | | | | affinity for | |
| | | | | antizyme | |
| NEIL1 | DNA repair | K→R | ADAR1 | Change in | 94 |
| | enzyme NEI-like | | | efficiency or | |
| | protein 1 | | | specificity of | |
| | | | | damaged base | |
| | | | | removal | |
| GLI1 | Glioma- | R→G | ADAR1, | Increased | 95 |
| | associated | | ADAR2 | transcription | |
| | oncogene 1 | | | enhancement | |
| RHOQ | RAS homology | N→S | — | Colorectal cancer | 96 |
| | family member Q | | | metastasis | |
| | | | | Disruption of | |
| | | | | interaction with | |
| | | | | Rap-RapGAP | |

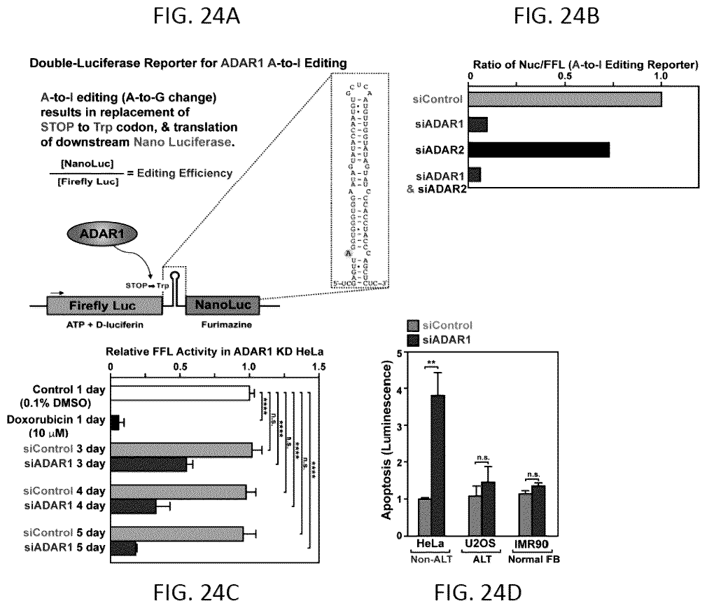

The A-to-I editing site is contained within a stop codon (UAG or UGA) at an adenosine base. In the absence of A-to-I editing, transcription is terminated at the stop codon. The dual reporter is designed such that upon completion of A-to-I editing, the stop codon is recoded to Trp, resulting in in-frame translation of the second reporter, which is downstream of the A-to-I editing site. In the cell line exemplified herein, the reporter utilizes the natural GluA2 editing substrate in which the R/G editing site was modified into a stop codon (UAG) that upon editing is recoded into a tryptophan codon (UGG). A first reporter gene upstream of the edited site monitors translation and a second reporter gene downstream is used to measure read-through after editing. Editing is measured as the ratio between luminescence from the second reporter and the first reporter (FIG. 24B).

In addition to the coding sequences for the reporters, the dual reporter system also includes regulatory sequences necessary for expression of the reporters. Such sequences include a promoter, and optionally other sequences such as a polyA, enhancer, etc.

The dual reporter system is designed such that the first reporter is upstream from the adenosine-to-inosine (A-to-I) editing site within a stop codon of a short hairpin dsRNA which is upstream from the second reporter. Each of these components is under control of a single promoter. When A-to-I editing does not occur (in the absence of ADAR1), the stop codon remains in place, and translation stops prior to initiation of translation of the second reporter. When A-to-I editing does occur (in the presence of ADAR1), the stop codon is replaced with a Trp codon, and translation of the second reporter occurs (FIG. 24A).

### Screening Method

Provided herein is a multi-step screening method for identifying ADAR1 inhibiting compounds. This method takes advantage of the fact that ADAR1 controls R loops only in non-ALT cancer cells, as further described herein. Thus, ADAR1 inhibitory compounds will selectively kill non-ALT cancer cells.

Thus, in one embodiment, a screening method is provided. The method includes one or more of the following individual steps, each in itself a method (referred to as steps). It should be understood that the following steps can be practiced in any order, with reference to first, second, third, etc., being for convenience.

The first step of the method includes providing a test compound to a non-ALT cancer cell (or population of cells) comprising a nucleic acid that comprises a dual reporter system, said dual reporter system comprising i) a first reporter, ii) an adenosine-to-inosine (A-to-I) editing site within a stop codon of a short hairpin dsRNA, and iii) a second reporter, wherein the first reporter and second reporter are different, and wherein adenosine to inosine editing alters the stop codon, resulting in in-frame translation of the second reporter, and measuring the level of the first reporter about three days after providing the test compound, wherein a reduction of the level of the first reporter of at least about 30% as compared to a control identifies an inhibitor of ADAR1. In another embodiment, a reduction of 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more identifies an inhibitor of ADAR1. In this step, a positive control is a strong inducer of apoptosis in the cell line used, as the measurement of the first reporter is used as a measure of cell viability or apoptosis. In one embodiment, the control compound is doxorubicin.

The level of the first reporter is assessed about 3 days after providing the test compound. In other embodiments, the level of the first reporter is assessed about 60 hours, 61 hours, 62 hours, 63 hours, 64 hours, 65 hours, 66 hours, 67 hours, 68 hours, 69 hours, 70 hours, 71 hours, 72 hours, 73 hours, 74 hours, 75 hours, 76 hours, 77 hours, 78 hours, 79 hours, 80 hours or more after providing the compound to the cells.

In alternative embodiments, the cells are assessed for apoptosis using a different method about 3 days after providing the test compound. In other embodiments, the cells are assessed for apoptosis 60 hours, 61 hours, 62 hours, 63 hours, 64 hours, 65 hours, 66 hours, 67 hours, 68 hours, 69 hours, 70 hours, 71 hours, 72 hours, 73 hours, 74 hours, 75 hours, 76 hours, 77 hours, 78 hours, 79 hours, 80 hours or more after providing the compound to the cells.

The levels of the first and second reporter are assessed using techniques that are known in the art and which vary according to the reporters utilized in the cell line and screening method. Where necessary, the required substrate is added to the cells. For example, when firefly luciferase is used as a reporter, D-luciferin is added to the cells, and bioluminescence is measured. Where NanoLuc is used, furimazine is added to the cells, and bioluminescence is measured. Instruments, reagents and techniques for measuring levels of visual reporters are well known in the art. For example, the Steadylite Plus FFL assay system, Nano Glo Dual Luciferase assay system, etc. Table 2 below provides common luciferase reporters and their substrates.

TABLE 2

| Luciferase | Substrate | Emission | Comments |
|---|---|---|---|
| Firefly (Photinus pyralis) Fluc Click beetle (Pyrophorus plagiophthalamus) green CBGr99 Click beetle red Cbred | D-luciferin 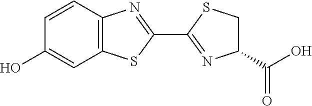 [SMILES: O=C(O)[C@H]1CSC(c2nc3ccc(O)cc3s2)=N1] | 612 nm 611 nm 544 nm | Requires ATP; Substrate has to penetrate cells; Optimized for exresssion in mammalian cells; Versions with increased thermostability availability |
| Sea pansy (Renilla reniformis) Rluc Renilla mutant Rluc 8.6- 535 Mesopelagic copepod (Gaussia princeps) Gluc | Coelenterazine 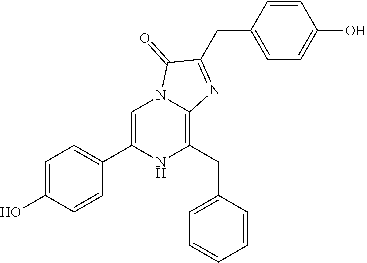 [SMILES: O=c1c(Cc2ccc(O)cc2)nc2c(Cc3ccccc3)[nH]c(-c3ccc(O)cc3)cn1-2] | 480 nm 535 nm 480 nm | Does not require ATP; substrate has to penetrate cells; commonly used for mammalian cells Secreted enzyme membrane localized versions available |
| NanoLuc (mutant Oplophorus gracilirostris) NLuc | Furimazine 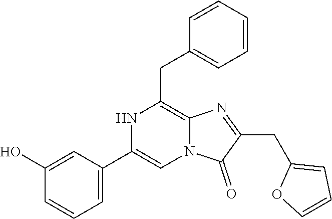 [SMILES: O=c1c(Cc2ccco2)nc2c(Cc3ccccc3)[nH]c(-c3cccc(O)c3)cn1-2] | | Small (19.1 kDa); enhanced stability; >150-fold increase in luminescence |
| Bacterial luciferase | Endogenous reduced flavin | 490 nm | Lux operon (luxCDABE) for |
| (Photorhabdus | mononucleotide | | Gram-negative (luxABCDE) |
| luminescens) lux | | | for |

The second step includes providing a test compound to a non-ALT cancer cell (or population of cells) comprising a nucleic acid that comprises a dual reporter system as described herein. In certain embodiments, the dual reporter system comprises i) a first reporter, 11) an adenosine-to-inosine (A-to-I) editing site within a stop codon of a short hairpin dsRNA, and iii) a second reporter, wherein the first reporter and second reporter are different, and wherein adenosine to inosine editing alters the stop codon to Trp, resulting in in-frame translation of the second reporter. The method further includes measuring the levels of expression of the first reporter and the second reporter wherein the ratio of the expression level of the second reporter to the ratio of the expression level of the first reporter demonstrates A-to-I editing efficiency.

The ratio between the level of the second reporter and the level of the first reporter (A-to-I editing efficiency) is calculated for each sample (e.g., a culture well) and results are normalized to a negative and positive control. The positive control may be an inhibitor of the second reporter known in the art, such as those for NanoLuciferase (e.g., Walker, J. R., Hall, M. P., Zimprich, C. A., Robers, M. B., Duellman, S. J., Machleidt, T., Rodriguez, J. and Zhou, W. (2017) Highly potent Cell-Permeable and impermeable NanoLuc luciferase inhibitors. ACS Chem. Biol., 12, 1028-1037, incorporated herein by reference).

The editing efficiency may be assessed about 1 day after providing the compound to the cells. In other embodiments, editing efficiency is assessed about 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours or more after providing the compound to the cells. In other embodiments, editing efficiency is assessed 1, 2, 3, or 4 days after providing the compound to the cells.

A reduction of at least about 60% of the reporter activity (e.g., Nluc/FFL=A-to-I editing activity) as compared to the control identifies an inhibitor of ADAR1. In one embodiment, a reduction of 60% or more identifies an inhibitor of ADAR1. In another embodiment, a reduction of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more identifies an inhibitor of ADAR1.

The third step of the method includes providing a test compound to a non-ALT cancer cell (or population of cells) comprising a nucleic acid that comprises a dual reporter system, said dual reporter system comprising a first reporter, an adenosine-to-inosine (A-to-I) editing site within a stop codon of a short hairpin dsRNA, and a second reporter, wherein the first reporter and second reporter are different, and wherein adenosine to inosine editing alters the stop codon, resulting in in-frame translation of the second reporter, and measuring the level of the first reporter about one day after providing the test compound, wherein a reduction of the level of the first reporter of less than about 40% (0% up to 40% decrease) as compared to a control identifies an inhibitor of ADAR1.

The level of the first reporter Is assessed about 1 day after providing the test compound. In other embodiments, the level of the first reporter is assessed about 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours or more after providing the compound to the cells.

In alternative embodiments, the cells are assessed for apoptosis using a different method about 1 day after providing the test compound. In other embodiments, the cells are assessed for apoptosis 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours or more after providing the compound to the cells.

In certain embodiments, cell viability is assessed with an assay that may include, but is not limited to, dye uptake assays (e.g., calcein AM assays), XTT cell viability assays, and dye exclusion assays (e.g., trypan blue, Eosin, or propidium dye exclusion assays). In particular embodiments, a viable cell has negative expression of one or more apoptotic markers, e.g., annexin V or active Caspase 3. In some embodiments, the viable cell is negative for the expression of one or more apoptosis marker that may include, but are not limited to, a caspase, e.g., caspase 2, caspase 3, caspase 6, caspase 7, caspase 8, caspase 9, and caspase 10, Bcl-2 family members, e.g., Bax, Bad, and Bid, Annexin V, and/or TUNEL staining.

In particular embodiments, the apoptotic marker may include any known marker associated with apoptosis, and may include expression of genes, proteins, or active forms of proteins, or the appearance of features associated with apoptosis, such as blebbing and/or nuclear breakdown. In certain embodiments, the apoptotic marker is a marker associated with apoptosis that may include, but is not limited to, pro-apoptotic factors known to initiate apoptosis, members of the death receptor pathway, activated members of the mitochondrial (intrinsic) pathway, Bcl-2 family members such as Bax, Bad, and Bid, Fas, FADD, presence of nuclear shrinkage (e.g., monitored by microscope), presence of chromosomal DNA fragmentation (e.g., presence of chromosomal DNA ladder), or markers associated with apoptosis assays, e.g., TUNEL staining, and Annexin V staining. In some embodiments, the marker of apoptosis is caspase expression, e.g., expression of the active forms of caspase-1, caspase-2, caspase-3, caspase-7, caspase-8, caspase-9, caspase-10 and/or caspase-13. In some embodiments, the apoptotic marker is Annexin V. In certain embodiments, the apoptotic marker is active caspase-3. Ward et al. Biomarkers of apoptosis. Br J Cancer. 2008 Sep. 16; 99(6): 841-846, which is incorporated herein by reference.

The test compound provided in the second/third step is the same test compound provided in the first step, such that the results generated for the first, second, and third steps are cumulative, allowing a stepwise reduction in the number of compounds in the screen.

The cells of the first step and the second step may be the same sample of cells. If different cells are used for the first and second steps, identical conditions for each population of cells are maintained.

In certain embodiments, the claimed method is directed to screening cells that are located in a multi-well vessel. The multi-well vessels of the claimed invention may contain up to and a number equaling 96 wells. In another embodiment, the multi-well vessel comprises greater than 96 wells. In another embodiment, the multi-well vessel comprises 384 wells. In yet another embodiment, the multi-well vessel comprises 1536 wells.

By “well” it is meant generally a bounded area within a container, which may be either discrete (e.g., to provide for an isolated sample) or in communication with one or more other bounded areas (e.g., to provide for fluid communication between one or more samples in a well). For example, cells grown on a substrate are normally contained within a well that may also contain culture medium for living cells. Substrates can comprise any suitable material, such as plastic, glass, and the like. Plastic is conventionally used for maintenance and/or growth of cells in vitro.

A “multi-well vessel”, as noted above, is an example of a substrate comprising more than one well in an array. Multi-well vessels useful in the invention can be of any of a variety of standard formats (e.g., plates having 2, 4, 6, 24, 96, 384, or 1536, etc., wells), but can also be in a non-standard format (e.g., plates having 3, 5, 7, etc., wells).

Test compounds employed in the screening methods of this invention include for example, without limitation, synthetic organic compounds, chemical compounds, naturally occurring products, polypeptides and peptides, nucleic acids, etc. Essentially any chemical compound can be used as a potential modulator or ligand in the assays of the invention. Most often compounds dissolved in aqueous or organic (especially dimethyl sulfoxide- or DMSO-based) solutions are used. The assays are designed to screen large chemical libraries by automating the assay steps. The compounds are provided from any convenient source to the cells. The assays are typically run in parallel (e.g., in microtiter formats on microtiter plates in robotic assays with different test compounds in different wells on the same plate). It will be appreciated that there are many suppliers of chemical compounds, including ChemDiv (San Diego, Calif.), Sigma-Aldrich (St. Louis, Mo.), Fluka Chemika-Biochemica-Analytika (Buchs Switzerland) and the like.

“Upregulate” and “upregulation”, as used herein, refer to an elevation in the level of expression of a product of one or more genes in a cell or the cells of a tissue or organ.

“Inhibit” or “downregulate”, as used herein refer to a reduction in the level of expression of a product of one or more genes in a cell or the cells of a tissue or organ.

By the general terms “blocker”, “inhibitor” or “antagonist” is meant an agent that inhibits, either partially or fully, the activity or production of a target molecule, e.g., as used herein, e.g., ADAR1. In particular, these terms refer to a composition or compound or agent capable of decreasing levels of gene expression, mRNA levels, protein levels or protein activity of the target molecule. Illustrative forms of antagonists include, for example, proteins, polypeptides, peptides (such as cyclic peptides), antibodies or antibody fragments, peptide mimetics, nucleic acid molecules, antisense molecules, ribozymes, aptamers, RNAi molecules, and small organic molecules. Illustrative non-limiting mechanisms of antagonist inhibition include repression of ligand synthesis and/or stability (e.g., using, antisense, ribozymes or RNAi compositions targeting the ligand gene/nucleic acid), blocking of binding of the ligand to its cognate receptor (e.g., using anti-ligand aptamers, antibodies or a soluble, decoy cognate receptor), repression of receptor synthesis and/or stability (e.g., using, antisense, ribozymes or RNAi compositions targeting the ligand receptor gene/nucleic acid), blocking of the binding of the receptor to its cognate receptor (e.g., using receptor antibodies) and blocking of the activation of the receptor by its cognate ligand (e.g., using receptor tyrosine kinase inhibitors). In addition, the blocker or inhibitor may directly or indirectly inhibit the target molecule.

In one embodiment, the high throughput screening methods involve providing a small organic molecule or peptide library containing a large number of potential ADAR1 modulators. Such “chemical libraries” are then screened in one or more assays, as described herein, to identify those library members (particular chemical species or subclasses) that display a desired characteristic activity. The compounds thus identified can serve as conventional “lead compounds” or can themselves be used as potential or actual products.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14:309-314 (1996) and PCTIUS96/10287), carbohydrate libraries (see, e.g., Liang et al., Science, 274:1520-1522 (1996) and U.S. Pat. No. 5,593,853), small organic molecule libraries (see, e.g., isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Candidate agents, compounds, drugs, and the like encompass numerous chemical classes, though typically they are organic molecules, preferably small organic compounds having a molecular weight of more than 100 and less than about 10,000 daltons, preferably, less than about 2000 to 5000 daltons. Candidate compounds may comprise functional groups necessary for structural interaction with proteins, particularly hydrogen bonding, and typically include at least an amine, carbonyl, hydroxyl or carboxyl group, preferably at least two of the functional chemical groups. The candidate compounds may comprise cyclical carbon or heterocyclic structures, and/or aromatic or polyaromatic structures substituted with one or more of the above functional groups. Candidate compounds are also found among biomolecules including peptides, saccharides, fatty acids, steroids, purines, pyrimidines, derivatives, structural analogs or combinations thereof.

A variety of other reagents may be included in the screening assay according to the present invention. Such reagents include, but are not limited to, salts, solvents, neutral proteins, e.g., albumin, detergents, etc., which may be used to facilitate optimal protein-protein binding and/or to reduce non-specific or background interactions. Examples of solvents include, but are not limited to, dimethyl sulfoxide (DMSO), ethanol and acetone, and are generally used at a concentration of less than or equal to 1% (v/v) of the total assay volume. In addition, reagents that otherwise improve the efficiency of the assay, such as protease inhibitors, anti-microbial agents, etc. may be used. Further, the mixture of components in the method may be added in any order that provides for the requisite binding.

### Pharmaceutical Compositions

In another aspect, a pharmaceutical composition comprising a pharmaceutically acceptable carrier and an ADAR1 inhibitor as described herein, is provided. In one embodiment, the ADAR1 inhibitor is [[amino-[3-chloro-4-[(4-chlorophenyl)methoxy]phenyl]methylidene]amino]5-nitrofuran-2-carboxylate (compound #1) or a prodrug, derivative, pharmaceutical salt, or analog thereof. In another embodiment, the ADAR1 inhibitor is 5,7-dimethyl-2-phenylpyrazolo[1,5-a]pyrimidine (compound #6) or a prodrug, derivative, pharmaceutical salt, or analog thereof. In another embodiment, the ADAR1 inhibitor is 3-chloro-N-(4-cyanophenyl)-1-benzothiophene-2-carboxamide (compound #13) or a prodrug, derivative, pharmaceutical salt, or analog thereof. In one embodiment, the ADAR1 inhibitor is Compound #13-analog 1 identified in Table 3. In one embodiment, the ADAR1 inhibitor is Compound #13-analog 2 identified in Table 3. In another embodiment, the ADAR1 inhibitor is 1-[(4-chlorophenyl)methylsulfanyl]-3-phenylpyrido[1,2-a]benzimidazole-4-carbonitrile (compound #23) or a prodrug, derivative, pharmaceutical salt, or analog thereof. In another embodiment, the ADAR1 inhibitor is N-(1-benzylpiperidin-4-yl)-6-phenylthieno[3,2-d]pyrimidin-4-amine (compound #24) or a prodrug, derivative, pharmaceutical salt, or analog thereof. In another embodiment, the ADAR1 inhibitor is 5-(4-chlorophenyl)-3-methylsulfanyl-1H-pyrazole-4-carbonitrile (compound #27) or a prodrug, derivative, pharmaceutical salt, or analog thereof. In another embodiment, the ADAR1 inhibitor is 4-[6-(3,5-dimethylpyrazol-1-yl)pyridazin-3-yl]thiomorpholine (compound #37) or a prodrug, derivative, pharmaceutical salt, or analog thereof. In another embodiment, the ADAR1 inhibitor is 3-(3,4,5-trimethoxyphenyl)-4,5-dihydro-1H-pyridazin-6-one (compound 940) or a prodrug, derivative, pharmaceutical salt, or analog thereof. In another embodiment, the ADAR1 inhibitor is 4-(furan-2-yl)-2,6-bis(methylsulfanyl)pyrimidine-5-carbonitrile (compound 952), or a prodrug, derivative, pharmaceutical salt, or analog thereof. The terms “analog”, “modification” and “derivative” refer to biologically active derivatives of the reference molecule that retain desired activity as described herein. Preferably, the analog, modification or derivative has at least the same desired activity as the native molecule, although not necessarily at the same level. The terms also encompass purposeful mutations that are made to the reference molecule.

TABLE 3
Identified compounds

| Compound | | |
|---|---|---|
| number | IUPAC | Structure |
| 1 | [[amino-[3-chloro-4-[(4- chlorophenyl)methoxy]phenyl]methylidene]a- mino]5-nitrofuran-2-carboxylate | 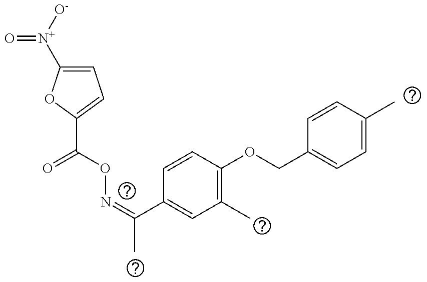 [SMILES: C/C(=N/OC(=O)c1ccc([N+](=O)[O-])o1)c1ccc(OCc2ccc(C)cc2)c(C)c1] |
| 6 | 5,7-dimethyl-2-phenylpyrazolo[1,5-a]pyrimidine | 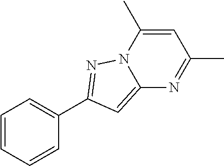 [SMILES: Cc1cc(C)n2nc(-c3ccccc3)cc2n1] |
| 13 | 3-chloro-N-(4-cyanophenyl)-1- benzothiophene-2-carboxamide | 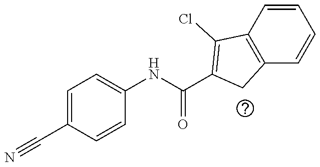 [SMILES: N#Cc1ccc(NC(=O)C2=C(Cl)c3ccccc3C2)cc1] |
| 13- analog 1 | | 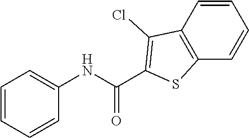 [SMILES: O=C(Nc1ccccc1)c1sc2ccccc2c1Cl] |
| 13- analog 2 | | 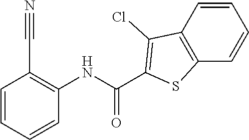 [SMILES: N#Cc1ccccc1NC(=O)c1sc2ccccc2c1Cl] |
| 23 | 1-[(4-chlorophenyl)methylsulfanyl]-3- phenylpyrido[1,2-a]benzimidazole-4-carbonitrile | 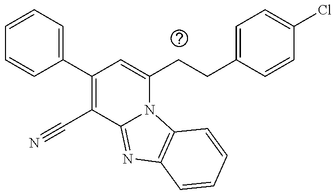 [SMILES: N#Cc1c(-c2ccccc2)cc(CCc2ccc(Cl)cc2)n2c1nc1ccccc12] |
| 24 | N-(1-benzylpiperidin-4-yl)-6- phenylthieno[3,2-d]pyrimidin-4-amine | 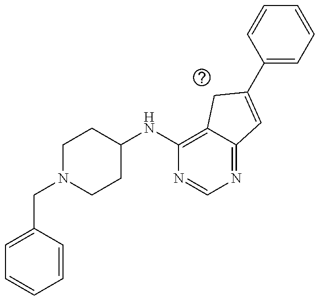 [SMILES: C1=C(c2ccccc2)Cc2c1ncnc2NC1CCN(Cc2ccccc2)CC1] |
| 27 | 5-(4-chlorophenyl)-3-methylsulfanyl-1H- pyrazole-4-carbonitrile | 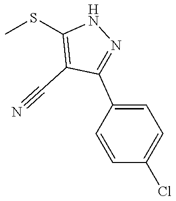 [SMILES: CSc1[nH]nc(-c2ccc(Cl)cc2)c1C#N] |
| 37 | 4-[6-(3,5-dimethylpyrazol-1-yl)pyridazin-3- yl]thiomorpholine | 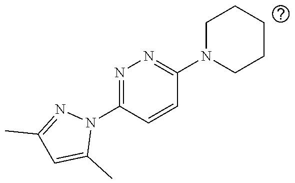 [SMILES: Cc1cc(C)n(-c2ccc(N3CCCCC3)nn2)n1] |
| 40 | 3-(3,4,5-trimethoxypehnyl)-4,5-dihydro-1H- pyridazin-6-one | 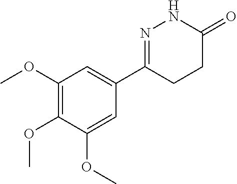 [SMILES: COc1cc(C2=NNC(=O)CC2)cc(OC)c1OC] |
| 52 | 4-(furan-2-yl)-2,6- bis(methylsulfanyl)pyimidine-5-carbonitrile | 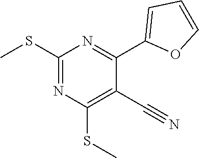 [SMILES: CSc1nc(SC)c(C#N)c(-c2ccco2)n1] |

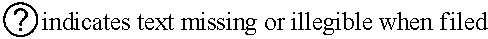

In another embodiment, a combination product is provided that includes a checkpoint inhibitor in addition to the ADAR1 inhibitor. Immune checkpoints represent significant barriers to activation of functional cellular immunity in cancer, and antagonistic antibodies specific for inhibitory ligands on T cells including CTLA4 and programmed death-1 (PD-1) are examples of targeted agents being evaluated in the clinics. In one embodiment, the subject has previously received checkpoint therapy, prior to receiving the ADAR1 inhibitor. The subject may, in some embodiments, receive the same or different checkpoint therapy after administration of the ADAR1 inhibitor.

Immune checkpoint molecules that may be targeted for blocking or inhibition include, but are not limited to, CTLA-4, 4-1BB (CD137), 4-1BBL (CD137L), PDL1, PDL2, PD1, CD134, B7-H3, B7-H4, BTLA, HVEM, TIM3, GAL9, LAG3, TIM3, B7H3, B7H4, VISTA, KIR, 2B4, CD160 (also referred to as BY55) and CGEN-15049. In one embodiment, the checkpoint inhibitor is a PD-1 inhibitor. In another embodiment, the checkpoint inhibitor is a PD-L1 inhibitor.

Immune checkpoint inhibitors include antibodies, or antigen binding fragments thereof, or other binding proteins, that bind to and block or inhibit the activity of one or more of CTLA-4, PDL1, PDL2, PD1, CD134, B7-H3, B7-H4, BTLA, HVEM, TIM3, GAL9, LAG3, TIM3, B7H3, B7H4, VISTA, KIR, 2B4, CD160 and CGEN-15049.

Suitable immune checkpoint inhibitors include those that block PD-1, such as pembrolizumab, nivolumab, AGEN 2034, BGB-A317, BI-754091, CBT-501 (genolimzumab), MEDIO680, MGA012, PDR001, PF-06801591, REGN2810 (SAR439684), and TSR-042. MK-3475 (PD-1 blocker) Nivolumab, and CT-011.

Immune checkpoint inhibitors also include those that block PD-L1, such as durvalumab, atezolizumab, avelumab, and CX-072. Other suitable inhibitors include Anti-B7-H1 (MEDI4736), AMP224, BMS-936559, MPLDL3280A, and MSB0010718C.

Suitable immune checkpoint inhibitors include those that block CTLA-4, such as AGEN 1884, ipilimumab, and tremelimumab.

In some embodiments, the immune checkpoint inhibitor is an anti-PD-1 antibody, an anti-PD-L1 antibody, an anti-CTLA-4 antibody, an anti-CD28 antibody, an anti-TIGIT antibody, an anti-LAGS antibody, an anti-TIM3 antibody, an anti-GITR antibody, an anti-4-1BB antibody, or an anti-OX-40 antibody. In some embodiments, the additional therapeutic agent is an anti-TIGIT antibody. In some embodiments, the additional therapeutic agent is an anti-LAG-3 antibody selected from the group consisting of: BMS-986016 and LAG525. In some embodiments, the additional therapeutic agent is an anti-OX-40 antibody selected from: MEDI6469, MEDI0562, and MOXR0916. In some embodiments, the additional therapeutic agent is the anti-4-1BB antibody PF-05082566.

The present disclosure provides compositions and methods that include blockade of immune checkpoints. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAGS), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example, it is known that lambrolizumab is also known under the alternative and equivalent names MK-3475 and pembrolizumab.

In addition, more than one immune checkpoint inhibitor (e.g., anti-PD-1 antibody and anti-CTLA-4 antibody) may be used in combination with the ADAR1 inhibitor.

In another embodiment, a combination product is provided which includes an ADAR1 inhibitor in combination with a chemotherapeutic agent, optionally in combination with a checkpoint inhibitor.

Chemotherapeutic agents (e.g., anti-cancer agents) are well known in the art and include, but are not limited to, anthracenediones (anthraquinones) such as anthracyclines (e.g., daunorubicin (daunomycin; rubidomycin), doxorubicin, epirubicin, idarubicin, and valrubicin), mitoxantrone, and pixantrone; platinum-based agents (e.g., cisplatin, carboplatin, oxaliplatin, satraplatin, picoplatin, nedaplatin, triplatin, and lipoplatin); tamoxifen and metabolites thereof such as 4-hydroxytamoxifen (afimoxifene) and N-desmethyl-4-hydroxytamoxifen (endoxifen); taxanes such as paclitaxel (taxol) and docetaxel; alkylating agents (e.g., nitrogen mustards such as mechlorethamine (HN2), cyclophosphamide, ifosfamide, melphalan (L-sarcolysin), and chlorambucil); ethylenimines and methylmelamines (e.g., hexamethylmelamine, thiotepa, alkyl sulphonates such as busulfan, nitrosoureas such as carmustine (BCNU), lomustine (CCNLJ), semustine (methyl-CC-U), and streptozoein (streptozotocin), and triazenes such as decarbazine (DTIC; dimethyltriazenoimidazolecarboxamide)); antimetabolites (e.g., folic acid analogues such as methotrexate (amethopterin), pyrimidine analogues such as fluorouracil (5-fluorouracil; 5-FU), floxuridine (fluorodeoxyuridine; fUdR), and cytarabine (cytosine arabinoside), and purine analogues and related inhibitors such as mercaptopurine (6-mercaptopurine; 6-MP), thioguanine (6-thioguanine; 6-TG), and pentostatin (“-deoxycofonnycin)); natural products (e.g., vinca alkaloids such as vinblastine (VLB) and vincristine, epipodophyllotoxins such as etoposide and teniposide, and antibiotics such as dactinomycin (actinomycin D), bleomycin, plicamycin (mithramycin), and mitomycin (mitomycin Q); enzymes such as L-asparaginase; biological response modifiers such as interferon alpha); substituted ureas such as hydroxyurea; methyl hydrazine derivatives such as procarbazine (N-methylhydrazine; MIH); adrenocortical suppressants such as mitotane (o,”-DDD) and aminoglutethimide; analogs thereof derivatives thereof and combinations thereof.

### Methods of Treatment

In one embodiment, the method includes inhibiting or reducing ADAR1 in a subject in need thereof. In one embodiment, the method includes administering an effective amount of an inhibitor of ADAR1. In one embodiment, the ADAR1 inhibitor is [[amino-[3-chloro-4-[(4-chlorophenyl)methoxy]phenyl]methylidene]amino]5-nitrofuran-2-carboxylate (compound #1) or a prodrug, derivative, pharmaceutical salt, or analog thereof. In another embodiment, the ADAR1 inhibitor is 5,7-dimethyl-2-phenylpyrazolo[1,5-a]pyrimidine (compound #6) or a prodrug, derivative, pharmaceutical salt, or analog thereof. In another embodiment, the ADAR1 inhibitor is 3-chloro-N-(4-cyanophenyl)-1-benzothiophene-2-carboxamide (compound #13) or a prodrug, derivative, pharmaceutical salt, or analog thereof. In another embodiment, the ADAR1 inhibitor is 1-[(4-chlorophenyl)methylsulfanyl]-3-phenylpyrido[1,2-a]benzimidazole-4-carbonitrile (compound #23) or a prodrug, derivative, pharmaceutical salt, or analog thereof. In another embodiment, the ADAR1 inhibitor is N-(1-benzylpiperidin-4-yl)-6-phenylthieno[3,2-d]pyrimidin-4-amine (compound #24) or a prodrug, derivative, pharmaceutical salt, or analog thereof. In another embodiment, the ADAR1 inhibitor is 5-(4-chlorophenyl)-3-methylsulfanyl-1H-pyrazole-4-carbonitrile (compound #27) or a prodrug, derivative, pharmaceutical salt, or analog thereof. In another embodiment, the ADAR1 inhibitor is 4-[6-(3,5-dimethylpyrazol-1-yl)pyridazin-3-yl]thiomorpholine (compound #37) or a prodrug, derivative, pharmaceutical salt, or analog thereof. In another embodiment, the ADAR1 inhibitor is 3-(3,4,5-trimethoxyphenyl)-4,5-dihydro-1H-pyridazin-6-one (compound #40) or a prodrug, derivative, pharmaceutical salt, or analog thereof. In another embodiment, the ADAR1 inhibitor is 4-(furan-2-yl)-2,6-bis(methylsulfanyl)pyrimidine-5-carbonitrile (compound #52), or a prodrug, derivative, pharmaceutical salt, or analog thereof.

In one embodiment, the effective amount of the ADAR1 inhibitor is an amount ranging from about 0.01 mg/ml to about 10 mg/ml, including all amounts therebetween and end points. In one embodiment, the effective amount of the ADAR1 inhibitor is about 0.1 mg/ml to about 5 mg/ml, including all amounts therebetween and end points. In another embodiment, the effective amount of the ADAR1 inhibitor is about 0.3 mg/ml to about 1.0 mg/ml, including all amounts therebetween and end points. In another embodiment, the effective amount of the ADAR1 inhibitor is about 0.3 mg/ml. In another embodiment, the effective amount of the ADAR1 inhibitor is about 0.4 mg/ml. In another embodiment, the effective amount of the ADAR1 inhibitor is about 0.5 mg/ml. In another embodiment, the effective amount of the ADAR1 inhibitor is about 0.6 mg/ml. In another embodiment, the effective amount of the ADAR1 inhibitor is about 0.7 mg/ml. In another embodiment, the effective amount of the ADAR1 inhibitor is about 0.8 mg/ml. In another embodiment, the effective amount of the ADAR1 inhibitor is about 0.9 mg/ml. In another embodiment, the effective amount of the ADAR1 inhibitor is about 1.0 mg/ml.

In one embodiment, the effective amount of the ADAR1 inhibitor is an amount ranging from about 1 μM to about 2 mM, including all amounts therebetween and end points. In one embodiment, the effective amount of the ADAR1 inhibitor is about 10 μM to about 100 μM, including all amounts therebetween and end points. In another embodiment, the effective amount of the ADAR1 inhibitor is about 5 μM. In another embodiment, the effective amount of the ADAR1 inhibitor is about 10 μM. In another embodiment, the effective amount of the ADAR1 inhibitor is about 20 μM. In another embodiment, the effective amount of the ADAR1 inhibitor is about 50 μM. In another embodiment, the effective amount of the ADAR1 inhibitor is about 100 μM. In another embodiment, the effective amount of the ADAR1 inhibitor is about 200 μM. In another embodiment, the effective amount of the ADAR1 inhibitor is about 300 μM. In another embodiment, the effective amount of the ADAR1 inhibitor is about 400 μM. In another embodiment, the effective amount of the ADAR1 inhibitor is about 500 μM. In another embodiment, the effective amount of the ADAR1 inhibitor is about 600 μM. In another embodiment, the effective amount of the ADAR1 inhibitor is about 700 μM. In another embodiment, the effective amount of the ADAR1 inhibitor is about 800 μM. In another embodiment, the effective amount of the ADAR1 inhibitor is about 900 μM. In another embodiment, the effective amount of the ADAR1 inhibitor is about 1 mM. In another embodiment, the effective amount of the ADAR1 inhibitor is about 1.25 mM. In another embodiment, the effective amount of the ADAR1 inhibitor is about 1.5 mM. In another embodiment, the effective amount of the ADAR1 inhibitor is about 1.75 mM. In another embodiment, the effective amount of the ADAR1 inhibitor is about 2 mM.

In certain embodiments, the subject is also treated with an effective amount of the checkpoint inhibitor. The checkpoint inhibitor can be any of those known in the art, or described herein. It should be understood that the “effective amount” for the checkpoint inhibitor may vary depending upon the agent(s) selected for use in the method, and may be determined by the person of skill in the art. In one embodiment an effective amount for the checkpoint inhibitor includes without limitation about 1p g to about 25 mg. In one embodiment, the range of effective amount is 0.001 to 0.01 mg. In another embodiment, the range of effective amount is 0.001 to 0.1 mg. In another embodiment, the range of effective amount is 0.001 to 1 mg. In another embodiment, the range of effective amount is 0.001 to 10 mg. In another embodiment, the range of effective amount is 0.001 to 20 mg. In another embodiment, the range of effective amount is 0.01 to 25 mg. In another embodiment, the range of effective amount is 0.01 to 0.1 mg. In another embodiment, the range of effective amount is 0.01 to 1 mg. In another embodiment, the range of effective amount is 0.01 to 10 mg. In another embodiment, the range of effective amount is 0.01 to 20 mg. In another embodiment, the range of effective amount is 0.1 to 25 mg. In another embodiment, the range of effective amount is 0.1 to 1 mg. In another embodiment, the range of effective amount is 0.1 to 10 mg. In another embodiment, the range of effective amount is 0.1 to 20 mg. In another embodiment, the range of effective amount is 1 to 25 mg. In another embodiment, the range of effective amount is 1 to 5 mg. In another embodiment, the range of effective amount is 1 to 10 mg. In another embodiment, the range of effective amount is 1 to 20 mg. Still other doses falling within these ranges are expected to be useful. The effective amount of the checkpoint inhibitor may be individually chosen based on the agent selected and other factors, e.g., size of the patient, type of cancer, etc.

In one embodiment, the ADAR1 inhibitor and checkpoint inhibitor are administered approximately simultaneously. In another embodiment, the ADAR1 inhibitor are administered prior to checkpoint inhibitor. In another embodiment, the ADAR1 inhibitor are administered subsequent to the checkpoint inhibitor.

In another embodiment, the method includes administering a chemotherapeutic agent to the subject in addition to the ADAR1 inhibitor, and optionally with a checkpoint inhibitor. Effective dosages for individual chemotherapeutic agents can be determined by the person of skill in the art.

In certain embodiments, the cancer being treated is a non-ALT cancer. In certain embodiments, the cancer treated includes, but is not limited to, a solid tumor, a hematological cancer (e.g., leukemia, lymphoma, myeloma, e.g., multiple myeloma), and a metastatic lesion. In one embodiment, the cancer is a solid tumor. Non-limiting examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma), and pancreas, as well as adenocarcinomas which include malignancies such as colon cancers, rectal cancer, renal-cell carcinoma, liver cancer, non-small cell lung cancer, cancer of the small intestine, cancer of the esophagus. The cancer may be at an early, intermediate, late stage or metastatic cancer.

The chemotherapeutic agents, ADAR1 inhibitors and checkpoint inhibitors may be administered using any suitable route of administration. For example, compositions may be administered via intravenous, parenteral, subcutaneous, intramuscular, intracranial, intraorbital, ophthalmic, intraventricular, intracapsular, intraspinal, intracisternal, intraperitoneal, intranasal, or aerosol administration. The route of administration for each composition (e.g., ADAR1 inhibitors, chemotherapeutic agents, checkpoint inhibitors) may be determined individually and may be the same or different.

A “subject” is a mammal, e.g., a human, mouse, rat, guinea pig, dog, cat, horse, cow, pig, or non-human primate, such as a monkey, chimpanzee, baboon or gorilla. The term “patient” may be used interchangeably with the term subject. In one embodiment, the subject is a human. The subject may be of any age, as determined by the health care provider. In certain embodiments described herein, the patient is a subject who has previously been diagnosed with cancer. The subject may have been treated for cancer previously, or is currently being treated for cancer. In one embodiment, the subject has a non-ALT cancer, e.g., a telomerase-activated cancer.

As used herein, the term “a therapeutically effective amount” refers an amount sufficient to achieve the intended purpose. An effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. The effective amount in each individual case may be determined by a skilled artisan according to established methods in the art.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remingto's Pharmaceutical Sciences, 1^{8t}h Ed., Gennaro, ed. (Mack Publishing Co., 1990). The formulation should suit the mode of administration.

In yet another embodiment, the methods described herein include treatment in combination with another cancer treatment or therapeutic agent to reduce or inhibit reversal of cancer cell dormancy, including known chemotherapeutic agents. The reduction or inhibition of cancer cell proliferation can be measured relative to the incidence observed in the absence of the treatment. The tumor inhibition can be quantified using any convenient method of measurement. Tumor inhibition can be reduced by about 10%, 20%, 30%, 40%, 50%, 60%, 70%, 80%, 90%, 95% or greater.

It is to be noted that the term “a” or “an” refers to one or more. As such, the terms “a” (or “an”), “one or more,” and “at least one” are used interchangeably herein.

While various embodiments in the specification are presented using “comprising” language, under other circumstances, a related embodiment is also intended to be interpreted and described using “consisting of” or “consisting essentially of” language. The words “comprise”, “comprises”, and “comprising” are to be interpreted inclusively rather than exclusively. The words “consist”, “consisting”, and its variants, are to be interpreted exclusively, rather than inclusively.

As used herein, the term “about” means a variability of 10% from the reference given, unless otherwise specified.

Unless defined otherwise in this specification, technical and scientific terms used herein have the same meaning as commonly understood by one of ordinary skill in the art and by reference to published texts, which provide one skilled in the art with a general guide to many of the terms used in the present application.

The following examples are illustrative only and are not intended to limit the present invention.

### EXAMPLES

### Example 1: Materials and Methods

### Cell Lines and Cell Culture Reagents

HeLa human ovarian carcinoma (ATCC CCL-2), HEK293T human embryonic kidney (ATCC CRL-11268), HCT116 human colon carcinoma (ATCC CCL-247), HT1080 human fibrosarcoma (ATCC CCL-121), U2OS human osteosarcoma (ATCC HTB-96), W138-VA13 human virus-transformed fibroblasts (ATCC CCL-75.1), Saos2 human osteosarcoma (ATCC HTB-85), WI38 lung fibroblast (ATCC CCL-75), and IMR90 lung fibroblast cells (ATCC CCL-186) were used in this study. Adar1^{-/-} MEF cells and isogenic control cells were established from Adar1^{-/-} mice¹⁸. Adar2^{-/-} MEF cells and isogenic control cells were established from Adar2^{-/-} mice⁴¹. HEK293T cells expressing FLAG-ADAR1p110-WT, FLAG-ADAR1p110-EAA, FLAG-ADAR2-WT, or FLAG-ADAR2-EAA were established by co-transfection of various p3XFLAG-CMV-10 plasmids (Sigma) with a puromycin resistance plasmid pPUR (Clontech)⁶⁹. These cell lines were free of Mycoplasma contamination.

HeLa, HEK293T, HCT116, HT1080, Saos2, IMR90, Adar1^{-/-} MEF, Adar2^{-/-} MEF cells, and isogenic control MEF cells were cultured in Dulbecco's modified Eagle's medium (DMEM) supplemented with 10% fetal bovine serum (Gemini), penicillin (100 U/ml), and streptomycin (100 μg/ml) at 37° C. in a humidified atmosphere with 5% CO₂. U2OS, W138-VA13, and W138 cells were cultured in DMEM/F12 supplemented with 10% fetal bovine serum, penicillin (100 U/ml), and streptomycin (100 μg/ml).

HEK293T cells expressing FLAG-ADAR1p110-WT were treated with thymidine and nocodazole to synchronize in the M phase. The cells were cultured in T175 flask and treated with 2.5 mM of thymidine (Sigma) for 24 h. To release from the thymidine block, the cells were washed twice with phosphate-buffered saline (PBS) and culture medium. After incubation with a fresh medium for 3 h, the cells were treated with 0.1 g/ml of nocodazole (Sigma) for 12 h.

### Plasmid Construction

An NheI restriction site was added to the multi-cloning site (MCS) of CSII-EF-MCS-IRES-puromycin-resistant gene (puro) by inserting the new MCS site oligonucleotide into NotI-BamHI-digested CSII-EF-MCS-IRES-puro vector^{70,71}. CSII-EF-FLAG-ADAR1p110-WT-IRES-puro, CSII-EF-FLAG-ADAR1p110-E912A-IRES-puro, or CSII-EF-FLAG-ADAR1p150-WT-IRES-puro used for protein overexpression in human cells was prepared by PCR cloning using p3XFLAG-CMV10-ADAR1p110-WT, p3XFLAG-CMV10-ADAR1p110-E912A, or p3XFLAG-CMV10-ADAR1p150-WT, respectively⁶⁹. The FLAG-ADAR1p110 PCR products were amplified using primers CSII-FLAG-p110-F and CSII-FLAG-p110-R. The PCR products were digested with NotI and BamHI and then inserted into CSII-EF-MCS-IRES-puro. The FLAG-ADAR1p150 PCR products were amplified using primers CSII-p150-F and CSII-p150-R. The PCR products were digested with NotI and NheI and then inserted into CSII-EF-MCS-IRES-puro. CSII-EF-FLAG-rNaseH2A was prepared by PCR cloning using pcDNA3.1-rNaseH2A⁷² The FLAG-rNaseH2A PCR products were amplified using primers CSII-FLAG-rNaseH2A-F and CSII-rNaseH2A-R. The PCR products were digested with NotI and BamHI and then inserted into CSII-EF-MCS-IRES-puro. CSII lentivirus vector was a kind gift from Hiroyuki Miyoshi and Toru Nakano.

pET28-FLAG-rNaseH2A used for recombinant protein purification was prepared by PCR cloning using a pET28-His-rNaseH2A plasmid. The PCR products were amplified using primers pET28-FLAG-rNaseH2A-F and FLAG-rNaseH2A-R, digested with XbaI and XhoI, and inserted into pET28 vector⁷³. pET28-His-RNASEH2A and pET15-His-rNaseH2B/2C were kind gifts from Marcin Nowotny.

### Gene Knockdown

Gene knockdown experiments were done by RNA interference methods using Lipofectamine RNAiMax (Life Technologies) or HiperFect at a final short interfering RNA (siRNA) concentration of 1, 2, or 5 nM.

### Lentivirus Infection

HEK293FT cells (5-6×10⁶) incubated in a 10 cm dish for a confluency of 80% were transfected with the following three plasmids using Lipofectamine 3000: 17 μg of CSII-EF-FLAG-ADAR1 or CSII-EF-FLAG-rNaseH2A plasmid, 10 g of pCAG-HIVgp (GAG-POL DNA), and 10 g of the vesicular stomatitis virus G (VSV-G) envelope plasmid pCMV-VSV-G. After 48 h incubation, the cell culture supernatant was filtered using a 0.45 m filter and concentrated by Lenti-X concentrator (Clontech). The lentiviral pellet was resuspended in 2 ml of fresh culture medium containing 8 μg/ml of polybrene (Sigma) and added to 1×10⁵ cells cultured in a 6-well plate. Infected cells were then incubated with puromycin (1 μg/ml) for 48 h post infection for antibiotic selection. The extent of infection of CSII-EF-FLAG-ADAR1 or CSII-EF-FLAG-rNaseH2A was evaluated by western blotting analysis and immunostaining with anti-FLAG M2 antibody. ADAR1 rescue experiments required exogenous FLAG-ADAR1 expression in every cell and were carried out using early passage cells (<passage 6).

### Immunofluorescence Staining

Transfection of siRNAs (siADAR1-1) into HeLa cells at 1 nM concentration was carried out as described above. After incubation for 24 h, the culture medium was replaced with a fresh medium containing CellLight Tubulin-GFP and BacMam 2.0 (Thermo Fisher Scientific). Nuclei were stained with SiR-DNA reagent (Cytoskeleton) at 0.25 μM for 6 h. Cells were cultured on Ibidi pDish 3.5 cm. After 72 h, cells were fixed with 4% paraformaldehyde and soaked in Dulbecco's PBS. Microscopic images were obtained by using a Leica TCS SP5 DMI6000 CS Confocal Microscope and LAS X software (Leica), equipped with ultraviolet 405 diode, Argon, DPS3561, and HeNe594 lasers. Fluorescent images were captured with a 40× lens with a 512×512 frame. For multicolor experiments, the following wavelength settings were used: Tubulin-GFP (Ex 488 nm/Em 498-630 nm) and SiR-DNA reagent (Ex 647 nm/Em 657-800 nm). Nuclear morphological analysis was performed using 4′,6-diamidino-2-phenylindole (DAPI)-stained HeLa cells.

### Immunoblot Analysis

Cell lysates were prepared in Laemmli buffer containing benzonase nuclease (Sigma), complete EDTA-free proteinase inhibitor cocktail (Roche), and PhosStop phosphatase inhibitor cocktail (Roche) and size-fractionated by 4-20% sodium dodecyl sulfate-polyacrylamide gel electrophoresis. Proteins were blotted to Immobilon-P nylon membrane (Millipore). Membranes were blocked with 10% Blocker BSA (bovine serum albumin) buffer (Thermo Fisher Scientific) and incubated with the primary antibodies overnight at 4° C. After incubation with each appropriate secondary antibody, bands were detected with ECL (GE Healthcare) using X-ray films. Antibodies were diluted in 10% Blocker BSA buffer (Thermo Fisher Scientific).

Dot blot analysis of genomic DNA Cells were treated with siRNA for 72 h in a 10 cm dish, detached from the dish surface with TrypLE Express Enzyme, and harvested by centrifugation. After PBS wash, genomic DNA was purified using QIAGEN Blood & Cell Culture DNA Midi Kit. Briefly, the cell pellet was resuspended in buffer C1. After repeated buffer C1 wash and removal of the cell debris, the nuclear pellet was resuspended in buffer G2 (without rNase A) and treated with 2 mg of proteinase K at 50° C. for 60 min. The nuclear fraction was applied to a buffer QBT-treated QIAGEN Genomic-tip and washed twice with buffer QC. Genomic DNA was eluted with buffer QF and precipitated with 2-propanol. The DNA pellet was washed twice with 80% ethanol and air-dried. Genomic DNA was dissolved in TE buffer (10 mM Tris-HCl pH 8.0, 1 mM EDTA) and incubated overnight at 4° C.

Genomic DNA was diluted in 100 μl of 6× saline sodium citrate (SSC) and spotted onto a Hybond N⁺ (GE Healthcare) using a Bio-Dot Apparatus (#1706545, Bio-Rad). The membrane was cross-linked with ultraviolet (UV) (0.24 J) and blocked with 5% non-fat dry milk (LabScientific) in PBS with 0.1% Tween-20 (PBST) for 1 h and then with SuperBlock buffer (Thermo Fisher Scientific) for 1 h at room temperature. The membrane was incubated with S9.6 antibody (Sigma) at 0.1 g/ml in SuperBlock buffer overnight at 4° C. After washing three times with PBST, the membrane was incubated with horseradish peroxidase-conjugated donkey anti-mouse IgG secondary antibody (Jackson Immuno Research) (0.04 g/ml) at 0.1 g/ml in SuperBlock buffer for 1 h at room temperature. After washing four times with PBST, dot signals were detected with ECL (GE Healthcare) using X-ray films. For the treatment with rNase H, 1 μg of genomic DNA was preincubated with 2 U of E. coli-rNase H (NEB) for 2 h at 37° C. DNA:DNA, RNA:RNA, or RNA:DNA oligo duplex controls were annealed in buffer containing 10 mM Tris-HCl (pH 7.6) and 50 mM NaCl at 80° C. for 5 min, followed by slow cooling to room temperature.

### DNA:RNA Hybrid Immunoprecipitation

Fifty micrograms of genomic DNA prepared as described above was diluted in 250 μl of sonication buffer (10 mM Tris-HCl pH 8.5, 300 mM NaCl) and sonicated using Bioruptor (Diagenode) (20 cycles at high power, 30 s ON/60 s OFF) or Sonicator W-220 (Heat Systems Ultrasonics) (20 cycles at Lv3.5, 10 s ON/40 s OFF). During sonication, samples were kept cold very carefully. Sonicated genomic DNA was mixed with 0.02 pmol spike RNA:DNA oligonucleotide duplexes. A fraction of the genomic DNA sample (90%, 225 μl) was used for immunoprecipitation with the S9.6 antibody (Sigma), and the remaining fraction (10%, 25 μl) was used as the input control. Protein A beads (Dynabeads Protein A, Invitrogen) (100 μl) were blocked with 0.5% BSA in PBS containing 5 mM EDTA overnight at 4° C. After washing twice with DRIP buffer (50 mM Tris-HCl pH 7.4, 150 mM NaCl, 5 mM EDTA, 1% NP-40, 0.1% sodium deoxycholate), 20 g of S9.6 antibody (Sigma) or control mouse IgG (sc-2025, Santa Cruz) were applied to the blocked Dynabeads in DRIP buffer overnight at 4° C. After washing twice with DRIP buffer, the beads were resuspended in 100 μl of DRIP buffer containing 500 U of rNasin Plus inhibitor (Promega). Sonicated DNA was diluted to half concentration in a buffer containing 100 mM Tris-HCl pH 7.4, 10 mM EDTA, 2% NP-40, and 0.2% sodium deoxycholate. Then, 100 μl of the beads were added to DNA solution and incubated overnight at 4° C. with rotation. The beads were washed by the following steps: (1) twice with DRIP buffer; (2) twice with DRIP high buffer (50 mM Tris-HCl, pH 7.4, 500 mM NaCl, 5 mM EDTA, 1% NP-40, 0.1% sodium deoxycholate); (3) twice with DRIP Li buffer (50 mM Tris-HCl, pH 8.0, 250 mM LiCl, 1 mM EDTA, 0.5% NP-40, 0.5% sodium deoxycholate); (4) once with DRIP TE NaCl+ buffer (100 mM Tris-HCl pH8.0, 10 mM EDTA, 50 mM NaCl); and (5) once with DRIP TE buffer (100 mM Tris-HCl pH8.0, 10 mM EDTA). After removal of DRIP TE buffer using a magnetic stand, RNA:DNA hybrids were eluted from the beads in 200 μl of DRIP elution Buffer (50 mM Tris-HCl, pH 8.0, 10 mM EDTA, 1% sodium dodecyl sulfate (SDS)) by shaking at 65° C. for 30 min at 1400 r.p.m. (Benchmark Scientific, MultiTherm shaker H5000-H). The beads were removed from the supernatant by another round of separation with a magnetic stand and centrifugation. The supernatant was treated with 80 g of proteinase K (Roche) in the presence of 160 U of rNasin Plus inhibitor for 30 min at 42° C. RNA:DNA hybrids were purified using QIAquick Nucleotide Removal Kit (Qiagen) and eluted in 200 μl of buffer containing 5 mM Tris-HCl pH 8.5. For rNase H treatment, 50 μg of sonicated genomic DNA was preincubated with 25 U of E. coli-rNase H (NEB) overnight at 37° C. Recovery of RNA:DNA hybrids and rNase H treatment were evaluated by quantitative PCR analysis of spike RNA:DNA duplex.

qPCR Analysis of DRIP Products

Two microliters of DRIP products were used for qPCR with Power SYBR Green PCR Master Mix (Thermo Fisher Scientific) and QuantStudio 6 Flex Real-Time PCR System (Applied Biosystems, QuantStudio Real-Time PCR software).

### Dot Blot Analysis of DRIP Products

DRIP products or whole genomic DNA (10 μl) were mixed with 15 μl of 0.13 N NaOH/3.3 mM EDTA solution and incubated at 90° C. for 10 min. The denatured DRIP products were diluted in 100 μl of 6×SSC and spotted onto a Hybond N+ using Bio-Dot Apparatus. The membrane was cross-linked with UV (0.24 J) and pre-hybridized with ULTRAhyb Ultrasensitive Hybridization Buffer (Invitrogen) overnight at 42° C. 5′-³²P-labeled DNA or LNA-oligonucleotide probe was added to the hybridization buffer and incubated overnight at 42° C. The membrane was washed three times with 2×SSC/0.1% SDS solution for 15 min at 42° C. or 50° C. A fraction of DRIP samples (5%) was spotted as an input control onto the membrane. Hybridized probe signals were detected using Typhoon RGB Imager (GE Healthcare, Amersham Typhoon Control software). Consensus α-satellite, Alu, and LINE1 probes were hybridized and washed at 42° C. Using these less stringent hybridization and washing conditions, these probes target variations known to exist within sub-family members of each repetitive element. In particular, the Alu consensus probe is 44 nucleotides, so it can recognize all Alu subfamilies, except Alu that is missing the 3′ region. Therefore, consensus Alu and LINE1 probes recognize ˜11% and 18% of the human genome, respectively.

### RNA Strand Analysis of DRIP Products

Fifty micrograms of genomic DNA prepared as described above was diluted in 250 μl of sonication buffer and sonicated using Sonicator W-220 (20 cycles at Lv3.5, 10 s ON/40 s OFF). During sonication, samples were kept cold very carefully. A fraction of the genomic DNA sample (90%, 225 μl) was used for immunoprecipitation with the S9.6 antibody (Sigma or Kerafast), and the remaining fraction (10%, 25 μl) was used as the input control. Protein A beads (Dynabeads Protein A, Invitrogen) (100 μl) were blocked with 0.5% BSA in PBS containing 5 mM EDTA overnight at 4° C. After washing twice with DRIP buffer, 20 μg of 59.6 antibody or control mouse IgG (sc-2025, Santa Cruz) was applied to the blocked Dynabeads in DRIP buffer overnight at 4° C. After washing twice with DRIP buffer, the beads were resuspended in 100 μl of DRIP buffer containing 500 U of rNasin Plus inhibitor (Promega). Sonicated DNA was diluted to half concentration in a buffer containing 100 mM Tris-HCl pH 7.4, 10 mM EDTA, 2% NP-40, and 0.2% sodium deoxycholate. Then, 100 μl of the beads was added to the DNA solution and incubated overnight at 4° C. with rotation. The beads were washed by the following steps: (1) twice with DRIP buffer; (2) twice with DRIP High buffer; (3) twice with DRIP Li buffer; (4) one with DRIP TE NaCl+ buffer; and (5) twice with 100 mM Tris-HCl pH 8.0 buffer. After removal of 100 mM Tris-HCl pH 8.0 buffer using a magnetic stand, the beads were treated with 10 U of TURBO dNase (Thermo Fisher Scientific) at 37° C. for 1 h in 100 μl of TURBO dNase buffer containing 160 U of rNasin Plus inhibitor. After adding 100 μl of double concentration DRIP elution buffer (100 mM Tris-HCl, pH 8.0, 20 mM EDTA, 2% SDS), RNAs were eluted from the beads by shaking at 65° C. for 30 min at 1400 r.p.m. (Benchmark Scientific, MultiTherm shaker H5000-H). The beads were removed from the supernatant by another round of separation with a magnetic stand and centrifugation. The supernatant was treated with 80 g of proteinase K (Roche) for 30 min at 42° C. RNAs were purified using QIAquick Nucleotide Removal Kit (Qiagen) and eluted in 100 μl of buffer containing 5 mM Tris-HCl pH 7.6. For rNase H treatment, 50 μg of sonicated genomic DNA was preincubated with 25 U of E. coli-rNase H (NEB) overnight at 37° C. The input control was treated with rNase I and TURBO dNase and was purified using QIAquick Nucleotide Removal Kit.

DRIP products were incubated at 80° C. for 10 min. The denatured DRIP products were diluted in 100 μl of 6×SSC and spotted onto a Hybond N+ using Bio-Dot Apparatus. The membrane was cross-linked with UV (0.24 J) and pre-hybridized with ULTRAhyb Ultrasensitive Hybridization Buffer (Invitrogen) overnight at 42° C. 5′-³²P-labeled LNA-oligonucleotide probe was added to hybridization buffer and incubated overnight at 42° C. The membrane was washed three times with 2×SSC/0.1% SDS solution for 15 min at 42° C. or 55° C. Hybridized probe signals were detected using a Typhoon RGB Imager (GE Healthcare, Amersham Typhoon Control software). A fraction of DRIP samples (5%) was spotted onto the membrane.

Preparation of duplex substrates Sense or antisense oligonucleotides of telomere sequences were purchased from IDT and Dharmacon. The 5′ ends of RNA and DNA strands to be analyzed were biotinylated. Sense and antisense oligonucleotides were annealed in annealing buffer (10 mM Tris-HCl pH 7.5, 50 mM NaCl) to prepare perfectly matched or mismatched dsRNAs or RNA:DNA hybrids, which were used as substrates for in vitro editing assay.

### Preparation of Recombinant ADAR1 Proteins

All procedures were carried out at 4° C. HAT-ADAR1p110-WT-, FLAG-ADAR1p110-WT-, or HA-ADAR1p110-EAA-expressing Sf9 cells were prepared with baculovirus⁶⁹. The cells were washed with PBS and resuspended in Tris+ buffer (250 mM Tris pH 7.8, 1 mM dithiothreitol (DTT), 0.6 mM phenylmethylsulfonyl fluoride (PMSF), proteinase inhibitor cocktail). The cells were sonicated and debris was removed by centrifugation. The supernatant (cell extract) was diluted with an equal volume of 2×TGK buffer (100 mM Tris-HCl pH 7.8, 200 mM NaCl, 40% glycerol, 1 mM DTT, 0.6 mM PMSF, proteinase inhibitor cocktail) and stored at −80° C.

HAT-ADAR1p110-WT was purified using TALON Metal Affinity Resin (Clontech). The resin was prewashed with STD300 buffer (50 mM Tris-HCl pH 7.0, 300 mM NaCl, 20% glycerol, 1 mM β-mercaptoethanol, 0.05% NP-40). After buffer exchange to STD300 using Zeba™ 7 K molecular weight cut-off (MWCO) spin desalting column (Thermo Fisher), the cell extract was loaded onto the resin. After washing with STD300 buffer, the resin was treated with 80 kU of micrococcal nuclease (NEB) for 30 min in STD300 buffer containing 1 mM CaCl₂) at room temperature and washed with STD300 buffer containing 0.5 mM EDTA and 0.5 mM EGTA and then washed with STD300 buffer containing 7.5 mM imidazole. HAT-ADAR1p110-WT recombinant protein was eluted with STD300 buffer containing 150 mM imidazole and proteinase inhibitor cocktail. Imidazole was removed by using Zeba™ 7 K MWCO spin desalting column.

FLAG-ADAR1p110-WT or HA-ADAR1p110-EAA was purified using anti-FLAG M2 agarose (Sigma) or anti-HA agarose (Thermo Fisher Scientific), respectively. The agarose was washed with STD150 buffer (50 mM Tris-HCl pH 7.0, 150 mM NaCl, 20% glycerol, 1 mM β-mercaptoethanol, 0.05% NP-40). After buffer exchange to STD150 using Zeba™ 7 K MWCO spin desalting column, the cell extract was loaded onto the agarose. After washing with STD150 buffer, the agarose was treated with 80 kU of micrococcal nuclease (NEB) for 30 min in STD150 buffer containing 2 mM CaCl₂) at room temperature and washed with STD150 buffer containing 3 mM EDTA and 3 mM EGTA, STD150 buffer, STD500 buffer (50 mM Tris-HCl pH 7.0, 500 mM NaCl, 20% glycerol, 1 mM β-mercaptoethanol, 0.05% NP-40), and again STD150 buffer. FLAG-ADAR1p110-WT or HA-ADAR1p110-EAA recombinant protein was eluted with STD150 buffer containing protease inhibitor cocktail and 0.1 mg/ml FLAG peptide or HA peptide, respectively.

All recombinant proteins purified were stored in STD150 buffer containing 1 mM DTT, instead of 1 mM β-mercaptoethanol, at −80° C.

### In Vitro Editing Assay

The in vitro editing reaction mixture, containing 5 nM of telomere RNA:RNA duplex substrates and 75 nM of HAT-ADAR1p110-WT, FLAG-ADAR1p110-WT, or HA-ADAR1p110-EAA protein, was incubated at 37° C. for 2 h in in vitro editing buffer I (20 mM HEPES-KOH pH 7.5, 100 mM NaCl, 0.01% NP-40, 5% glycerol, 1 mM DTT). For editing of RNA:DNA hybrid substrates, in vitro editing buffer II (20 mM HEPES-KOH pH 7.5, 20 mM NaCl, 0.01% NP-40, 5% glycerol, 1 mM DTT) was used. Edited RNA or DNA strands were purified using Dynabeads MyOne Streptavidin C1 (Thermo Fisher Scientific). To remove opposite RNA or DNA strands, rNase H (NEB) or TURBO dNase (Thermo Fisher Scientific) was used, respectively. For sequencing of edited substrates, reverse transcription-PCR was carried out for RNA strands, while PCR was carried out for DNA strands. RT reactions were carried out using SuperScript III Reverse Transcriptase (Thermo Fisher Scientific), and PCR reactions were performed using Platinum Taq DNA polymerase (Thermo Fisher Scientific). PCR products were sequenced using a specific sequencing primer, and the ratio of A and G peaks in the chromatograms were analyzed by CodonCode Aligner (CodonCode Corporation).

### Protein Co-IP

Cells expressing FLAG-ADAR1p110-WT, FLAG-ADAR1p110-EAA, FLAG-ADAR2-WT, FLAG-ADAR2-EAA, or FLAG-rNaseH2A were fixed with 0.3% formaldehyde in PBS containing 1 mM DTT at room temperature for 10 min. After washing twice with PBS, the cells were suspended in co-IP buffer (50 mM Tris-HCl pH 7.6, 150 mM NaCl, 1 mM EDTA, 1% NP-40, 0.5% sodium deoxycholate, 0.1% SDS, proteinase inhibitor cocktail, PhosStop, rNasin Plus inhibitor) and sonicated. The debris was removed by centrifugation and the supernatant was incubated overnight at 4° C. with 50 μl of anti-FLAG M2 magnetic beads (Sigma), prewashed and blocked with 20% BSA blocker in co-IP buffer. The beads were washed three times with co-IP buffer and NP-40 wash buffer (50 mM Tris-HCl pH 7.6, 150 mM NaCl, 1 mM EDTA, 1% NP-40). Laemmli buffer containing proteinase inhibitor cocktail and PhosStop phosphatase inhibitor cocktail was added to the beads and boiled at 98° C. for 10 min. Interacting proteins with ADAR1p110-WT, ADAR2, or rNaseH2A were detected by immunoblot analysis as described above; 2.5% extracts of co-IP products were used as input controls.

Preparation of rNase H2 Complex and RNA:DNA Hybrid Cleavage Assay

To prepare recombinant human rNase H2A/2B/2C triple complex, pET28-FLAG-rNaseH2A and pET15-His-rNaseH2B/2C vectors were co-transformed into E. coli BL21 cells cultured in LB medium containing 100 μg/ml ampicillin and 20 μg/ml kanamycin. Protein induction was started at an optical density of 0.6 with 0.4 mM isopropyl β-D-1-thiogalactopyranoside and incubated overnight at 30° C. After harvesting cells by centrifugation, cells were suspended in 40 mM HEPES pH 7.0, 75 mM NaCl, 5% glycerol, proteinase inhibitor cocktail, and 1 mg/ml lysozyme and then sonicated. The debris was removed by centrifugation twice at 12,000×g. The supernatant was diluted with an equal volume of dilution buffer (40 mM NaH₂PO₄ pH 7.0, 500 mM NaCl, 5% glycerol) and mixed with Ni-NTA agarose (Qiagen) at 4° C. The agarose was washed with STD300 buffer containing 5 mM imidazole. Recombinant rNase H2A/2B/2C complex was eluted with 20 ml of STD300 buffer containing 150 mM imidazole and proteinase inhibitor cocktail. Anti-FLAG M2 affinity gel (Sigma) was washed with STD150 buffer and mixed with an eluted fraction of Ni-NTA agarose at 4° C. After washing with STD150 buffer, the resin was treated with 80 kU of micrococcal nuclease (NEB) for 30 min in STD150 buffer containing 2 mM CaCl₂) at room temperature and washed with STD500 buffer and then with STD150 buffer. FLAG-rNase H2A/His-rNase H2B/2C complexes were eluted with 0.1 mg/ml FLAG peptide in STD150 buffer containing proteinase inhibitor cocktail.

5′-³²P-labeled oligonucleotide RNAs were annealed with complementary DNAs as described above. Cleavage assays of RNA:DNA hybrid by rNase H2A/2B/2C complex were done in a 50 μl reaction mixture containing 50 mM Tris-HCl pH 8.5, 75 mM KCl, 3 mM MgCl₂, 10 mM DTT, 2 nM rNase H2A/2B/2C complex, 1 nM RNA:DNA substrate, and rNasin plus inhibitor. Cleavage assays by recombinant human rNase H1 (ab153634, Abcam) were done in a 50 μl reaction mixture containing 25 mM Tris-HCl pH 7.5, 50 mM KCl, 5 mM MgCl₂, 1 mM DTT, 10 μg/ml BSA, 5 nM rNase H1, 1 nM RNA:DNA substrate, and rNasin plus inhibitor. Reaction mixtures were incubated at 37° C. and 7.5 μl aliquots were taken after 0, 5, 10, 30, and 60 min. At each time point, to stop the reaction, gel loading buffer (80% formamide, 20% glycerol, 0.025% bromophenol) was added to the aliquots. After heating at 80° C. for 10 min, samples were analyzed by 10% Urea-PAGE. 5′-³²P-labeled RNA signals were detected using a Typhoon RGB Imager (GE Healthcare, Amersham Typhoon Control software).

### Telomere FISH Analysis

Exponentially growing cells were treated with colcemid (60 ng/ml) for 1 h and harvested. Then, cells were subsequently swollen in a hypotonic 0.075 M KCl solution for 20 min at room temperature and then fixed in a freshly prepared 3:1 mix of methanol:acetic acid four times. After fixation, cells were dropped onto glass microscope slides and allowed to dry for 2 days at room temperature. The slides were immersed in PBS at 37° C. for 30 min, fixed in 4% formaldehyde in PBS for 2 min, and washed three times with PBS for 5 min. The slides were then treated with 1 mg/ml pepsin solution (pH 2.0) at 37° C. for 2-5 min. After washing with PBS for 10 s, the slides were fixed in 4% formaldehyde in PBS and washed three times with PBS for 5 min. Then, 10 μl of hybridization mixture containing 70% formamide, 1% (w/v) blocking reagent (Roche) in a maleic acid buffer (pH 7.0), and 3 ng of fluorescence-labeled telomeric peptide nucleic acid (PNA) probe FITC-(CCCTAA)₄ were applied to each slide and mounted under a coverslip. The slides were heated on an aluminum heat block at 80° C. for 3 min and hybridized with PNA probe for 5 h in 37° C. After hybridization, the slides were washed twice in 70% formamide/10 mM Tris (pH 7.2) for 15 min, followed by washing three times with 50 mM Tris/150 mM NaCl (pH 7.5)/0.05% Tween-20. Finally, DNA was counterstained with Vectashield with DAPI (Vector Lab). The chromosome samples were observed using a fluorescence microscope and digital images were recorded using a CCD camera and LAS X software (Leica).

### Immuno-FISH Assay for MEF Cells

Cells were seeded onto coverslips and cultured overnight. The adhered cells were washed twice with cold PBS for 5 min and fixed with 4% paraformaldehyde in PBS for 10 min at room temperature. Cells were then washed three times with PBS for 5 min each and permeabilized with ice-cold 0.5% NP-40 in PBS for 10 min on ice. Cells were washed three times with PBS for 5 min each and incubated with anti-phosphorylated histone γH2AX antibody (Millipore), followed by Alexa 488 secondary antibody (Molecular Probes) with 30% Blocker BSA (bovine serum albumin) buffer (Thermo Fisher Scientific). After staining, labeled protein was cross-linked with 4% paraformaldehyde in PBS for 20 min at room temperature. The samples were washed two times with PBS for 5 min and then dehydrated in 70, 90, and 100% ethanol for 3 min each and air-dried. Hybridization mixtures (10 μl) containing 3 ng of fluorescence-labeled telomeric PNA probe were applied to the slide and mounted under a coverslip. The slides were heated for 3 min on a hot plate at 80° C. After hybridization with a telomeric PNA probe for 5 h, the cells were washed three times with 70% formamide/10 mM Tris (pH 6.8) for 15 min, followed by a 5-min wash with 0.05 M Tris/0.15 M NaCl (pH 7.5)/0.05% Tween-20 and a 5-min wash with PBS. Mounting and microscopic analysis was performed as for the telomere FISH analysis.

### Immuno-FISH Assay for HeLa Cells

Cells were seeded onto collagen-coated Ibidi 3-well chambers and transfected with siControl or siADAR1-1. The adhered cells were washed twice with PBS and fixed with 4% paraformaldehyde (Thermo Fisher Scientific) in PBS at room temperature for 10 min. After washing twice with PBS, the cells were permeabilized with 0.25% Tween-20 in PBS. After washing twice with PBS, the cells were treated with 20 g of rNase A and 500 U of rNase I in PBS at 37° C. for 1 h. After washing three times with PBS, the cells were blocked with 30% Blocker BSA buffer (Thermo Fisher Scientific) for 30 min and incubated with anti-γH2AX antibody (Abcam), followed by Alexa 488 or 647 secondary antibody (Thermo Fisher Scientific) with 30% Blocker BSA buffer (Thermo Fisher Scientific). After staining, labeled protein was cross-linked with 4% paraformaldehyde in PBS at room temperature for 10 min. After washing twice with PBS, the cells were dehydrated in 70%, 85%, and 100% ethanol for 2 min each and air-dried. Fifty microliters of hybridization mixture containing 500 nM TelC-Cy3 PNA probe (PNA bio), 20 mM Tris-HCl pH 7.4, 60% formamide, and 0.5% blocking reagent (Roche) was applied to the chamber. The chamber was heated for 3 min on a heat block at 80° C. After hybridization with TelC-Cy3 PNA probe at room temperature for 5 h, the cells were washed three times with PBS at 45° C. for 10 min. Finally, DNA was counterstained with DAPI. The slides were mounted with ProLong Gold (Thermo Fisher Scientific). Microscopic images were obtained by using a Leica TCS SP5 DMI6000 CS Confocal Microscope (Leica). Fluorescent images were captured with a ×63.0 lens by LAS X software (Leica). Ectopic expression of FLAG-ADAR1p110 was evaluated by immunostaining with anti-FLAG M2 antibody.

### Chromosome Orientation-FISH

Cells were cultured in a medium containing a 3:1 ratio of 5′-bromo-2′-deoxyuridine (BrdU, Sigma):5′-bromo-2′-deoxycytidine (BrdC, Sigma) at a total final concentration of 10 μM during the final 24 h. Colcemid addition led to the accumulation of mitotic cells. Cultures were trypsinized and then treated with hypotonic KCl, fixed, and dropped onto microscope slides. Prior to hybridization of the single-stranded telomere probe (as above for FISH), slides were treated with 0.5 mg/ml rNase A (Sigma) for 10 min at 37° C. and then stained with 0.5 g/ml Hoechst 33258 (Sigma) in 2×SSC for 15 min at room temperature. Slides were then exposed to 365 nm UV light for 25 min. The BrdU/BrdC-substituted DNA strands were digested with 3 U/μl of exonuclease III (Promega) in a buffer supplied by the manufacturer (50 mM Tris-HCl, 5 mM MgCl₂, and 5 mM dithiothreitol, pH 8.0) for 10 min at room temperature. An additional denaturation was performed in 70% formamide, 2×SSC at 70° C. for 1 min, followed by dehydration in a cold ethanol series (70, 85, and 100%). The CO-FISH procedure results in the original parental strands serving as single-stranded chromosomal target DNA for hybridization of single-stranded probes.

### Time-Lapse Imaging

HeLa cells were treated with CellLight Tubulin-GFP and BacMam 2.0 (Thermo Fisher Scientific) for 12 h before siRNA transfection. Transfection of siRNAs into HeLa cells at 1 nM concentration was carried out as described above. Nuclei were visualized by staining of DNA with SiR-DNA reagent (Cytoskeleton) (0.25 μM) for 6 h. Cells were cultured in CellView 3.5 cm glass-bottomed dishes (Greiner). Time-lapse images were obtained using a Leica TCS SP5 DMI6000 CS Confocal Microscope between 48 and 72 h post transfection.

### Statistics and Reproducibility

All experiments were performed at least twice or more independent times with similar results. Image quantitation was done using Image J or ImageQuant software (GE Healthcare).

Data were analyzed using Microsoft Excel (Microsoft Corporation) and were presented as means±SD or SEM. Two-tailed t tests were conducted where the minimum level of significance was P<0.05.

### Example 2: ADAR1 RNA Editing Enzyme Regulates R-Loop Formation and Genome Stability at Telomeres in Cancer Cells

ADAR1 is involved in adenosine-to-inosine RNA editing. The cytoplasmic ADAR1p150 edits 3′UTR double-stranded RNAs and thereby suppresses induction of interferons. Loss of this ADAR1p150 function underlies the embryonic lethality of Adar1 null mice, pathogenesis of the severe autoimmune disease Aicardi-Goutieres syndrome, and the resistance developed in cancers to immune checkpoint blockade. In contrast, the biological functions of the nuclear-localized ADAR1p110 remain largely unknown. Here, we report that ADAR1p110 regulates R-loop formation and genome stability at telomeres in cancer cells carrying non-canonical variants of telomeric repeats. ADAR1p110 edits the A-C mismatches within RNA:DNA hybrids formed between canonical and non-canonical variant repeats. Editing of A-C mismatches to I:C matched pairs facilitates resolution of telomeric R-loops by rNase H2. This ADAR1p110-dependent control of telomeric R-loops is required for continued proliferation of telomerase-reactivated cancer cells, revealing the pro-oncogenic nature of ADAR1p110 and identifying ADAR1 as a promising therapeutic target of telomerase positive cancers.

Adenosine deaminase acting on RNA (ADAR) is the enzyme involved in adenosine-to-inosine RNA editing (A-to-I RNA editing), and three ADAR gene family members (ADAR1, ADAR2, and ADAR3) have been identified in vertebrates¹⁻⁵. ADARs share common domain structures, such as multiple dsRNA-binding domains (dsRBDs) and a separate catalytic domain^{6,7}. Both ADAR1 (ADAR, DRADA) and ADAR2 (ADARB1) are catalytically active enzymes, whereas no catalytic activity of ADAR3 (ADARB2) has been shown so far¹⁴. A-to-I editing occurs most frequently in noncoding regions that contain repetitive elements Alu and LINE^{8,9}, and many millions of editing sites have been identified in the human transcriptome of these repetitive sequences⁹⁻¹¹.

Two ADAR1 isoforms, p150 and p110, are generated by the use of separate promoters and alternate splicing¹². ADAR1p150 is mostly in the cytoplasm, whereas ADAR1p110 mainly localizes in the nucleus¹³. The cytoplasmic ADAR1p150 regulates the dsRNA-sensing mechanism mediated by melanoma-differentiation-associated protein 5 (MDA5), mitochondrial antiviral signaling protein (MAVS), and interferon signaling (MDA5-MAVS-IFN signaling)¹⁴⁻¹⁶. The cytoplasmic ADAR1p150 edits 3′-untranslated region (3′-UTR) dsRNAs primarily comprising inverted Alu repeats and thereby suppresses activation of MDA5-MAVS-IFN signaling¹⁴⁻¹⁶. This ADAR1p150 function in the regulation of the MDA5-MAVS-IFN pathway underlies the embryonic lethality of Adar1-null mice^{17,18} and also the pathogenesis of Aicardi-Goutieres syndrome (AGS; AGS1-7 subgroups known), a severe human autoimmune disease against endogenous nucleic acids¹⁴⁻¹⁶. Mutations of seven genes, including rNaseH2A (AGS4), rNaseH2B (AGS2), rNaseH2C (AGS3), and ADAR1 (AGS6), have been identified in association with AGS, and ten AGS6 mutations of ADAR1 have been reported so far¹⁹. Finally, this ADAR1p150-mediated suppression of IFN signaling also represses tumor responsiveness to immune checkpoint blockade²⁰, revealing the pro-oncogenic ADAR1p150 function. Analysis of The Cancer Genome Atlas database revealed elevated ADAR1 expression and A-to-I editing levels in almost all types of cancers^{21,22}, indicating that this pro-oncogenic ADAR1p150 function helps cancer cells suppress inflammatory responses and thus avoid host immunosurveillance²⁰. In contrast to the recent advance in the knowledge of ADAR1p150 functions, the biological functions of the nuclear-localized ADAR1p110, other than its involvement in editing of intronic Alu dsRNAs²³, have remained mostly unknown.

Newly transcribed RNA usually dissociates from its template DNA strand immediately after transcription, but occasionally it forms a stable RNA:DNA hybrid, which consequently leaves the sense DNA in a single-stranded form. This structure, called an R-loop, often spans 100-2000 bp and causes abortive transcription and instability of the genome^{24,25}. Several mechanisms are known to suppress the formation of R-loops, for example, degradation of RNA strands of RNA:DNA hybrids by rNase H1²⁶ and rNase H2^{27,28} and unwinding of RNA:DNA hybrids by helicases such as dExH-box helicase 9 (DHX9)²⁹ and senataxin (SETX)³⁰. Human diseases including amyotrophic lateral sclerosis type 4, ataxia-ocular apraxia type 2, and AGS are caused by the accumulation of R-loops due to deficiency in one of those suppression mechanisms^{31,32}. Telomere end regions consisting of repetitive sequences are important for protection of these regions from recombination and degradation^{33,34}. However, telomeric repeat regions are also known to be naturally prone to the formation of R-loops^{24,25}, which in turn causes telomere instability and perhaps underlies carcinogenesis of certain cancers^{31,36} The canonical hexameric repeat sequence of the telomeric G-strand (sense strand) is TTAGGG^{33,37} Interestingly, detection of widespread telomeric variant repeats such as TCAGGG and TTGGGG has been reported in cancer cells³⁸⁻⁴⁰. Mutations/variations (nucleotides) of telomeric canonical repeat DNA sequence TTAGGG (antisense sequence CCCTAA) detected in cancer cells such as TTGGGG (CCCCAA) and TCAGGG (CCCTGA) are emphasized by underlining. Their RNA sequence versions are UUGGGG (CCCCAA) and UCAGGG (CCCUGA). In addition, adenosine residues to be edited by ADAR1 were also emphasized by underlining: TTAGGG (RNA sequence UUAGGG).

In this study, we found an important role for the ADAR1p110 isoform in resolution specifically of the R-loops formed at telomeric repeat regions. ADAR1p110 edits both the RNA and the DNA strands of telomeric repeat RNA:DNA hybrids containing mismatched base pairs formed between canonical and variant repeats. ADAR1p110-mediated editing of A-C-mismatched base pairs, which converts them to I:C-matched base pairs, is required for degradation of the RNA strands of telomeric repeat RNA:DNA hybrids by rNase H2. We found that rNase H2 is incapable of resolving mismatch-containing telomeric RNA:DNA hybrids by itself. This newly found ADAR1p110 role in suppression of telomeric R-loops seems to be essential for the continued proliferation of telomerase-reactivated cancer cells with accumulated variant telomeric repeats, revealing yet another pro-oncogenic function of ADAR1.

### Depletion of ADAR1 Results in Telomere DNA Damage, Telomere Abnormalities, and Mitotic Arrest

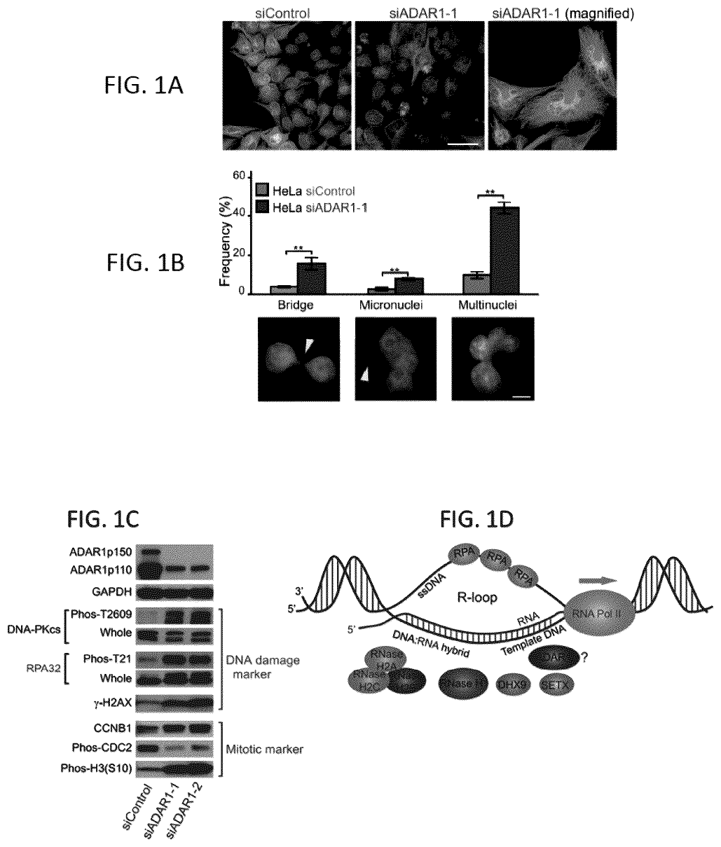

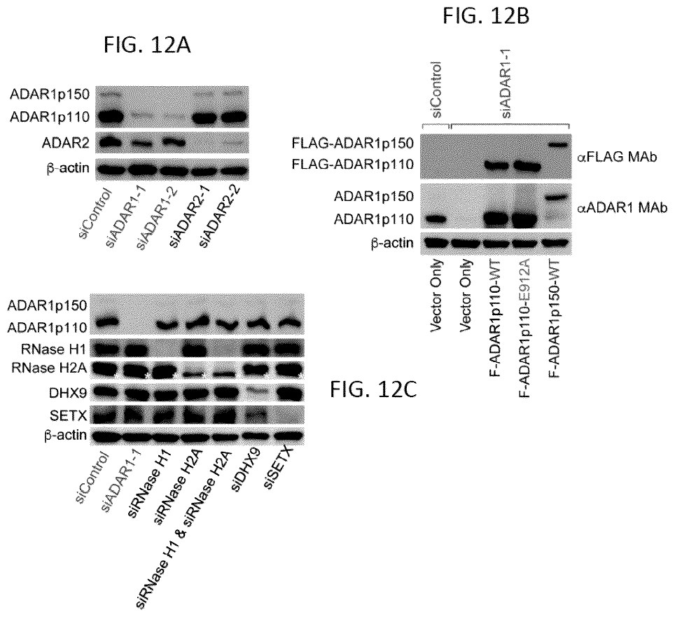

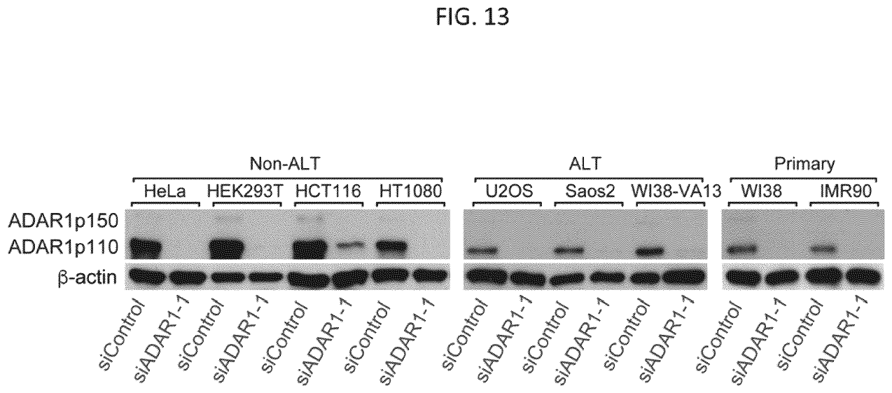

We recently made several observations that indicated the involvement of ADAR1 in the maintenance of telomere stability and mitosis. First, significantly increased telomere abnormality, such as telomere fusions, was detected with Adar1-null mouse embryonic fibroblast (MEF) cells derived from Adar1-null mouse embryos¹⁸. In contrast, no significant telomere abnormality was detected with Adar2-null MEF cells derived from Adar2-null mouse embryos⁴¹. Chromosome orientation fluorescence in situ hybridization (CO-FISH) analysis revealed significantly increased involvement of leading strands in telomere fusions detected in Adar1-null MEF cells, indicating that ADAR1 is involved in the mechanism that maintains the integrity of the telomere leading strands. Detection of significantly increased telomere dysfunction-induced foci (TIF) revealed by telomere FISH and γH2AX immunostaining suggested accumulation of DNA damage, if not exclusively, mainly at telomeres in Adar1-null MEF cells, which must be closely related to the increased telomere fusions. Second, time-lapse imaging of HeLa cells undergoing ADAR1 gene knockdown revealed many aberrantly shaped nuclei that appeared to be arrested during mitosis, and these aberrant cells eventually died, most likely by apoptosis. Close examination revealed the presence of increased bridged nuclei, micronuclei, and multinuclei in ADAR1-depleted cells (FIG. 1B and FIG. 12A). Significantly increased TIFs were detected also in ADAR1-depleted HeLa cells. Ectopic expression of ADAR1p110-WT but not ADAR1-E912A, a mutant without catalytic activity https://www.ncbi.nlm.nih.gov/omc/articles/PMC7955049/—CR6 (FIG. 12B), suppressed induction of TIFs in ADAR1-depleted cells (but not by ADAR1p110-E912A mutant nor by vector only), demonstrating the importance of ADAR1-mediated A-to-I editing activity in the maintenance of telomere stability. Western blotting analysis revealed significantly upregulated DNA damage markers such as phosphorylated DNA-dependent protein kinase catalytic subunit (DNA-PKcs), γH2AX, and phosphorylated RPA32 in ADAR1-depleted HeLa cells (FIG. 1C). In addition, elevated levels of CCNB1 and histone H3 phosphorylated at serine 10 as well as decreased expression of phosphorylated CDC2 indicated that ADAR1-depleted cells were arrested during mitosis (FIG. 1C). These results together suggest that ADAR1 deficiency causes aberrant mitotic arrest with extensive DNA damage at telomeres.

### Accumulation of R-Loops Specifically at Telomeric Repeat Regions in ADAR1-Depleted Cells

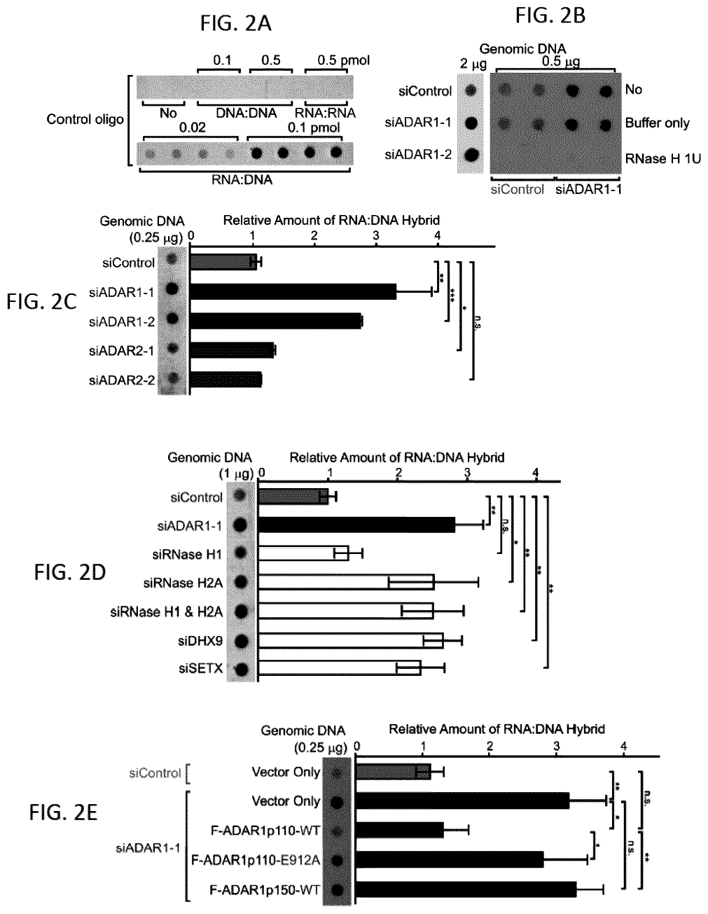

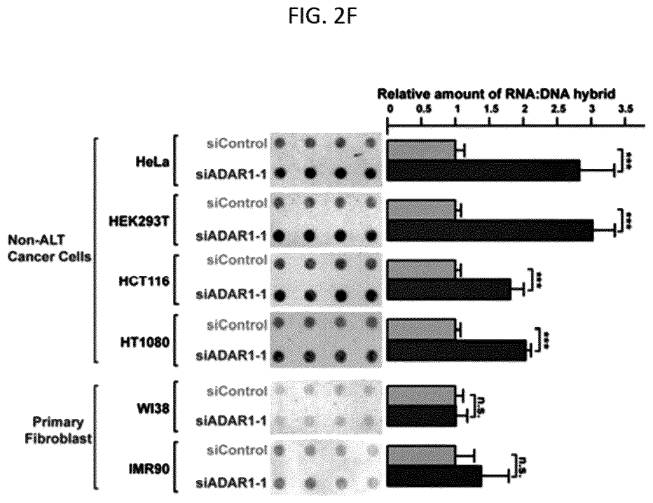

One of the DNA damage markers increased in ADAR1-depleted cells was replication protein A 32 kDa subunit (RPA32 or RPA2) (FIG. 1C). Phosphorylated RPA32 binds to single-stranded DNAs (ssDNAs) and, thus, is an effective marker for R-loops⁴² (FIG. 1D). We reasoned that ADAR1 depletion might result in the accumulation of R-loops, which could cause DNA damage, telomere abnormalities, and mitotic arrest⁴³⁻⁴⁵. Dot blot analysis using the S9.6 antibody specific to RNA:DNA hybrids⁴⁶ (FIG. 2A) revealed that ADAR1 depletion indeed resulted in significantly increased the formation of RNA:DNA hybrids (FIG. 2B and FIG. 12A). Treatment with Escherichia coli-rNase H, which digests RNA strands of RNA:DNA hybrids, eliminated dot blot signals, confirming specific detection of RNA:DNA hybrids (FIG. 2B). ADAR2 depletion had almost no effect on the formation of RNA:DNA hybrids (FIG. 2C and FIG. 12A), indicating that the R-loop regulatory function is specific to ADAR1.

rNase H1²⁶ and H2²⁸ degrade RNA strands of RNA:DNA hybrids, while DHX9²⁹ and SETX³⁰ unwind RNA:DNA hybrids: they all are capable of resolving already formed R-loop structures (FIG. 1D). Depletion of these R-loop regulators, either by single knockdown or combination knockdown of rNase H1 and rNase H2, except for rNase H1 single knockdown, resulted in significant accumulation of RNA:DNA hybrids in HeLa cells (FIG. 2D and FIG. 12C). Importantly, the amount of RNA:DNA hybrid formed in ADAR1-depleted cells was equivalent to or even more than those formed upon depletion of these known regulators (FIG. 2D). Using various ADAR1-expressing lentivirus systems, we conducted rescue experiments: repression of RNA:DNA hybrids formed in ADAR1-depleted HeLa cells. As expected, ectopic expression of FLAG-tagged ADAR1p110-WT (wild type) efficiently suppressed the formation of RNA:DNA hybrids (FIG. 2E). In contrast, the ADAR1p110-E912A could not rescue despite being expressed at a higher level than that of endogenous ADAR1p110 (FIG. 2E and FIG. 12B). Furthermore, ADAR1p150-WT did not suppress the formation of RNA:DNA hybrids (FIG. 2E and FIG. 12B). These results demonstrated that A-to-I editing activity of ADAR1p110, but not ADAR1p150, is required for suppression of R-loops. In addition, we found that ADAR1 depletion induced accumulation of R-loops only in non-ALT (telomerase reactivated) cancer cells but had no effects on ALT positive cancer cells or normal fibroblast cells (FIG. 2F)

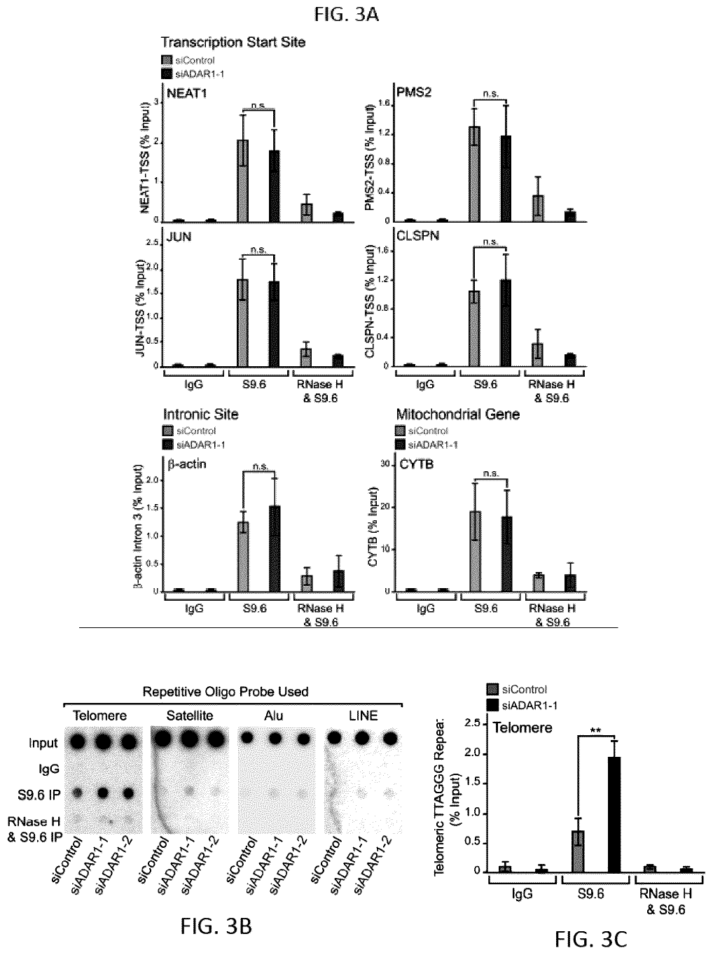

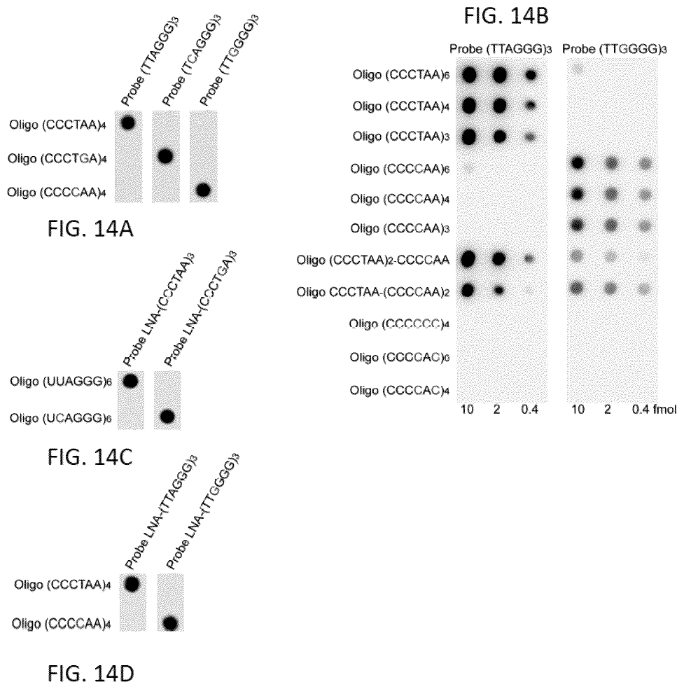

We next investigated where in the genome ADAR1p110 controls the formation of R-loops. It has been reported that certain genome loci such as actively transcribed genes and mitochondrial genes are particularly prone to the formation of R-loops⁴⁷⁻⁴⁹. Accordingly, we conducted quantitative PCR (qPCR) analysis of DNA:RNA immunoprecipitation (DRIP) products pulled down with the S9.6 antibody. The results revealed that ADAR1 depletion had no effects on the formation of R-loops at the known transcription start sites of NEAT1, JUN, PMS2, and CLSPN genes, an intronic site of the β-actin gene, and an exonic site of the mitochondrial gene CYTB⁴⁷⁻⁴⁹ (FIG. 3A). In addition to actively transcribed genes, centromeric and telomeric repeat regions have been reported to be prone to the formation of R-loops^{24,25,35}. The repetitive elements of retrotransposons such as Alu and LINE are known to be the most frequent targets for ADAR1^{9,50}. To this end, DRIP products were next tested for dot blot hybridization analysis using four different probes carrying telomeric repeat, α-satellite centromeric repeat, Alu, and LINE1 sequences (FIG. 14A, FIG. 14B). We found that ADAR1 depletion resulted in the accumulation of RNA:DNA hybrids specifically at telomeric repeat regions, but not at α-satellite centromeric repeats nor Alu and LINE1 repeats (FIG. 3B, FIG. 3C), which account for nearly 30% of the human genome⁵¹. Accumulation of R-loops at telomeres could explain the increased TIFs, various telomere abnormalities observed in Adar1-null MEF cells (FIG. 11A-FIG. 11E), and mitotic arrest detected in ADAR1-depleted HeLa cells (FIG. 1C). We conclude that telomere repeats are, if not exclusive, the major targets of ADAR1.

### ADAR1p110 Cannot Edit Completely Complementary Telomeric Repeat RNA:DNA Hybrids

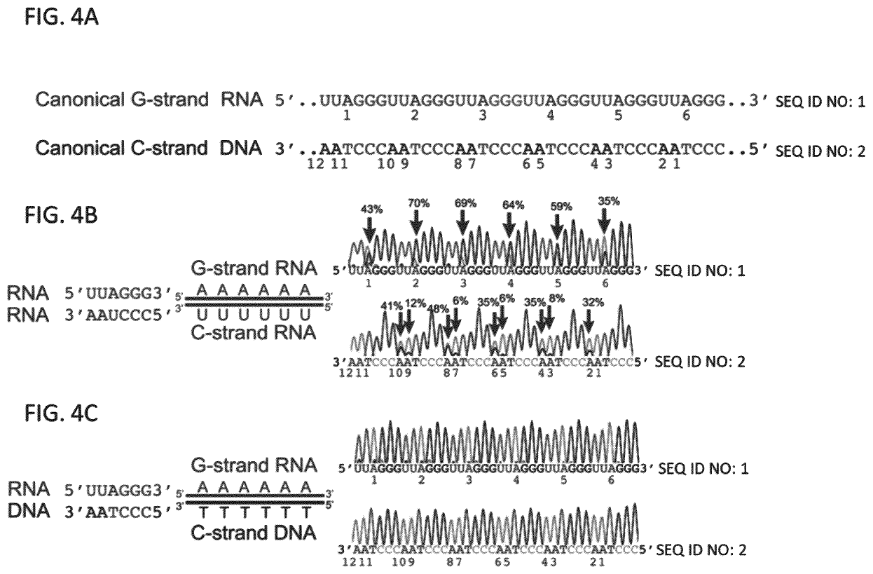

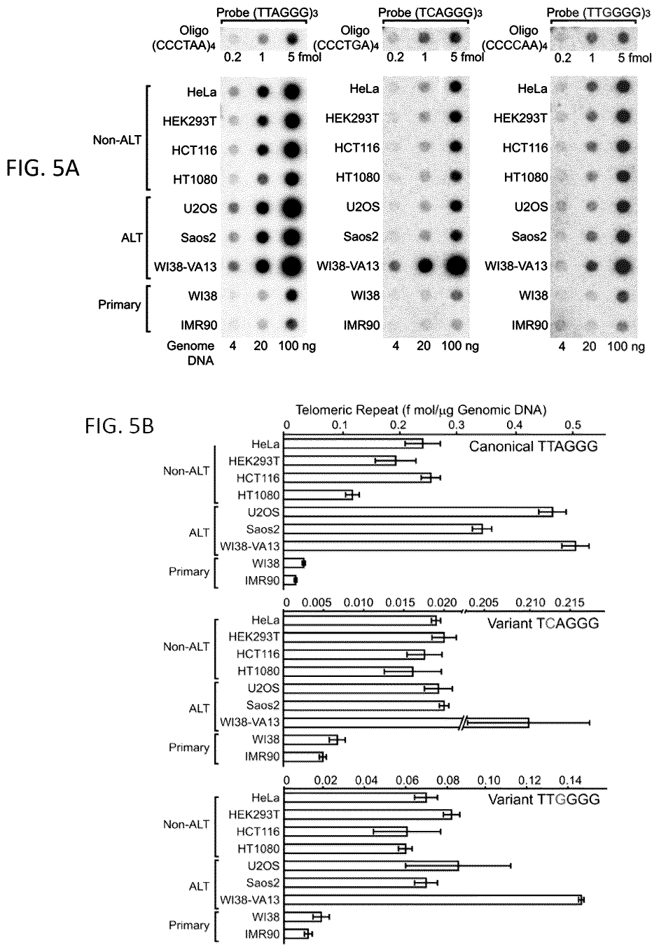

ADAR1 was originally identified as an A-to-I editing enzyme specific to dsRNAs^{52,53} However, recent studies by Beal and his colleagues⁵⁴ revealed that a catalytic domain fragment of ADAR1 can edit DNA strands of RNA:DNA hybrid substrates in vitro. However, it was not known whether the full-length ADAR1 also had such RNA:DNA editing activity and, if so, whether that RNA:DNA hybrid editing activity is significant in vivo. Interestingly, the adenosine within the hexameric TTAGGG (UUAGGG) sequence of canonical telomeric repeats (FIG. 4A) is the most favored A-to-I editing sites of ADAR1: it prefers 5′ nearest-neighbor U and 3′ nearest-neighbor G⁵⁵. Together with our results (FIG. 3B, FIG. 3C), we reasoned that ADAR1p110 might edit RNA:DNA hybrids carrying telomeric repeat sequences. Editing of A:U or A:T base pairs to I:U or I:T wobble base pairs within telomeric repeat RNA:DNA hybrids would reduce their thermodynamic stability, which in turn would facilitate their dissociation or unwinding by R-loop regulatory helicases such as DHX9 and SETX (FIG. 1D). Accordingly, we prepared telomeric repeat duplex substrates (RNA:RNA and RNA:DNA hybrid) consisting of perfectly complementary sense and antisense strands (FIG. 4B, FIG. 4C) and tested them for in vitro editing with ADAR1p110, the ADAR1 isoform relevant to R-loop regulation (FIG. 2E). As expected, ADAR1p110 very efficiently edited six adenosine residues of the G-strand RNA (FIG. 4B, top), and also, with lower efficiency, C-strand adenosines (FIG. 4B, bottom) of the completely complementary telomeric repeat dsRNA. In contrast, ADAR1p110 editing of the completely matched telomeric repeat RNA:DNA hybrids (FIG. 4C, top and bottom for G-strand RNA and C-strand DNA, respectively) was very inefficient, only at the background level, that is, <5%, revealing a clear difference in activity toward telomeric repeat dsRNAs versus RNA:DNA hybrids.

### Widespread Detection of Telomeric Variant Repeats in ALT and Non-ALT Cancer Cells

Variant telomeric repeats such as TCAGGG and TTGGGG are detected in the telomeres of cancer cells including HeLa cells. In some cases, the proportion of variant repeats almost equals that of canonical repeats^{38,39}. Cancer cells maintain telomere length either by the non-canonical telomere extension mechanism, known as the alternative lengthening of telomeres (ALT), in ALT-positive cancer cells (ALT cells) or by reactivating telomerase in ALT-negative cancer cells such as HeLa cells (non-ALT cells)^{33,37}. Telomeric variant repeats are amplified by homologous recombination between telomeric repeats in ALT cells, and also by a currently unknown mechanism in non-ALT cells³⁹. Using telomeric canonical TTAGGG and variant TCAGGG or TTGGGG repeat-specific probes capable of distinguishing a single-nucleotide mismatch (FIG. 14A, FIG. 14B), we detected indeed significant amounts of telomeric variant TCAGGG and TTGGGG repeats in four non-ALT cell lines, including HeLa cells, as well as three ALT cell lines, whereas the amount of telomeric variant repeats was much less in two primary fibroblast cell lines examined (FIG. 5A, FIG. 5B).

### Accumulation of RNA:DNA Hybrids Containing Telomeric Variant Repeats in ADAR1-Depleted Cells

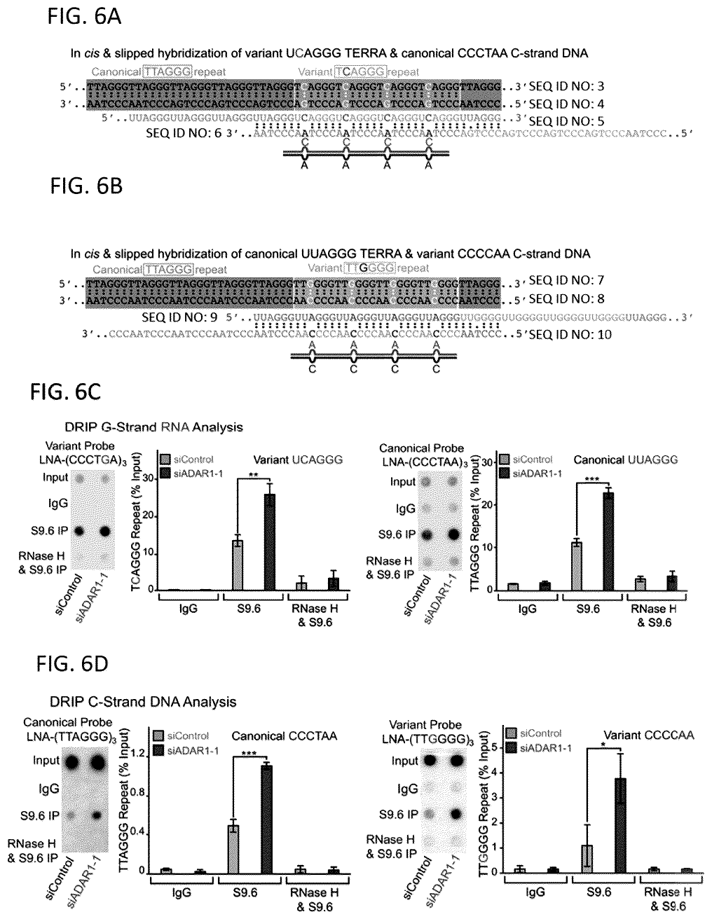

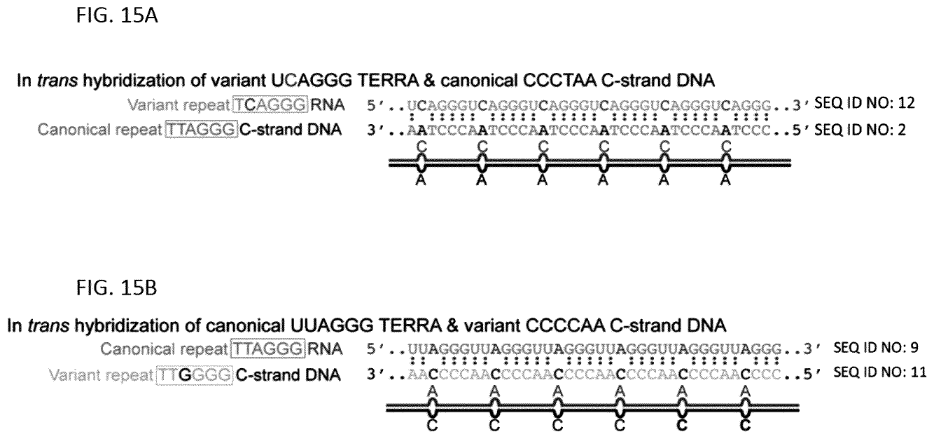

Although ADAR1 does edit A:U base pairs of completely matched dsRNAs, A-C-mismatched base pairs present in naturally occurring dsRNAs such as inverted Alu dsRNAs are, in fact, the favored ADAR1 target sites^{9,50}. Detection of TCAGGG and TTGGGG variant repeats surrounded by TTAGGG canonical repeats within a stretch of telomeric sequence has been reported in HeLa cells³⁸. We hypothesized that RNA:DNA hybrids containing A-C-mismatched base pairs could arise in two ways: first, from slipped binding of telomeric repeat-containing RNAs (TERRAs) derived from a stretch of TCAGGG variant repeats (UCAGGG) to the C-strand of canonical TTAGGG repeats (CCCTAA) (FIG. 6A); second, binding of TERRA RNAs derived from canonical TTAGGG repeats (UUAGGG) to the C-strand of TTGGGG variant repeats (CCCCAA) (FIG. 6B). In particular, TTGGGG variant repeats (both DNA and RNA strands) are prone to the formation of a G-quadruplex structure, which then causes frequent formation of R-loops⁵⁶. G-quadruplex formation of the G-strand TERRAs carrying this particular variant (FIG. 6B, UUGGGG repeats highlighted in orange) is expected to leave its C-strand DNA single stranded, which in turn may cause more frequent slipped hybridization with the canonical repeat G-strand TERRAs (FIG. 6B). Second, there would be an alternative type of pairing, namely in trans formation of RNA:DNA hybrids, which has been previously implicated for telomeric repeat sequences^{25,57}. Thus, in trans hybridization of telomeric repeat RNAs transcribed from one loci either with canonical or variant repeats to C-strand DNAs containing variant or canonical repeats from the other loci, respectively, could also result in telomeric repeat RNA:DNA hybrids containing A-C-mismatched base pairs (FIG. 15A, FIG. 15B). G-strand TERRA RNAs containing variant TCAGGG (UCAGGG) and canonical TTAGGG (UUAGGG) repeat sequences are expected to accumulate in C-A (FIG. 6A and FIG. 15A) and A-C (FIG. 6B and FIG. 15B) mismatch-containing RNA:DNA hybrids, respectively. Similarly, C-strand DNAs containing canonical TTAGGG (CCCTAA) and variant TTGGGG (CCCCAA) repeat sequences are anticipated to be present in C-A (FIG. 6A and FIG. 15A) and A-C (FIG. 6B and FIG. 15B) mismatch-containing RNA:DNA hybrids, respectively. That is exactly what we observed by dot blot analysis of DRIP products using canonical and variant repeat-specific LNA-oligonucleotide probes (FIG. 14C, FIG. 14D): ADAR1 depletion resulted in the accumulation of both RNA and DNA strands of RNA:DNA hybrids consisting of canonical and variant telomere repeats (FIG. 6C, FIG. 6D). These results strongly suggest that ADAR1p110 regulates telomeric RNA:DNA hybrids containing variant repeats and mismatched base pairs.

### ADAR1p110 Edits A-C-Mismatched Base Pairs of Telomeric RNA:DNA Hybrids Formed Between Canonical and Variant Repeats

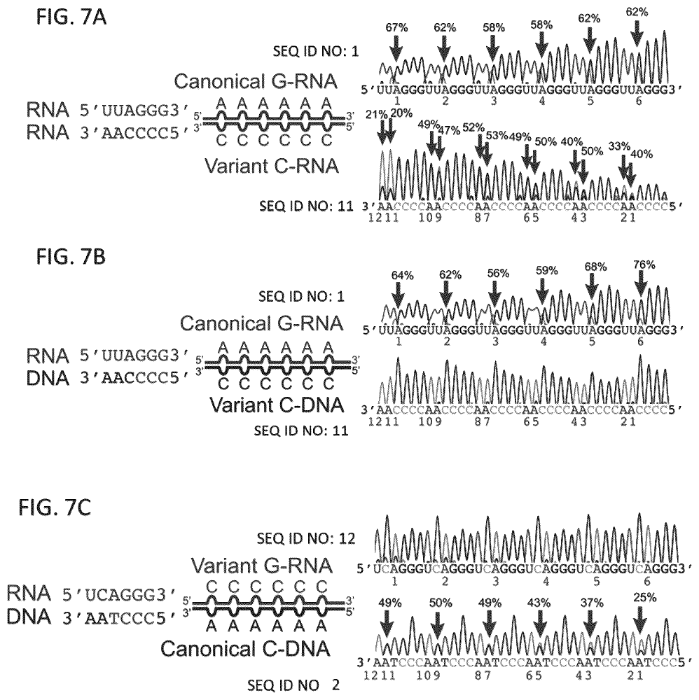

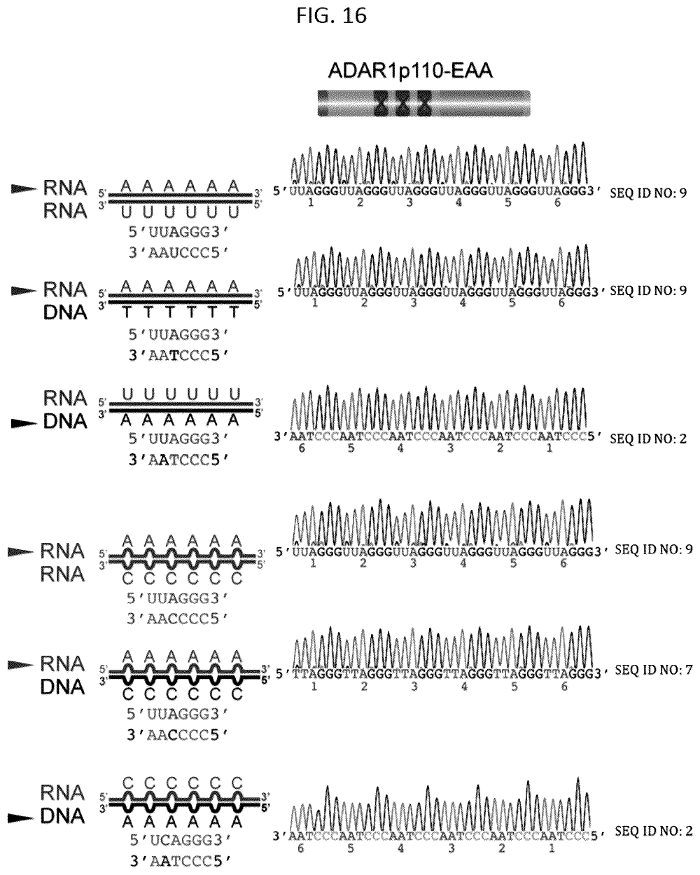

To this end, we prepared additional telomeric repeat duplex substrates containing A-C-mismatched base pairs and tested them for in vitro editing with ADAR1p110. ADAR1p110 again edited very efficiently six adenosine residues of telomeric repeat dsRNA containing A-C mismatches, as expected (FIG. 7A, top). In contrast, ADAR1p110 editing of C-strand adenosines of A-C-mismatched dsRNA was less efficient, perhaps due to the fact that two adenosines are not in the most favored UAG sequence context (FIG. 7A, bottom)⁵⁵. Most importantly, ADAR1p110 edited adenosine residues of both the RNA and the DNA strands of RNA:DNA hybrids, provided that they were at mismatched A-C base pairs (FIG. 7B, top and FIG. 7C, bottom). The level of editing of the opposite strands of these RNA:DNA hybrids, that is, C-strand DNA and G-strand RNA, respectively, was insignificant (FIG. 7B, bottom and FIG. 7C, top). As RNA:DNA hybrid editing activity was lost completely with ADAR1p110-EAA, a dsRNA-binding defective mutant⁵⁸ (FIG. 16), binding of ADAR1p110 to RNA:DNA hybrids must be mediated via its dsRBDs, rather than other domains such as Z-DNA-binding domain (ZO), and is essential for editing of RNA:DNA hybrids. Previous screening has identified ADAR1 as one of many RNA:DNA hybrid-binding proteins, although its biological significance was not addressed⁴⁷.

Editing of A-C Mismatches in Telomeric Repeat RNA:DNA Hybrids Facilitates their Resolution by rNase H2

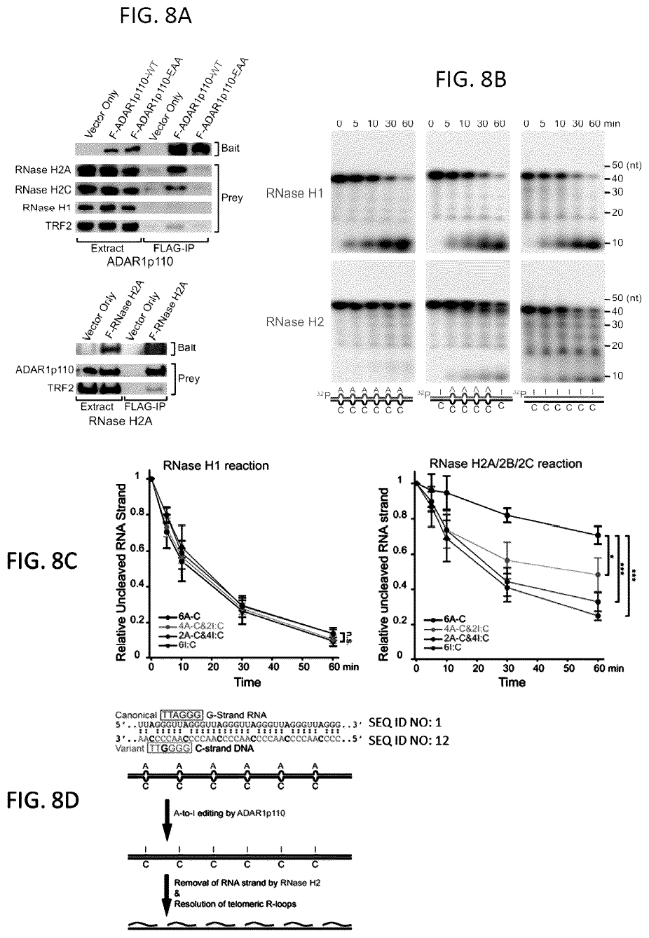

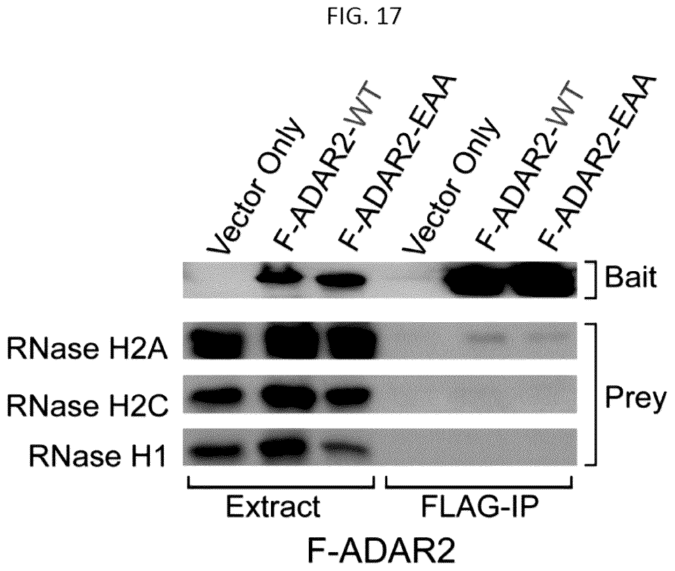

In order to obtain insight into the mechanism by which ADAR1p110-mediated editing of telomeric repeat RNA:DNA hybrids contribute to resolution of telomeric R-loops, we looked for candidate cofactors of ADAR1p110 required for removal of RNA strands from telomeric RNA:DNA hybrids. We conducted immunoprecipitation experiments using HEK293T cells stably expressing FLAG-ADAR1p110-WT. We found that FLAG-ADAR1p110-WT co-immunoprecipitated with rNase H2A and H2C subunits but not with rNase H1 (FIG. 8A, upper panels), revealing the close association of ADAR1p110 with rNase H2 subunits. Similar experiments with FLAG-ADAR2 revealed n59poptosistion of ADAR2 with Rnase H1 or Rnase H2 subunits (FIG. 17). The ADAR1p110 interaction with rNase H2 subunits was lost when FLAG-ADAR1p110-EAA dsRNA-binding defective mutant was used as the bait (FIG. 8A, upper panels). Finally, we conducted a reciprocal pull-down experiment using FLAG-tagged rNase H2A as the bait. FLAG-rNase H2A pulled down endogenous ADAR1p110 (FIG. 8A, lower panels). Furthermore, both FLAG-ADAR1p110-WT and FLAG-rNase H2A pulled down TRF2, a telomere-binding protein and a member of the Shelterin complex^{33,34}. These results together indicate that ADAR1p110 interacts with rNase H2, but not with rNase H1, on the telomeric RNA:DNA hybrids, and possibly collaborates in dissociating telomeric RNA:DNA hybrids containing mismatched A-C base pairs.

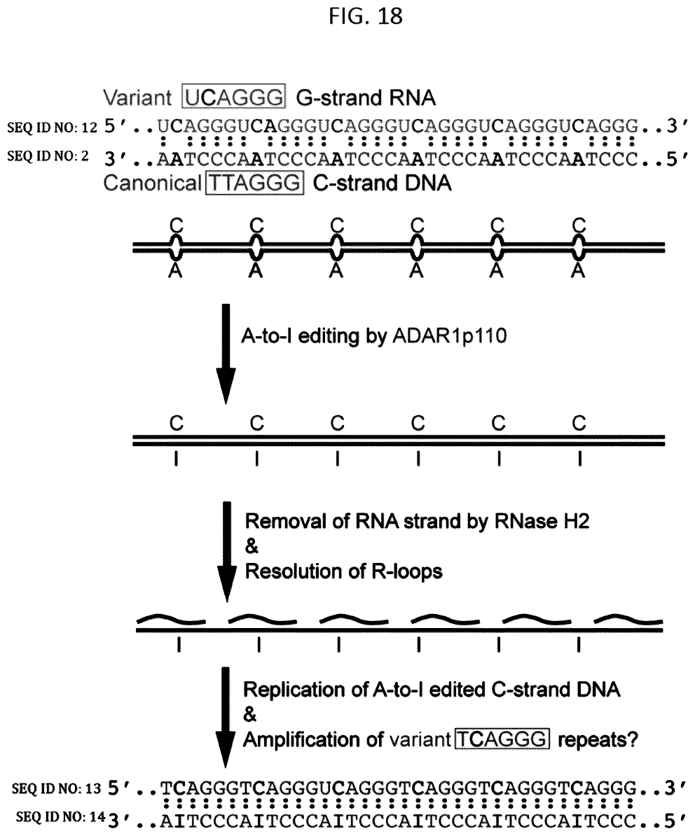

Accordingly, we prepared telomeric repeat RNA:DNA hybrids containing different numbers of A-C-mismatched base pairs as well as I:C-matched RNA:DNA hybrids (mimicking A-to-I-edited hybrids), which were then subjected to in vitro assay using purified human recombinant rNase H1 and rNase H2A/2B/2C complex proteins. To our surprise, rNase H1 digested RNA strands of telomeric repeat RNA:DNA hybrids regardless of the number of A-C-mismatched base pairs (FIG. 8B, upper panels and FIG. 8C, left). In contrast, we found that digestion by rNase H2 is very sensitive to the presence of A-C-mismatched base pairs. The RNA strand of the RNA:DNA hybrids containing six A-C-mismatched base pairs was almost completely resistant to digestion by rNase H2 (FIG. 8B, lower panels and FIG. 8C, right). As A-C-mismatched base pairs are replaced with matched I:C base pairs, the RNA:DNA hybrids became more permissive to rNase H2-mediated digestion (FIG. 8C, right): more efficient digestion for hybrids with more I:C-matched base pairs (FIG. 8D and FIG. 18).

### ADAR1 Depletion Leads to the Accumulation of RNA:DNA Hybrids Only in Non-ALT Cancer Cells

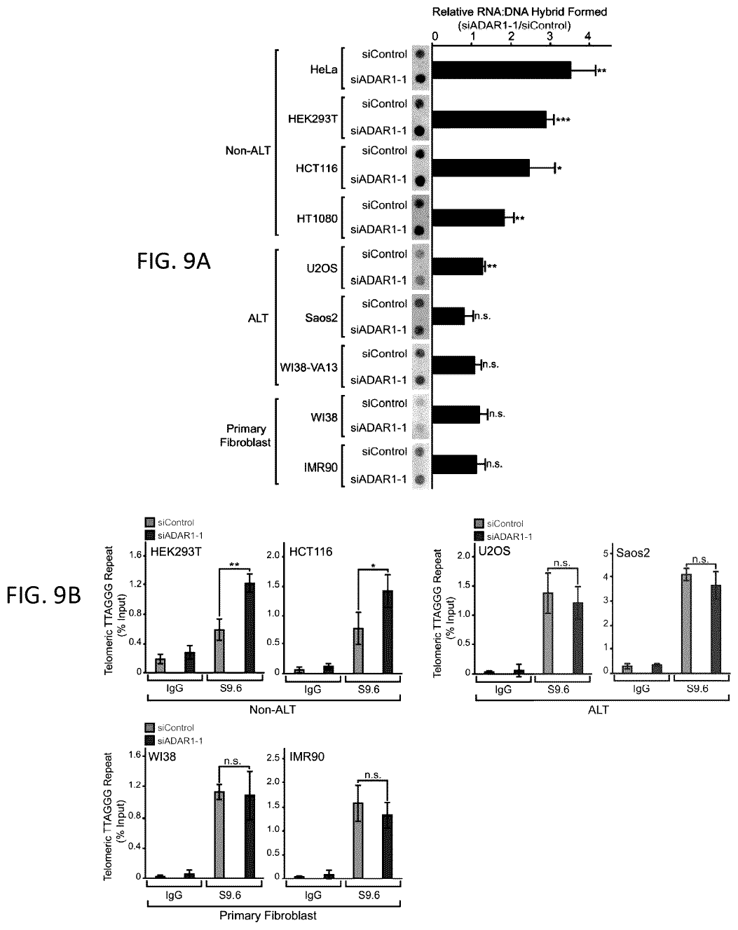

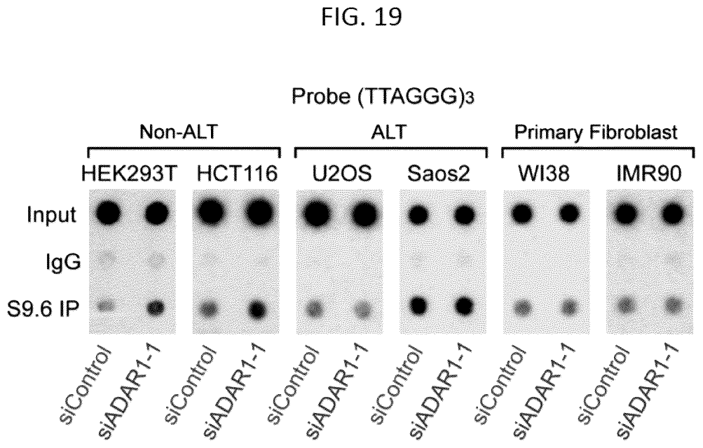

Our FLAG-ADAR1p110 immunoprecipitation experiments revealed the association of ADAR1p110 with rNase H2 subunits, but not with rNase H1 (FIG. 8A). Efficient resolution of telomeric RNA:DNA hybrids by rNase H1 has been reported specifically with ALT activity positive cancer cell lines (ALT cells)⁴⁵. Interestingly, the same study also reported that rNase H1 had no or very little effects on resolution of telomeric RNA:DNA hybrids in non-ALT cell lines⁴⁵, indicating different mechanisms used for regulation of telomeric R-loops in ALT versus non-ALT cancer cells. Accordingly, we investigated the effects of ADAR1 depletion on accumulation of RNA:DNA hybrids in both ALT and non-ALT cell lines as well as primary fibroblast cells. ADAR1 depletion resulted in increased RNA:DNA hybrids in all non-ALT cell lines examined, but had almost no effects in ALT cell lines nor in primary fibroblast cells (FIG. 9A and FIG. 12D). Furthermore, dot blot analysis of DRIP products using a telomeric repeat probe revealed that ADAR1 depletion resulted in the formation of telomeric repeat RNA:DNA hybrids specifically in non-ALT cancer cell lines, for example, HeLa (FIG. 3C), HEK293T, and HCT116 (FIG. 9B and FIG. 19). The reported association of rNase H1 with the strong telomeric repeat R-loops formed only in ALT cells⁴⁵, perhaps highlights the capability of rNase H1 to resolve telomeric RNA:DNA hybrids even in the presence of mismatched base pairs (FIG. 8B, FIG. 8C).

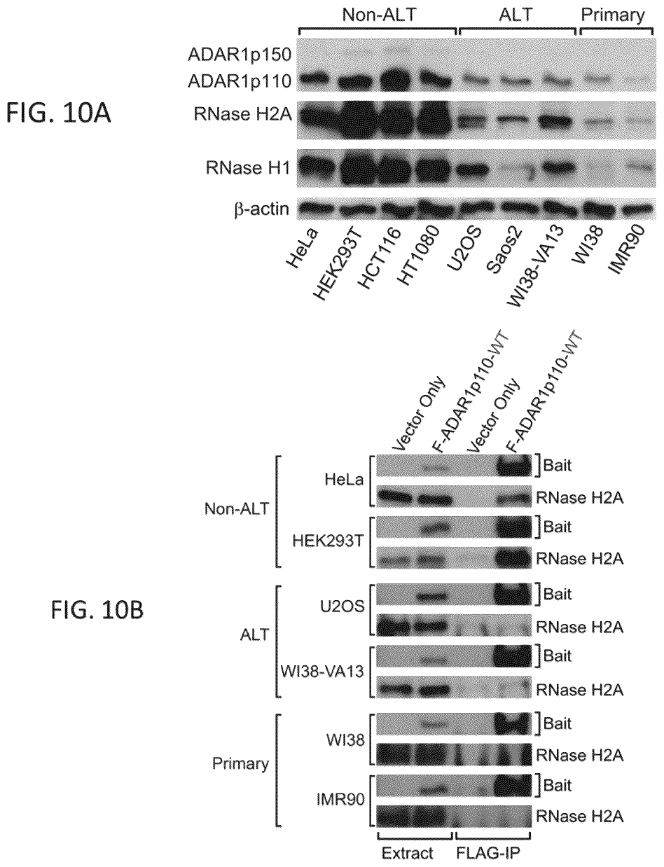

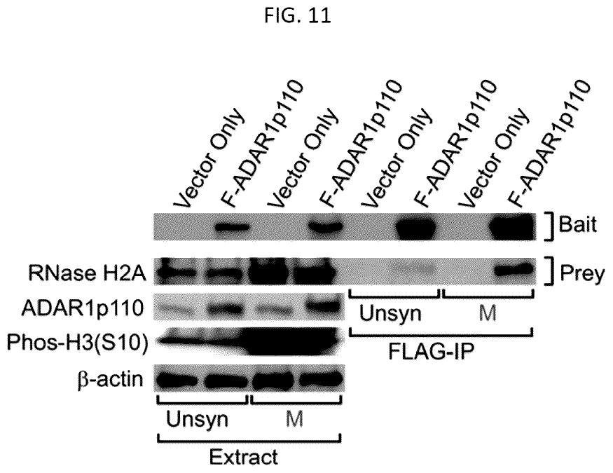

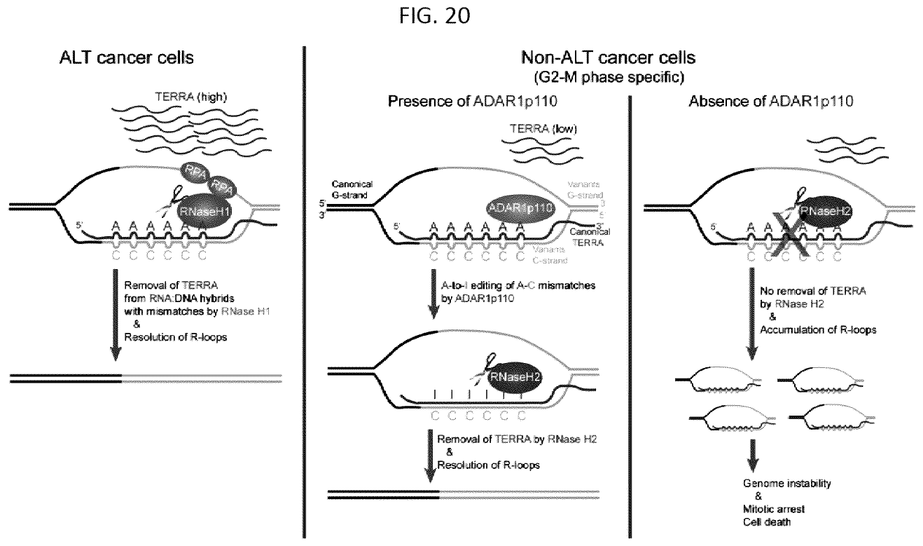

We found that ADAR1p110 and rNase H2A expression levels are much higher in non-ALT cancer cells as compared to those in ALT cancer cells and primary fibroblast cells (FIG. 10A). Furthermore, the interaction of ADAR1p110 with rNase H2A was detected only in non-ALT cancer cells, but not in ALT cancer cells and primary fibroblast cells (FIG. 10B). Interestingly, ADAR1 depletion resulted in the upregulation of M-phase-specific marker genes (FIG. 1D), perhaps indicating an M-phase-specific ADAR1p110 function in suppression of telomeric R-loops. Accordingly, we analyzed the interaction of ADAR1p110 and rNase H2 by F-ADAR1p110 co-immunoprecipitation (co-IP) experiments in cells synchronized to the M phase. First, we noticed that rNase H2A expression levels increased significantly, >2-fold, at the M phase (FIG. 11, compare unsynchronized and M-phase extract lanes), while endogenous ADARp110 levels unchanged (FIG. 11, compare unsynchronized and M-phase extract vector-only lanes). Most importantly, we found that the ADAR1p110-rNase H2A interaction increased >3-fold in M-phase synchronized cells as compared to unsynchronized cells (FIG. 11, FLAG-IP lanes). These results perhaps indicate a special need in non-ALT cancer cells for two-step removal of RNA strands of telomeric RNA:DNA hybrids accumulated specifically around late G2 to M phase: first correction of mismatched base pairs by the nuclear ADAR1p110 and then for degradation of RNA strands by rNase H2 (FIG. 8D and FIG. 18). Taken together, our results indicate a differential role played by rNase H1 and rNase H2 in resolution of R-loops accumulated in ALT and non-ALT cancer cells, respectively, due to their different activity in degrading mismatch-containing telomeric RNA:DNA hybrids (FIG. 20).

### Discussion

Several regulators of telomeric R-loops such as 5′ to 3′ exonuclease, Rat1p⁵⁹, flap endonuclease 1⁶⁰, and rNase H1 and H2^{45,59,61} have been reported. In this study, we identified ADAR1p110 as a major regulator of telomeric R-loops specifically in cancer cells. Variant telomeric repeats such as TCAGGG and TTGGGG repeats are widespread in both ALT and non-ALT cancer cell lines^{38,39} (FIG. 5A, FIG. 5B). Because of these variant repeats, cancer cells encounter a unique problem in solving telomeric RNA:DNA hybrids containing mismatched base pairs. Hybridization of TERRA molecules carrying the G-strand UCAGGG variant sequences to the C-strand DNA carrying the canonical CCCTAA (antisense of TTAGGG) repeats (FIG. 6A and FIG. 15A) or hybridization of TERRA molecules carrying the G-strand canonical UUAGGG sequence to the C-strand DNA carrying the CCCCAA (antisense of TTGGGG) variant sequence (FIG. 6B and FIG. 15B), either by slipped hybridization or in trans hybridization, would result in the formation of telomeric repeat RNA:DNA hybrids containing C-A or A-C mismatches, respectively. The possibility of R-loop formation induced by mismatches between nascent RNA and DNA sequences has been previously pointed out⁶².

We found that ADAR1p110 could edit efficiently A-C-mismatched adenosines of both RNA and DNA strands within telomeric RNA:DNA hybrids and convert them to I:C-matched Watson-Crick base pairs. Interestingly, telomeric variant repeats have been reported to expand during ALT-mediated inter- and/or intra-telomeric recombination in ALT cells, and by a currently unknown mechanism in non-ALT cells^{38,39}. An interesting possibility arises upon A-to-I editing of these mismatched A-C base pairs to I:C-matched base pairs: replication of A-to-I-edited C-strand DNA could generate more variant telomeric TCAGGG repeats (FIG. 18, bottom), perhaps explaining the reported expansion of this variant repeat in cancer cells^{38,39}. Most importantly, we found that A-to-I editing of A-C mismatches within RNA:DNA hybrids is critical for efficient digestion of RNA strands by rNase H2, and consequent resolution of telomeric R-loops (FIG. 8D and FIG. 18), because rNase H2, unlike rNase H1, is incapable of digesting RNA strands of mismatch-containing RNA:DNA hybrids. Our findings on association of ADAR1p110 with rNase H2, but not with rNase H1 in living cells (FIG. 8A), together with elevated expression of rNase H2A subunit (FIG. 10A), especially at the M phase (FIG. 11), further confirms collaboration between ADAR1p110 and rNase H2 in resolving specifically telomeric RNA:DNA hybrids containing A-C mismatches, perhaps at late G2 to M phase, in non-ALT cancer cells (FIG. 20).

We currently do not know the exact reason why a specific requirement of ADAR1p110 for suppression of telomeric R-loops is restricted to non-ALT cells (FIG. 9A, FIG. 9B, and FIG. 20), as widespread presence of telomeric variant repeats has been detected in both ALT and non-ALT cancer cells (FIG. 5A, FIG. 5B). The cell cycle-dependent function of rNase H2, specifically at G2-M phase, in resolution of R-loops, has been reported also in yeast⁶³. In contrast, rNase H1 has been reported to act through the entire cell cycle in yeast⁶³. We confirmed elevated expression of rNase H2 and its interaction with ADAR1p110 specifically at the M phase in non-ALT cancer cells (FIG. 11). Most interestingly, an association of rNase H1 with telomeric RNA:DNA hybrids at strong R-loop-forming loci has been reported only in ALT cancer cells with overly elevated TERRA expression levels⁴⁵ (FIG. 20, left). Association of rNase H1 to telomeric R-loops may also be further facilitated by more frequent binding of RPA32 to the single-stranded G-strand DNA specifically in ALT cells⁶⁴. RPA32 has been shown to enhance the association of rNase H1 and R-loops⁴² (FIG. 20, left). Knockdown of rNase H1 in HeLa cells (non-ALT cells) indeed did not lead to a significant increase of R-loops (FIG. 2D), in agreement with the previous report: rNase H1-dependent resolution of telomeric R-loops occurs only in ALT cells⁴⁵ (FIG. 20, left). TERRA expression levels seem to be another factor that differentiates non-ALT cells from ALT cells. In non-ALT HeLa cells, expression of TERRA is regulated in a cell cycle-dependent manner: lowest in S and early G2, but increasing toward the end of G2 and M phase⁶⁵. Furthermore, TERRA expression levels remain low in non-ALT cells such as HeLa and HEK293T, and thus unable to support strong R-loop-forming loci and recruit rNase H1⁴⁵ (FIG. 20, right). Instead, rNase H2 together with ADAR1p110 is recruited to such not so strong R-loop-forming loci during G2-M phase, and rNase H2 and ADAR1p110 cooperatively resolve telomeric repeat RNA:DNA hybrids containing mismatched base pairs (FIG. 20, right). Clearly, many issues remain to be resolved with regard to why and how two mechanisms are differentially employed for resolution of telomeric R-loops in ALT and non-ALT cancer cells.

Telomere abnormalities such as telomere losses and telomere leading-strand-mediated fusions, most likely caused by unresolved telomeric R-loops, were detected in ADAR1-depleted cells (FIG. 11A-FIG. 11E). Although we have no direct evidence to specify the exact timing, telomeric RNA:DNA hybrids may form and accumulate from the late S phase through M phase in ADAR1-depleted HeLa cells. Unresolved telomeric RNA:DNA hybrids as well as prolonged exposure of single-stranded G-strands most likely lead to double-stranded DNA breaks and eventually to telomere losses and fusions. Apparently, the telomerase activated in non-ALT cells does not mend the shortened telomere ends. Non-homologous end joining and DNA-PKcs participate in telomere end-capping, exclusively at telomeres generated by leading-strand synthesis in non-ALT cancer cells^{66,67}. This end-capping process does not seem to function efficiently in ADAR1-depleted cells. It has been reported that unresolved and persistent R-loops interfere with the DNA double-strand break (DSB) repair mechanism⁶⁸. Although factors involved in DSB repair, such as DNA-PKcs and γH2AX, are activated in ADAR1-depleted cells (FIG. 1D), the DSB repair mechanism may be hindered due to the presence of unresolved RNA:DNA hybrids, which could also contribute to the telomere abnormalities detected. Additional and specific studies will be required to address these issues.

Recent elegant studies by Nicholas Haining and his colleagues²⁰ revealed the possibility that ADAR1 inhibitors could restore MDA5-MAVS-IFN signaling and inflammatory responses in tumors and resurrect their response to therapy utilizing immune checkpoint blockade.

However, our studies presented here suggest another possibility: elimination of ADAR1 and/or suppression of IA-to-I editing activity would lead to the accumulation of telomeric repeat R-loops and consequent genome instability and apoptosis particularly in non-ALT and telomerase-positive cancers, which are, in fact, 70-80% of all types of cancers⁴⁵. We predict that ADAR1 inhibitors would be very effective therapeutics for cancer treatment because they will interfere with two completely different pro-oncogenic ADAR1 functions: suppression of MDA5-MAVS-IFN signaling by the cytoplasmic ADAR1p150 and maintenance of telomere stability in telomerase-reactivated cancer cells by the nuclear ADAR1p110.

### Example 3: Multi-Step Screening Strategy—First Screen

Our results indicate that inhibition of the ADAR1 A-to-I editing activity results in accumulation of telomeric R-loops specifically in telomerase reactivated cancer cells and consequently their apoptotic cell death. Using a permanently transformed HeLa cell line, we confirmed that this reporter system (FIG. 24A) can be used to monitor very specifically the ADAR1-mediated A-to-I RNA editing activity (FIG. 24B); the reporter activity was reduced only in ADAR1 depleted cells (FIG. 24B). We noted that inhibition of ADAR1 editing activity resulted in “slow” induction of apoptosis due to telomere instability and mitotic arrest, which can be measured as reduction in expression of Firefly Luciferase (FFL) (FIG. 24C), specifically in non-ALT cancer cells (FIG. 24D).

A HeLa cell line (telomerase positive) permanently transformed with a dual luciferase reporter system (HeLa-Nluc-edit) as described by Fritzell, K. et al (Sensitive ADAR editing reporter in cancer cells enables high-throughput screening of small molecule libraries. Nucleic acids research 47, e22, doi:10.1093/nar/gky1228 (2019), which is incorporated herein by reference) were seeded at a density of 800 cells per well into 384 white plates (PerkinElmer, CulturPlate384, #6007689) in 20 μl of Opti-MEM without phenol red (Thermo Fisher Scientific #11058021) using a multidrop dispenser (Thermo Fisher Scientific) and incubated overnight at 37-C, 5% CO2. The cells were then treated with test compounds (Maybridge HitFinder Diversity Set library) for 72 h prior to luminescence signal detection. Briefly, 10 μl of Steadylite Plus FFL assay system reagents were added and firefly luminescence signal was measured within 10-20 min after the addition using a multiplate reader at 1 s integration time. Firefly luciferase activity was calculated for each well and results for each plate were normalized to a negative (0.1% DMSO) and positive (Doxorubicin 10 microM) control.

Approximately 1,600 compounds were identified from 14,400, which showed at least a 30% decrease (at least 30% and up to 100% decrease) of FFL activity as compared to the control, which was used as a measure of cell viability/apoptosis. These compounds were further tested in the subsequent screens.

### Example 4: Multi-Step Screening Strategy—Second and Third Screens

HeLa-Nluc-edit cells were seeded at a density of 15,000 cells per well into 384 white plates (PerkinElmer, CulturPlate384, #6007689) in 20 μl of Opti-MEM without phenol red (Thermo Fisher Scientific #11058021) using a multidrop dispenser (Thermo Fisher Scientific) and incubated overnight at 37-C, 5% CO2. The cells were then treated with the 1,600 test compounds identified in the First Screen (Example 3) for 24 h prior to luminescence signal detection.

For the second screen, 10 μl of Steadylite Plus FFL assay system reagents were added and firefly luminescence signal was measured within 10-20 min after the addition using a multiplate reader at 1 s integration time. Firefly luciferase activity was calculated for each well and results for each plate were normalized to a negative (0.1% DMSO) and positive (Doxorubicin 10 microM) control.

For the third screen, 20 μl of ONE-Glo EX reagent (Nano Glo Dual Luciferase assay) was added and luminescence signal was measured within 10-20 min after the addition using a multiplate reader at 1 s integration time. Subsequently, 20 μl NanoDLR Stop &Glo Reagent (Nano Glo Dual Luciferase assay) (Promega) were added to the same wells and luminescence signal was measured with Envision (PerkinElmer) multiplate reader at 0.1 s integration time, at least 10 min after reagent addition. The ratio between Nluc and FFL signal was calculated for each well and results for each plate were normalized to a negative (0.1% DMSO) and positive (Nluc inhibitor 1 10 microM) control.

54 compounds were selected from the 1,600 compounds identified in the first screen. These compounds showed less than 40% decrease of FFL activ^{it}y (2^{nd} selection) & more than 60% inhibition of the reporter activity (Nluc/FFL=A-to-I editing activi^{ty}) (3^{rd} selection).

### Example 5: In Vitro Screen

The 54 compounds identified in the high-throughput screen were further tested in the in vitro editing assay described in Examples 1 and 2. The in vitro editing reaction mixture, containing 5 nM of telomere RNA:RNA duplex substrates and 75 nM of HAT-ADAR1p110-WT, FLAG-ADAR1p110-WT, or HA-ADAR1p110-EAA protein, was incubated at 37° C. for 2 h in in vitro editing buffer I (20 mM HEPES-KOH pH 7.5, 100 mM NaCl, 0.01% NP-40, 5% glycerol, 1 mM DTT) with 10 microM test compound. For editing of RNA:DNA hybrid substrates, in vitro editing buffer II (20 mM HEPES-KOH pH 7.5, 20 mM NaCl, 0.01% NP-40, 5% glycerol, 1 mM DTT) was used. Edited RNA or DNA strands were purified using Dynabeads MyOne Streptavidin C1 (Thermo Fisher Scientific). To remove opposite RNA or DNA strands, Rnase H (NEB) or TuRBO Dnase (Thermo Fisher Scientific) was used, respectively. For sequencing of edited substrates, reverse transcription-PCR was carried out for RNA strands, while PCR was carried out for DNA strands. RT reactions were carried out using SuperScript III Reverse Transcriptase (Thermo Fisher Scientific), and PCR reactions were performed using Platinum Taq DNA polymerase (Thermo Fisher Scientific). PCR products were sequenced using a specific sequencing primer, and the ratio of A and G peaks in the chromatograms were analyzed by CodonCode Aligner (CodonCode Corporation).

The top 9 hits (identified in Table 3) were further tested in a similar in vitro editing assay for all nine compounds using two dsRNA substrates and also one additional DNA:RNA substrate, which resulted in estimation of IC50 values for some of these compounds (Table 4).

TABLE 4
IC50 estimated by in vitro editing assay.
IC50

| Compound | RNA:RNA | RNA:mmRNA | DNA:mmRNA | |
|---|---|---|---|---|
| #1 | 27.0 μM | 33.0 μM | N/A | |
| #6 | N/A | N/A | 30~76 | μM |
| #13 | 3.0 μM | Position 2: 21.0 μM | 30~100 | μM |
| | | Position 5: 8.2 μM | | |
| | | Position 6: 3.8 μM | | |
| #23 | 66.9 μM | Position 6: 66.4 μM | N/A | |
| #24 | N/A | N/A | N/A | |
| #27 | N/A | N/A | 5~17 | μM |
| #37 | Position 2: 68.5 μM | 46 μM | 3~10 | μM |
| | Position 3 & 6: | | | |
| | 3 μM | | | |
| #40 | 57.6 μM | 27.2 μM | N/A | |
| #52 | N/A | N/A | 1.5~3 | μM |

### Example 6: Selective Killing of Cancer Cells but not Normal Fibroblast Cells by ADAR1 Inhibitor Compounds

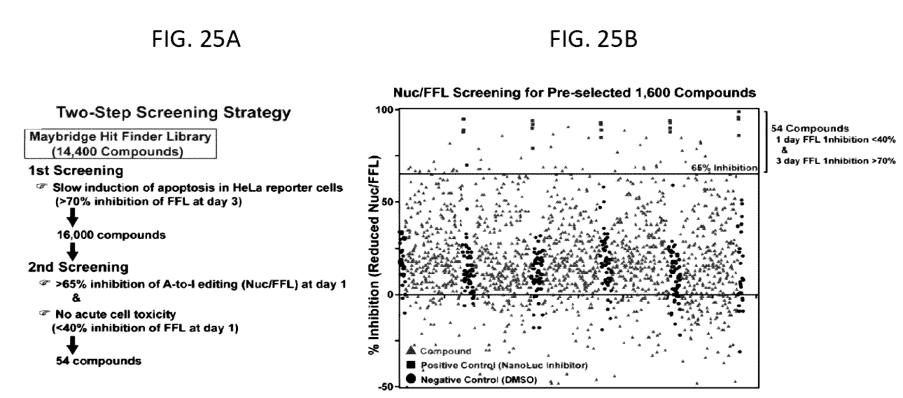

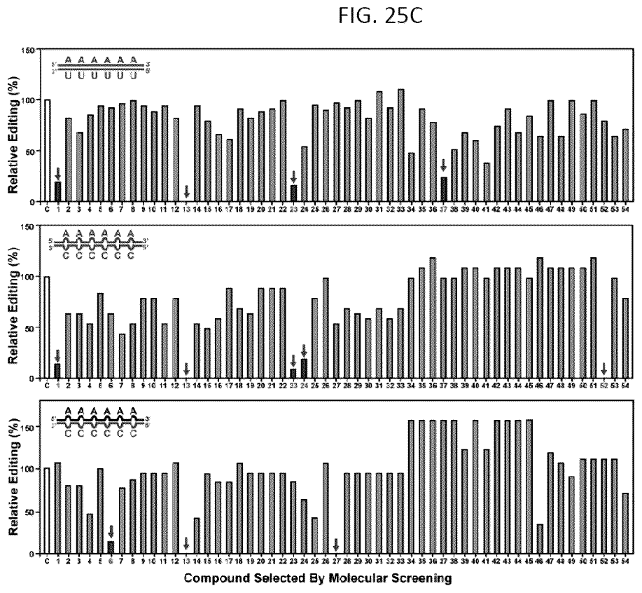

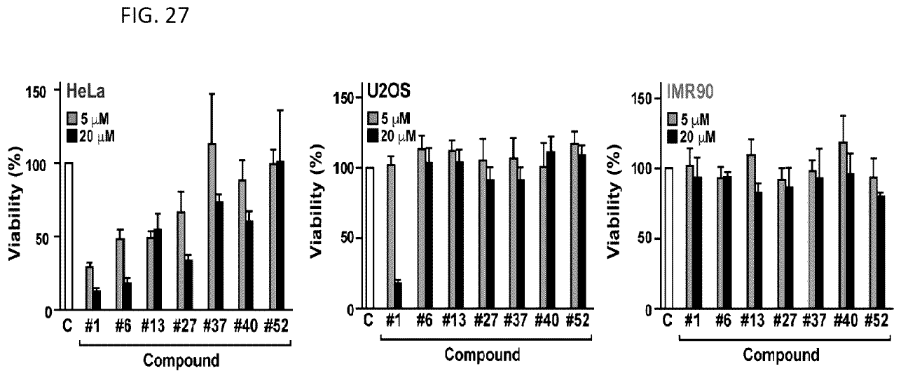

We previously showed that knockdown of ADAR1 induced apoptosis specifically in non-ALT (telomerase reactivated) cancer cells but not in ALT cancer cells or normal fibroblast cells. ADAR1p110 mediated editing of telomeric repeat R-loops is essential for maintenance of telomere stability and consequently continued proliferation of non-ALT cancer cells. Accordingly, effective ADAR1 inhibitors are expected to induce apoptosis specifically in non-ALT cancer cells. We tested select compounds for their effects on viability of various cell lines. We found that certain compounds such as #1, #6, #13, and #27 significantly decreased viability of HeLa (non-ALT) but had very little effects on U2OS (ALT) or MRI90 (normal fibroblast) cells at a concentration of 5 μM (FIG. 27). We plan to modify the chemical structures of certain compounds, e.g., #37, #40, and #52, which may increase in vivo stability and consequently their ADAR1 inhibitory effects in vivo. Our representative analysis conducted with FLAG-ADAR1p110 revealed nine compounds, which indeed exhibited inhibitory effects specifically for ADAR1, but not ADAR2 (data not shown) (FIG. 25C). Interestingly, we noted some substrate specific inhibition of some compounds: for instance, compound 52 inhibited A-C mismatched dsRNA but not A:U matched dsRNA (FIG. 25C).

### Example 7: Further Characterization of Compound #13

Eight of the “hit” compounds were tested for in vitro editing inhibition dose dependency, selective cancer cell killing, and their potency for induction of DNA damage, M phase arrest, and apoptosis. Compound #13 (3-chloro-N-(4-cyanophenyl)-1-benzothiophene-2-carboxamide) was selected for further studies.

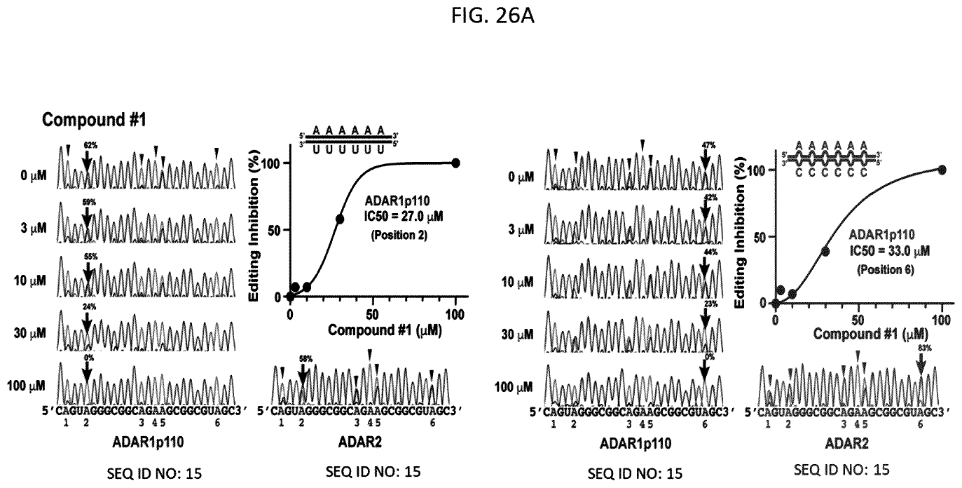

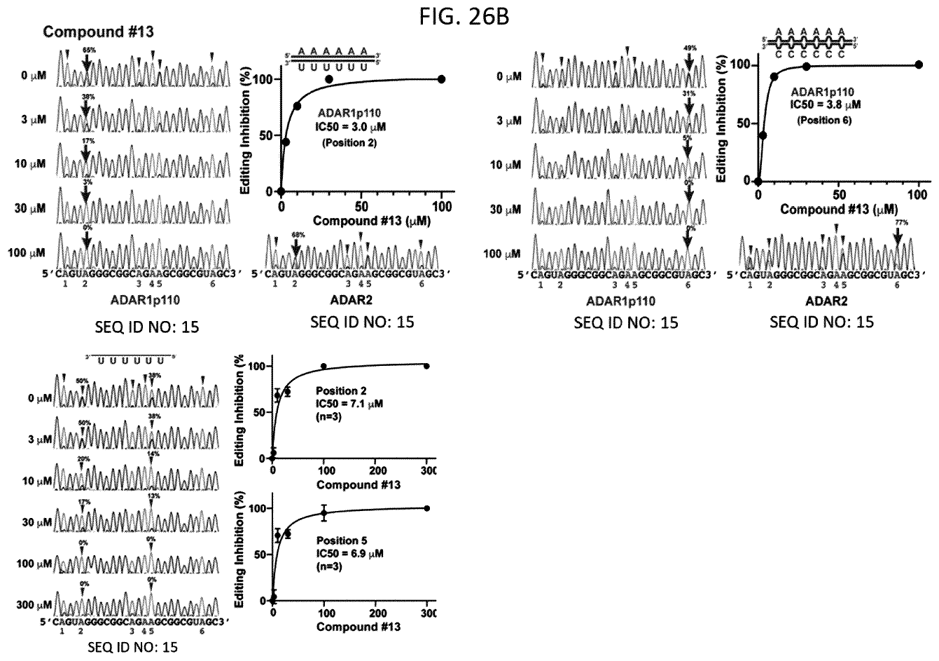

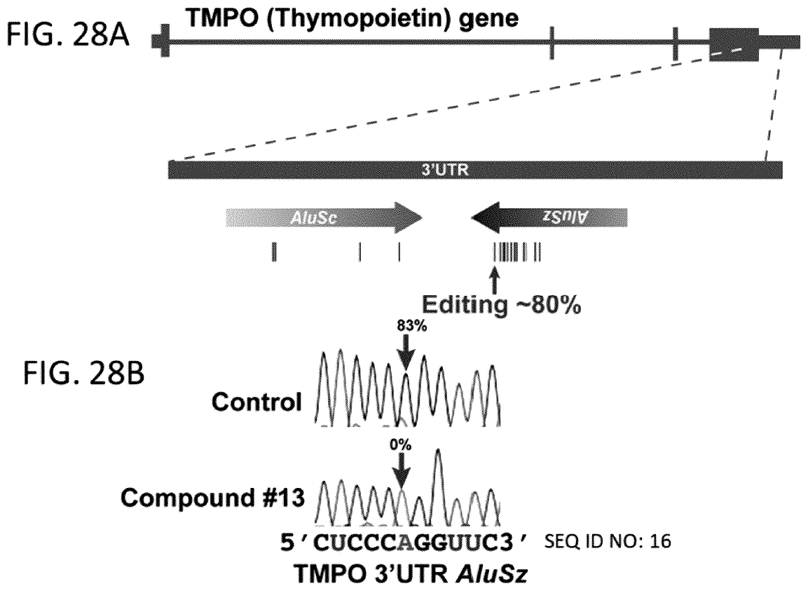

We conducted in vitro editing assay at varying concentrations of Compound #13 with dsRNAs (matched, mismatched) and DNA:RNA hybrids, resulting in determination of IC50 values for ADAR1p110 and ADAR1p150 (not shown) in 7-40 μM ranges: examples of such experiments done for dsRNA are shown (FIG. 8). Compound #13 is very hydrophobic: LC-MS/MS analysis revealed that it is only 0.59 μM water soluble when tested at 100 μM in 1% DMSO. Thus effective (water soluble) concentrations of IC50 values of Compound #13 are likely to be much less than ones determined (FIGS. 26A and 26B). In vivo ADAR1 inhibitory effects in HeLa cells were examined by cDNA sequencing of RT-PCR products of known ADAR1 target RNAs. One example of analysis of such ADAR1 target sites located within the 3′UTR Alu dsRNA of TMPO mRNAs is shown (FIGS. 28A and 28B). This particular ADAR1 target site is highly edited in HeLa cells, more than 80%. Compound #13 inhibited this ADAR1 target site editing down to 0% (FIGS. 28A and 28B).

### Example 8: Analysis of ADAR1 Inhibitor Compounds for their Selective Killing of Telomerase Reactivated Cancer Cells

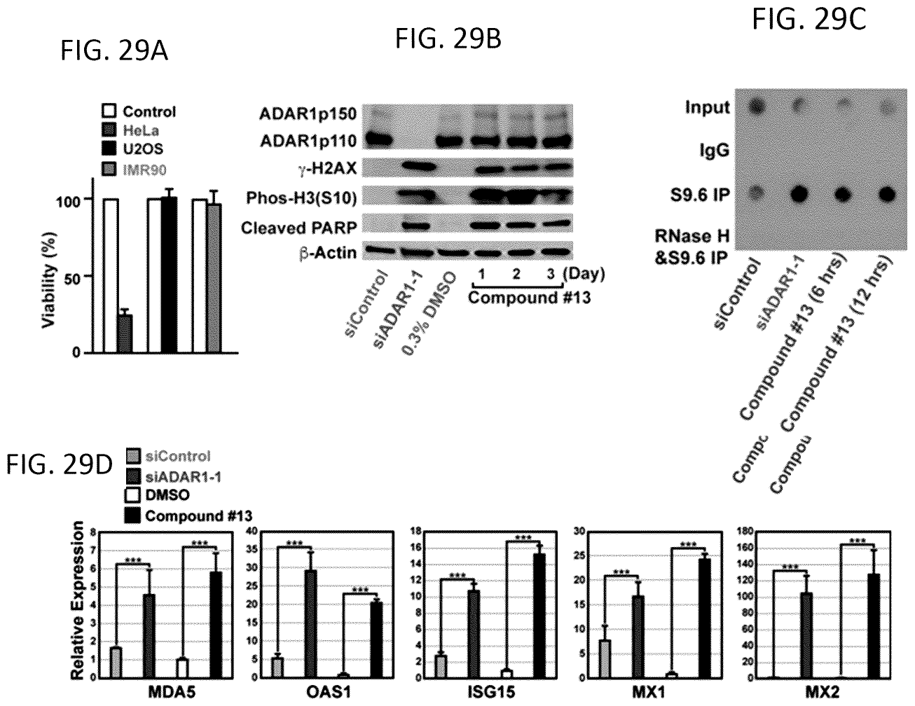

We previously found that acute knockdown of ADAR1 resulted in accumulation of R-loops and extensive DNA damage at telomeres and consequently M phase arrest and apoptosis of non-ALT (or telomerase reactivated) cancer cells (FIG. 5D) (3). We will investigate new ADAR1 inhibitors for their selective killing of telomerase reactivated cancer cells and conduct western blotting analysis to confirm activation of DNA damage, M phase arrest, and induction of apoptosis. We carried out studies for Compound #13. We confirmed that Compound #13 indeed killed selectively non-ALT HeLa cells but not ALT U2OS cells or IMR90 normal fibroblast cells (FIG. 29A) and induced DNA damage, M phase arrest, and apoptosis (FIG. 10B), and increased formation of R-loops (RNA:DNA hybrids) (FIG. 29C).

### Example 9: Analysis of ADAR1 Inhibitor Compounds for their Potency to Activate IFN Signaling in Cancer Cells

Inhibition of ADAR1 mediated A-to-I editing of 3′UTR Alu dsRNAs activates the dsRNA sensing mechanism and induces IFNs (FIG. 2A). This activation of IFN signaling is required to overcome the tumor resistance to PD-1 based immunotherapy (FIG. 2B) (2), one of our motivations to identify ADAR1 inhibitors. Our preliminary studies conducted with Compound #13 revealed that this ADAR1 inhibitor upregulated expression of known ISGs, MDA5, OAS1, ISG15, MX1, and MX2 (FIG. 29D).

### Example 10: ADAR1 Inhibition Mechanisms

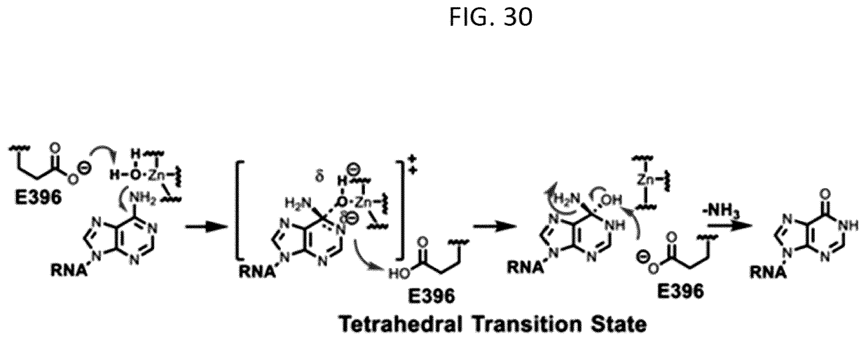

ADAR enzymes catalyze the deamination at C6 of adenosine in dsRNA to generate inosine at the corresponding position (15, 16). ADARs use a zinc-activated water molecule and a glutamic acid residue (E396 in ADAR2 and E912 in ADAR1) for proton transfer (51, 56). The metal bound hydroxide acts as a nucleophile to attack the C6 position of the adenosine ring to form a tetrahedral intermediate. The collapse of this intermediate results in formation of the inosine product (FIG. 30). Hydration of 8-azanebularine has been used as a mimic of the proposed tetrahedral transition state for the ADAR RNA-editing reaction to provide X-ray structures of the complex with ADAR2 (56). This transition state mimetic with 8-azanebularine containing RNA provides structural details of the RNA-ADAR complex to facilitate probe and inhibitor design (56).

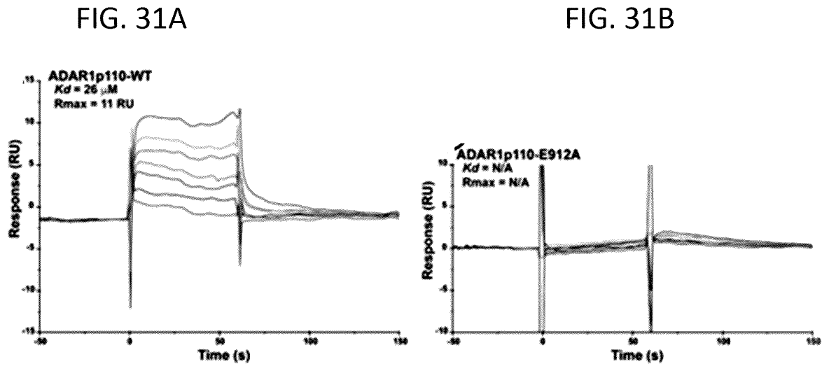

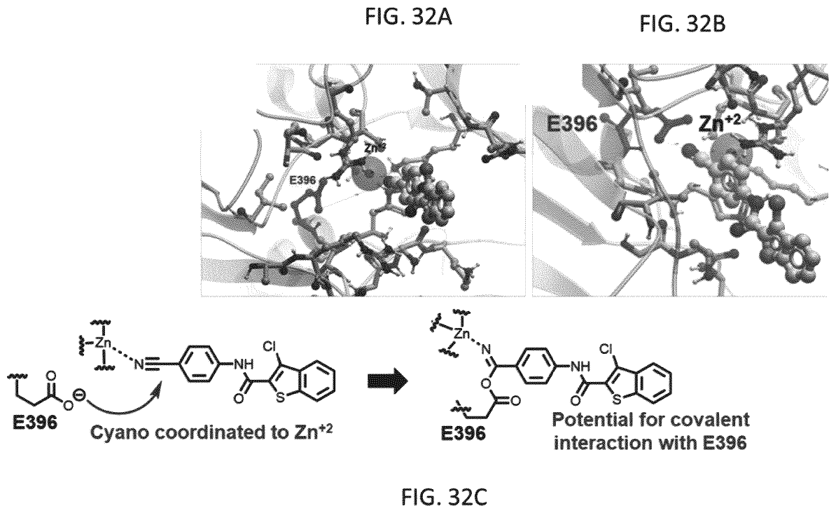

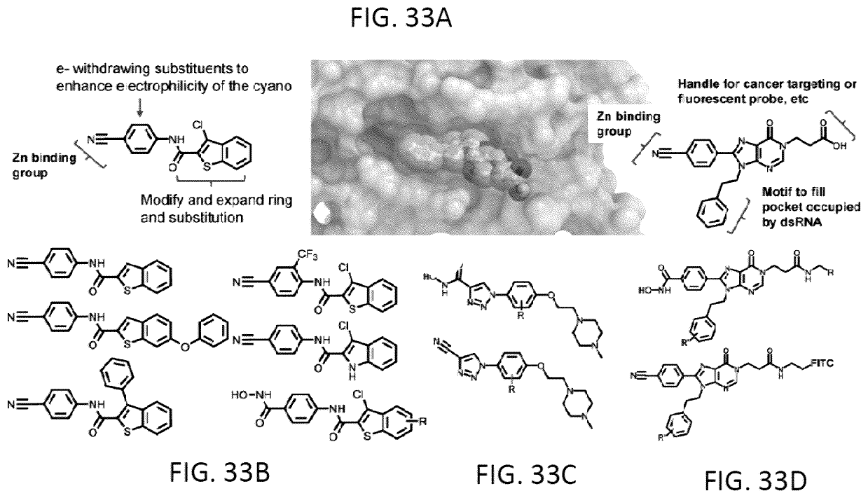

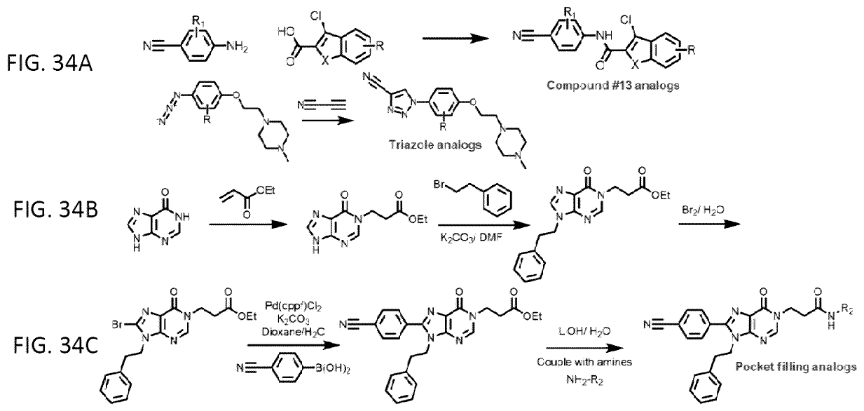

We synthesized 2 additional analogs of Compound #13 for preliminary structure activity relationships (SAR), namely, #13-Analog 1 and #13-Analog 2 (Table 3). Compound #13-Analog 1 lacks the 4-cyano moiety and #13-Analog 2 shifts the cyano to the 2-position of the phenyl ring. Loss of the 4-cyano (#13-Analog 1) resulted in complete loss of inhibitory potency of ADAR1 p110 editing activity, whereas #13-Analog-2 was weaker but retained A-to-I editing inhibitory activity (data not shown). We hypothesized that the cyano-phenyl moiety will coordinate with the zinc atom in the active site when bound to ADAR1. We evaluated the ability of Compound #13 to bind to ADAR1 using surface plasmon resonance (SPR) on the Biacore T200. Compound #13 selectively bound to ADAR1p110-WT (Kd ˜26 μM) (FIG. 31A) relative to a non-related negative control protein GAPDH (not shown). We then evaluated binding to the deamination defective mutant ADAR1p110-E912A, where E912 is the active site glutamic acid (equivalent to E396 in ADAR2) and found that Compound #13 did not bind to this mutant (FIG. 31B). We modeled Compound #13 into the ADAR2 active site (56). Although Compound #13 does not occupy areas in the pocket filled by dsRNA, it can easily fit into the active site and coordinate with the zinc atom (FIG. 32A, 32B). The modeling suggests that the zinc-activated-cyano group may be able to react with the E396 to form an imidate-like structure or hydrolyze to a carboxamide due to reaction with water (FIG. 32C).

Taking all the data together indicates that the cyanophenyl moiety in Compound #13 is important for binding to wild type ADAR1 where loss of the cyano (#13-Analog 1) results in an inactive compound. It also suggests that the cyano may react with the active site glutamic acid to form a covalent adduct to enhance binding affinity, since Compound #13 did not bind to mutant E912A ADAR1 (FIG. 31B).

### Example 11: Mouse Tumor Models

Our studies indicate that inhibition of ADAR1 editing activity selectively killa telomerase reactivated cancer cells. Furthermore, inhibition of ADAR1 editing activity induces type I IFN signaling, which overcomes resistance to ICB (2). ADAR1 inhibitors will serve as effective cancer therapeutics, selectively killing telomerase reactivated cancers, and restoring tumor response to PD-1 based immunotherapy. We will test the efficacy of ADAR1 inhibitors in vivo in mouse tumor model systems. We will use two well established cancer models, one melanoma and the other ovarian cancer.

In vivo evaluation of ADAR1 inhibitors in mouse melanoma models.

The efficacy of ADAR1 inhibitors alone or in combination with anti-PD1 Ab will be assessed using murine melanoma cell lines (telomerase reactivated) as previously described (Wang J, UV-induced somatic mutations elicit a functional T cell response in the YUMMER1.7 mouse melanoma model. Pigment Cell Melanoma Res. 2017; 30(4):428-35. Epub 2017/04/06. PubMed PMID: 28379630; PMCID: PMC5820096.). Mouse melanoma models have been used for investigation of ADAR1 gene knockout effects on ICB (Ishizuka J J, Loss of ADAR1 in tumours overcomes resistance to immune checkpoint blockade. Nature. 2019; 565(7737):43-8. Epub 2018/12/19. PubMed PMID: 30559380.). We will perform these studies using syngeneic murine B16 melanoma cell line (Ishizuka J J, Loss of ADAR1 in tumours overcomes resistance to immune checkpoint blockade. Nature. 2019; 565(7737):43-8. Epub 2018/12/19. PubMed PMID: 30559380) and also YUMM 1.7 cell line derived from BrafV600E/wt Pten−/− Cdkn2−/− mice (Meeth K. The YUMM lines: a series of congenic mouse melanoma cell lines with defined genetic alterations. Pigment Cell Melanoma Res. 2016; 29(5):590-7. Epub 2016/06/12. PubMed PMID: 27287723; PMCID: PMC5331933.). YUMM 1.7 cell line will complement the studies with B16 cell line. YUMM 1.7 cells possess a low number of somatic genetic changes and, in general, are minimally immunogenic, offering a suitable model to study sensitization to ICB.

To study the effect of ADAR1 inhibitors in the context of an intact immune system, tumor cells will be implanted subcutaneously into syngeneic C57BL/6 mice (both male and female). We will first conduct a pilot study to determine the maximum tolerated (MTD) dose of the ADAR1 inhibitor (ADAR1i). We will then assess the effect of a low dose, intermediate dose, and MTD of the selected ADAR1i inhibiting tumor growth. To do this, tumors will be implanted subcutaneously, once tumors become palpable (50-100 mm3), mice will be randomized into different treatment groups (vehicle, low dose, intermediate and MTD). Mice will be treated daily via intraperitoneally (IP) for 2-3 weeks and tumor growth and animal survival will be determined. For combination studies, the effective dose of ADAR1i and lower dose will be tested. In these studies, mice bearing established tumors will be randomized into five different treatment groups: i) vehicle+control IgG, ii) ADAR1i+control IgG, iii) vehicle+anti-PD-1 Ab, vi) ADAR1i (low dose)+anti-PD-1 Ab, v) ADAR1i (effective dose)+anti-PD-1 Ab. Combination parameters to be established include optimal doses, schedule, and combination regime. In the first set of experiments, the top candidate ADAR1i (previous section) will be given first to prime the tumor for anti-PD1 mediated immune response. Unless toxicity issues prevent it, mice will be dosed with the ADAR1 inhibitor compound throughout the experiment. The anti-PD-1 antibody (low endotoxin, azide-free, 0.1 EU/μg, purified rat anti-mouse PD-1 antibody, clone RMP1-14, BioLegend) or control rat IgG antibody will be injected intraperitoneally at a concentration of 10 mg/kg three times per week for four weeks. We will monitor the tumor growth by ultrasound imaging or digital calipers twice a week. Animals will be euthanized on day 21 or when tumors reach 2.0 cm3; endpoints will be tumor volume, and survival. The percent Tumor Growth Inhibition (% TGI) will be calculated and reported for each of the treatment groups (T) versus control (C) using initial (i) and final (f) tumor measurements by the following formula: % TGI=[1−(Tf−Ti/Cf−Ci)]×100.

Mice will be bled early-on-treatment (EOT), at the time of tumor response (TTR) and at the end of study (EOS). Markers of immune response (Li H, van der Merwe P A, Sivakumar S. Biomarkers of response to PD-1 pathway blockade. Br J Cancer. 2022; 126(12):1663-75. Epub 2022/03/02. PubMed PMID: 35228677) and levels of circulating tumor cells (CTC) will be assessed. Tumors will be digested using a Tumor Dissociation Kit and gentle MACS Dissociator (Milentyi Biotec). The immune cell infiltration will be assessed by flow cytometry (CD45 positivity). T cell subsets will be defined based on CD3, CD8, and CD4 positivity. If strong responses are observed upon treatment, half of the treated mice will be euthanized and the other half will be continuously monitored to determine progression free survival. CTCs (CD45−) will be determined by flow cytometry as previously described (Reyes-Uribe P, Exploiting TERT dependency as a therapeutic strategy for NRAS-mutant melanoma. Oncogene. 2018; 37(30):4058-72. Epub 2018/04/27. PubMed PMID: 29695835; PMCID: PMC6062502). Cytokine expression in sorted cells will be evaluated by qRT-PCR and intracellular cytokine staining. We will monitor activation of IFN signaling and ISGs in tumors and host mice by RNA-seq analysis (FIG. 10D). Another tumor aliquot will be digested, and myeloid and lymphoid fractions of infiltrating immune cells will be collected using FACS.

If we observe a positive correlation between changes in certain immune modulating cell populations and tumor suppressive effects, we will perform loss of function studies to determine whether these changes account for or contribute to the observed tumor suppressive effects in the combination treatment. For example, if we observe a correlation with an increase in CD8+ cytotoxic T cells in the combination treatment group compared with either treatment alone, we will deplete CD8+ T cells using an anti-CD8 antibody as we previously published in the combination treatment (Zhu H, BET Bromodomain Inhibition Promotes Anti-tumor Immunity by Suppressing PD-L1 Expression. Cell reports. 2016; 16(11):2829-37. Epub 2016/09/15. PubMed PMID: 27626654; PMCID: PMC5177024.). This will allow us to determine whether the depletion of CD8+ T cells is sufficient to block the observed synergy in the combination treatment group. These results will inform us whether ADAR1 inhibitor-induced changes in the immune modulating cells such as CD8+ T cells in the tumor microenvironment contribute to or account for the observed tumor suppressive effects in the combination treatment group.

In vivo evaluation of ADAR1 inhibitors in the mouse ovarian cancer model.

Similar to melanoma studies described above, we will test ADAR1 inhibitors also in mouse ovarian cancer model. We will conduct these studies using the murine fallopian tube derived ovarian cancer cell line BPPNM (telomerase reactivated) (Iyer S, Genetically Defined Syngeneic Mouse Models of Ovarian Cancer as Tools for the Discovery of Combination Immunotherapy. Cancer Discov. 2021; 11(2):384-407. Epub 2020/11/08. PubMed PMID: 33158843; PMCID: PMC8344888). Notably, BPPNM tumors are typically unresponsive to single-agent ICB treatment and ICBs are effective only when immune-stimulating alterations occur to the tumor microenvironment such as IFN signaling induced by adjuvant treatments (Iyer S, Genetically Defined Syngeneic Mouse Models of Ovarian Cancer as Tools for the Discovery of Combination Immunotherapy. Cancer Discov. 2021; 11(2):384-407. Epub 2020/11/08. PubMed PMID: 33158843; PMCID: PMC8344888). Thus, this model is ideally suited for our study because ADAR1 inhibitors induce IFN signaling. In addition, we will validate our findings in additional syngeneic ovarian cancer models using cell lines such as ID8 and UPK10 previously published (Lin J, Targeting the IRE1alpha/XBP1s pathway suppresses CARMI-expressing ovarian cancer. Nature communications. 2021; 12(1):5321. Epub 2021/09/09. PubMed PMID: 34493732; PMCID: PMC8423755; Lin J, The SETDB1-TRIM28 Complex Suppresses Antitumor Immunity. Cancer Immunol Res. 2021; 9(12):1413-24. Epub 2021/12/02. PubMed PMID: 34848497; PMCID: PMC8647838; Zhao B, Topoisomerase 1 cleavage complex enables pattern recognition and inflammation during senescence. Nature communications. 2020; 11(1):908. Epub 2020/02/23. PubMed PMID: 32075966; PMCID: PMC7031389.). According to the experimental design already described above for melanoma studies, ovarian cancer cells will be injected into bursa sac that covers the ovary in female C57BL6 mice to generate the syngeneic ovarian cancer model. Tumor size will be monitored by ultrasound imaging. In addition to all check points described in the previous section, we will monitor the effects of these treatments on the production of ascites in mice. The efficacy of ADAR1 inhibitors alone or combining with anti-PD1 Ab for suppression of tumor growth will be assessed as previously described (Wu S, Targeting glutamine dependence through GLS1 inhibition suppresses ARID1A-inactivated clear cell ovarian carcinoma. Nat Cancer. 2021; 2(2):189-200. Epub 2021/06/05. PubMed PMID: 34085048; PMCID: PMC8168620; Zhu H, BET Bromodomain Inhibition Promotes Anti-tumor Immunity by Suppressing PD-L1 Expression. Cell reports. 2016; 16(11):2829-37. Epub 2016/09/15. PubMed PMID: 27626654; PMCID: PMC5177024.).

### REFERENCES

1. Eisenberg E, Levanon E Y. A-to-I RNA ed-ting—immune protector and transcriptome diversifier. Nat. Rev. 2018; 19:473-490.
2. Nishikura K. A-to-I editing of coding and non-coding RNAs by ADARs. Nat. Rev. Mol. Cell Biol. 2016; 17:83-96.
3. Walkley C R, Li J B. Rewriting the transcriptome: adenosine-to-inosine RNA editing by ADARs. Genome Biol. 2017; 18:205.
4. Mannion N, Arieti F, Gallo A, Keegan L P, O'Connell M A. New insights into the biological role of mammalian ADARs; the RNA editing proteins. Biomolecules. 2015; 5:2338-2362.
5. Samuel C E. Adenosine deaminase acting on RNA (ADAR1), a suppressor of double-stranded RNA-triggered innate immune responses. J. Biol. Chem. 2019; 294:1710-1720.
6. Lai F, Drakas R, Nishikura K. Mutagenic analysis of double-stranded RNA adenosine deaminase, a candidate enzyme for RNA editing of glutamate-gated ion channel transcripts. J. Biol. Chem. 1995; 270:17098-17105.
7. Macbeth M R, et al. Inositol hexakisphosphate is bound in the ADAR2 core and required for RNA editing. Science. 2005; 309:1534-1539.
8. Porath H T, Carmi S, Levanon E Y. A genome-wide map of hyper-edited RNA reveals numerous new sites. Nat. Commun. 2014; 5:4726.
9. Tan M H, et al. Dynamic landscape and regulation of RNA editing in mammals. Nature. 2017; 550:249-254.
10. Bazak L, et al. A-to-I RNA editing occurs at over a hundred million genomic sites, located in a majority of human genes. Genome Res. 2014; 24:365-376.
11. Sakurai M, et al. A biochemical landscape of A-to-I RNA editing in the human brain transcriptome. Genome Res. 2014; 24:522-534.
12. Patterson J B, Thomis D C, Hans S L, Samuel C E. Mechanism of interferon action: double-stranded RNA-specific adenosine deaminase from human cells is inducible by alpha and gamma interferons. Virology. 1995; 210:508-511.
13. Patterson J B, Samuel C E. Expression and regulation by interferon of a double-stranded-RNA-specific adenosine deaminase from human cells: evidence for two forms of the deaminase. Mol. Cell. Biol. 1995; 15:5376-5388.
14. Liddicoat B J, et al. RNA editing by ADAR1 prevents MDA5 sensing of endogenous dsRNA as nonself. Science. 2015; 349:1115-1120.
15. Mannion N M, et al. The RNA-editing enzyme ADAR1 controls innate immune responses to RNA. Cell Rep. 2014; 9:1482-1494.
16. Pestal K, et al. Isoforms of RNA-editing enzyme ADAR1 independently control nucleic acid sensor MDA5-driven autoimmunity and multi-organ development. Immunity. 2015; 43:933-944.
17. Hartner J C, et al. Liver disintegration in the mouse embryo caused by deficiency in the RNA-editing enzyme ADAR1. J Biol. Chem. 2004; 279:4894-4902.
18. Wang Q, et al. Stress-induced apoptosis associated with null mutation of ADAR1 RNA editing deaminase gene. J Biol. Chem. 2004; 279:4952-4961.
19. Rice G I, et al. Mutations in ADAR1 cause Aicardi-Goutieres syndrome associated with a type I interferon signature. Nat. Genet. 2012; 44:1243-1248.
20. Ishizuka J J, et al. Loss of ADAR1 in tumours overcomes resistance to immune checkpoint blockade. Nature. 2019; 565:43-48.
21. Han L, et al. The genomic landscape and clinical relevance of A-to-I RNA editing in human cancers. Cancer Cell. 2015; 28:515-528.
22. Paz-Yaacov N, et al. Elevated RNA editing activity is a major contributor to transcriptomic diversity in tumors. Cell Rep. 2015; 13:267-276.
23. Roth S H, Levanon E Y, Eisenberg E. Genome-wide quantification of ADAR adenosine-to-inosine RNA editing activity. Nat. Methods. 2019; 16:1131-1138.
24. Niehrs C, Luke B. Regulatory R-loops as facilitators of gene expression and genome stability. Nat. Rev. Mol. Cell Biol. 2020
25. Santos-Pereira J M, Aguilera A. R loops: new modulators of genome dynamics and function. Nat. Rev. 2015; 16:583-597.
26. Lima W F, et al. Viable RnaseH1 knockout mice show RnaseH1 is essential for R loop processing, mitochondrial and liver function. Nucleic Acids Res. 2016; 44:5299-5312.
27. Chon H, et al. Rnase H2 roles in genome integrity revealed by unlinking its activities. Nucleic Acids Res. 2013; 41:3130-3143.
28. Cornelio D A, Sedam H N, Ferrarezi J A, Sampaio N M, Argueso J L. Both R-loop removal and ribonucleotide excision repair activities of Rnase H2 contribute substantially to chromosome stability. DNA Rep. 2017; 52:110-114.
29. Chakraborty P, Grosse F. Human DHX9 helicase preferentially unwinds RNA-containing displacement loops (R-loops) and G-quadruplexes. DNA Rep. 2011; 10:654-665.
30. Skourti-Stathaki K, Proudfoot N J, Gromak N. Human senataxin resolves RNA/DNA hybrids formed at transcriptional pause sites to promote Xm2-dependent termination. Mol. Cell. 2011; 42:794-805.
31. Garcia-Muse T, Aguilera A. R loops: from physiological to pathological roles. Cell. 2019; 179:604-618.
32. Groh M, Gromak N. Out of balance: R-loops in human disease. PloS Genet. 2014; 10:e1004630.
33. Blasco M A. Telomeres and human disease: ageing, cancer and beyond. Nat. Rev. 2005; 6:611-622.
34. de Lange T. Shelterin-mediated telomere protection. Annu. Rev. Genet. 2018; 52:223-247.
35. Lee Y W, Arora R, Wischnewski H, Azzalin C M. TRF1 participates in chromosome end protection by averting TRF2-dependent telomeric R loops. Nat. Struct. Mol. Biol. 2018; 25:147-153.
36. Greenberg R A. Telomeres, crisis and cancer. Curr. Mol. Med. 2005; 5:213-218.
37. Azzalin C M, Lingner J. Telomeres: the silence is broken. Cell Cycle. 2008; 7:1161-1165.
38. Conomos D, et al. Variant repeats are interspersed throughout the telomeres and recruit nuclear receptors in ALT cells. J Cell Biol. 2012; 199:893-906.
39. Lee M, et al. Telomere extension by telomerase and ALT generates variant repeats by mechanistically distinct processes. Nucleic Acids Res. 2014; 42:1733-1746.
40. Sieverling L, et al. Genomic footprints of activated telomere maintenance mechanisms in cancer. Nat. Commun. 2020; 11:733.
41. Higuchi M, et al. Point mutation in an AMPA receptor gene rescues lethality in mice deficient in the RNA-editing enzyme ADAR2. Nature. 2000; 406:78-81.
42. Nguyen H D, et al. Functions of replication protein A as a sensor of R loops and a regulator of RnaseH1. Mol. Cell. 2017; 65:832-847 e834.
43. Hamperl S, Bocek M J, Saldivar J C, Swigut T, Cimprich K A. Transcription-replication conflict orientation modulates R-loop levels and activates distinct DNA damage responses. Cell. 2017; 170:774-786 e719.
44. Costantino L, Koshland D. Genome-wide map of R-loop-induced damage reveals how a subset of R-loops contributes to genomic instability. Mol. Cell. 2018; 71:487-497 e483.
45. Arora R, et al. RnaseH1 regulates TERRA-telomeric DNA hybrids and telomere maintenance in ALT tumour cells. Nat. Commun. 2014; 5:5220.
46. Boguslawski S J, et al. Characterization of monoclonal antibody to DNA. RNA and its application to immunodetection of hybrids. J Immunol. Methods. 1986; 89:123-130.
47. Cristini A, Groh M, Kristiansen M S, Gromak N. RNA/DNA hybrid interactome identifies DXH9 as a molecular player in transcriptional termination and R-loop-associated DNA damage. Cell Rep. 2018; 23:1891-1905.
48. Silva S, Camino L P, Aguilera A. Human mitochondrial degradosome prevents harmful mitochondrial R loops and mitochondrial genome instability. Proc. Natl Acad. Sci. USA. 2018; 115:11024-11029.
49. Chen J Y, Zhang X, Fu X D, Chen L. R-ChIP for genome-wide mapping of R-loops by using catalytically inactive RNASEH1. Nat. Protoc. 2019; 14:1661-1685.
50. Bazak L, Levanon E Y, Eisenberg E. Genome-wide analysis of Alu editability. Nucleic Acids Res. 2014; 42:6876-6884.
51. Rodic N, Bums K H. Long interspersed element-1 (LINE-1): passenger or driver in human neoplasms?PloS Genet. 2013; 9:e1003402.
52. Bass B L, Weintraub H. An unwinding activity that covalently modifies its double-stranded RNA substrate. Cell. 1988; 55:1089-1098.
53. Wagner R W, Smith J E, Cooperman B S, Nishikura K. A double-stranded RNA unwinding activity introduces structural alterations by means of adenosine to inosine conversions in mammalian cells and Xenopus eggs. Proc. Natl Acad. Sci. USA. 1989; 86:2647-2651.
54. Zheng Y, Lorenzo C, Beal P A. DNA editing in DNA/RNA hybrids by adenosine deaminases that act on RNA. Nucleic Acids Res. 2017; 45:3369-3377.
55. Eggington J M, Greene T, Bass B L. Predicting sites of ADAR editing in double-stranded RNA. Nat. Commun. 2011; 2:319.
56. Mergny J L, De Cian A, Ghelab A, Sacca B, Lacroix L. Kinetics of tetramolecular quadruplexes. Nucleic Acids Res. 2005; 33:81-94.
57. Feretzaki M, et al. RAD51-dependent recruitment of TERRA lncRNA to telomeres through R-loops. Nature. 2020; 587:303-308.
58. Valente L, Nishikura K. RNA binding-independent dimerization of adenosine deaminases acting on RNA and dominant negative effects of nonfunctional subunits on dimer functions. J Biol. Chem. 2007; 282:16054-16061.
59. Luke B, et al. The Rat1p 5′ to 3′ exonuclease degrades telomeric repeat-containing RNA and promotes telomere elongation in Saccharomyces cerevisiae. Mol. Cell. 2008; 32:465-477.
60. Teasley D C, et al. Flap endonuclease 1 limits telomere fragility on the leading strand. J. Biol. Chem. 2015; 290:15133-15145.
61. Balk B, et al. Telomeric RNA-DNA hybrids affect telomere-length dynamics and senescence. Nat. Struct. Mol. Biol. 2013; 20:1199-1205.
62. Wang I X, et al. RNA-DNA differences are generated in human cells within seconds after RNA exits polymerase II. Cell Rep. 2014; 6:906-915.
63. Lockhart A, et al. Rnase H1 and H2 are differentially regulated to process RNA-DNA hybrids. Cell Rep. 2019; 29:2890-2900 e2895.
64. Grudic A, et al. Replication protein A prevents accumulation of single-stranded telomeric DNA in cells that use alternative lengthening of telomeres. Nucleic Acids Res. 2007; 35:7267-7278.
65. Porro A, Feuerhahn S, Reichenbach P, Lingner J. Molecular dissection of telomeric repeat-containing RNA biogenesis unveils the presence of distinct and multiple regulatory pathways. Mol. Cell. Biol. 2010; 30:4808-4817.
66. Le P N, Maranon D G, Altina N H, Battaglia C L, Bailey S M. TERRA, hnRNP A1, and DNA-PKcs interactions at human telomeres. Front. Oncol. 2013; 3:91.
67. Sui J, Zhang S, Chen B P C. DNA-dependent protein kinase in telomere maintenance and protection. Cell. Mol. Biol. Lett. 2020; 25:2.
68. Ohle C, et al. Transient RNA-DNA hybrids are required for efficient double-strand break repair. Cell. 2016; 167:1001-1013 e1007.
69. Ota H, et al. ADAR1 forms a complex with Dicer to promote microRNA processing and RNA-induced gene silencing. Cell. 2013; 153:575-589.
70. Miyoshi H, Blomer U, Takahashi M, Gage F H, Verma I M. Development of a self-inactivating lentivirus vector. J Virol. 1998; 72:8150-8157.
71. Shiromoto Y, et al. GPAT2, a mitochondrial outer membrane protein, in piRNA biogenesis in germline stem cells. RNA. 2013; 19:803-810.
72. Kind B, et al. Altered spatio-temporal dynamics of Rnase H2 complex assembly at replication and repair sites in Aicardi-Goutieres syndrome. Hum. Mol. Genet. 2014; 23:5950-5960.
73. Figiel M, et al. The structural and biochemical characterization of human Rnase H2 complex reveals the molecular basis for substrate recognition and Aicardi-Goutieres syndrome defects. J Biol. Chem. 2011; 286:10540-10550.
74. Shiromoto, Y., Sakurai, M., Minakuchi, M., Ariyoshi, K. & Nishikura, K. ADAR1 RNA editing enzyme regulates R-loop formation and genome stability at telomeres in cancer cells. Nature communications 12, 1654, doi:10.1038/s41467-021-21921-x (2021).
75. Tikhmyanova, N. et al. Small molecule perturbation of the CAND1-Cullin1-ubiquitin cycle stabilizes p53 and triggers Epstein-Barr virus reactivation. PLoS Pathog 13, e1006517, doi:10.1371/journal.ppat.1006517 (2017).
76. Salvino, J. M. et al. Novel small molecule guanidine Sigma1 inhibitors for advanced prostate cancer. Bioorg Med Chem Lett 27, 2216-2220, doi:10.1016/j.bmcl.2017.03.030 (2017).
77. Herbert, A. ADAR and Immune Silencing in Cancer. Trends Cancer 5, 272-282, doi:10.1016/j.trecan.2019.03.004 (2019).
78. Nishikura, K. Functions and regulation of RNA editing by ADAR deaminases. Annual review of biochemistry 79, 321-349 (2010).
79. Sakurai, M. et al. ADAR1 controls apoptosis of stressed cells by inhibiting Staufen1-mediated mRNA decay. Nature structural & molecular biology 24, 534-543, doi:10.1038/nsmb.3403 (2017).
80. Barnoud, T. et al. A Novel Inhibitor of HSP70 Induces Mitochondrial Toxicity and Immune Cell Recruitment in Tumors. Cancer Res 80, 5270-5281, doi:10.1158/0008-5472.CAN-20-0397 (2020).
81. Sommer B, Kohler M, Sprengel R, Seeburg P H. RNA editing in brain controls a determinant of ion flow in glutamate-gated channels. Cell. 1991; 67:11-19. [PubMed: 1717158].
82. Lomeli H, et al. Control of kinetic properties of AMPA receptor channels by nuclear RNA editing. Science. 1994; 266:1709-1713. [PubMed: 7992055].
83. Sailer A, et al. Generation and analysis of GluR5(Q636R) kainate receptor mutant mice. J. Neurosci. 1999; 19:8757-8764. [PubMed: 10516295].
84. Egebjerg J, Heinemann S F. Ca2+ permeability of unedited and edited versions of the kainate selective glutamate receptor GluR6. Proc. Natl Acad. Sci. USA. 1993; 90:755-759. [PubMed: 7678465].
85. Kohler M, Burnashev N, Sakmann B, Seeburg P H. Determinants of Ca2+ permeability in both TM1 and TM2 of high affinity kainate receptor channels: diversity by RNA editing. Neuron. 1993; 10:491-500. [PubMed: 7681676].
86. Bums C M, et al. Regulation of serotonin-2C receptor G-protein coupling by RNA editing. Nature. 1997; 387:303-308. [PubMed: 9153397].
87. Bhalla T, Rosenthal J J, Holmgren M, Reenan R. Control of human potassium channel inactivation by editing of a small mRNA hairpin. Nat. Struct. Mol. Biol. 2004; 11:950-956. [PubMed: 15361858].
88. Daniel C, Wahlstedt H, Ohlson J, Bjork P, Ohman M. Adenosine-to-inosine RNA editing affects trafficking of the γ-aminobutyric acid type A (GABAA) receptor. J. Biol. Chem. 2011; 286:2031-2040. [PubMed: 21030585].
89. Levanon E Y, et al. Evolutionarily conserved human targets of adenosine to inosine RNA editing. Nucleic Acids Res. 2005; 33:1162-1168. [PubMed: 15731336].
90. Galeano F, et al. Human BLCAP transcript: new editing events in normal and cancerous tissues. Int. J. Cancer. 2010; 127:127-137. [PubMed: 19908260].
91. Chan T H, et al. A disrupted RNA editing balance mediated by ADARs (Adenosine DeAminases that act on RNA) in human hepatocellular carcinoma. Gut. 2014; 63:832-843. [PubMed: 23766440].
92. Martinez H D, et al. RNA editing of androgen receptor gene transcripts in prostate cancer cells. J. Biol. Chem. 2008; 283:29938-29949. [PubMed: 18708348].
93. Chen L, et al. Recoding RNA editing of AZIN1 predisposes to hepatocellular carcinoma.

Nat. Med. 2013; 19:209-216. [PubMed: 23291631].
94. Yeo J, Goodman R A, Schirle N T, David S S, Beal P A. RNA editing changes the lesion specificity for the DNA repair enzyme NEIL1. Proc. Natl Acad. Sci. USA. 2010; 107:20715-20719. [PubMed: 21068368].
95. Shimokawa T, et al. RNA editing of the GLI1 transcription factor modulates the output of Hedgehog signaling. RNA Biol. 2013; 10:321-333. [PubMed: 23324600].
96. Han S W, et al. RNA editing in RHOQ promotes invasion potential in colorectal cancer. J. Exp. Med. 2014; 211:613-621. [PubMed: 24663214].

1. A screening method comprising:
a) providing a test compound to a non-ALT cancer cell comprising a nucleic acid that comprises a dual reporter system, said dual reporter system comprising i) a first reporter, ii) an adenosine-to-inosine (A-to-I) editing site within a stop codon of a short hairpin dsRNA, and iii) a second reporter, wherein the first reporter and second reporter are different, and wherein adenosine to inosine editing alters the stop codon to Trp, resulting in in-frame translation of the second reporter,
b) measuring the levels of expression of the first reporter and the second reporter; wherein the ratio of the expression level of the second reporter to the ratio of the expression level of the first reporter demonstrates A-to-I editing efficiency;
wherein the test compound is an inhibitor of ADAR1 when the editing efficiency is decreased at least 60% as compared to a control.

2. The screening method according to claim 1, wherein editing efficiency is assessed about 1 day after providing the test compound.

3. The screening method according to claim 1, further comprising:
c) measuring the level of the first reporter at least 3 days after providing the test compound, wherein a reduction of the level of the first reporter of at least 30% as compared to a control identifies an ADAR1 inhibitor.

4. The screening method according to claim 3, wherein step c) is performed before step b).

5. The screening method according to claim 3, further comprising:
d) measuring the level of the first reporter about 1 day after providing the test compound, wherein a reduction of the level of the first reporter of less than about 40% as compared to a control identifies an ADAR1 inhibitor.

6. The screening method according to claim 1, wherein the cancer cell stably expresses the dual reporter system.

7. (canceled)

8. A method comprising methods b), c), and d) of claim 5, wherein b), c), and d) are performed and are performed in any order.

9. A composition comprising:
a cancer cell comprising a nucleic acid that comprises a dual reporter system, said dual reporter system comprising i) a first reporter, ii) an adenosine-to-inosine (A-to-I) editing site within a stop codon of a short hairpin dsRNA, and iii) a second reporter, wherein the first reporter and second reporter are different.

10. The composition according to claim 9, wherein the A-to-I editing site is at an A-C mismatched base pair.

11. The composition according to claim 9, wherein the cancer cell stably expresses the dual reporter system.

12. The composition according to claim 9, wherein the cancer cell is a non-ALT cancer cell.

13. A pharmaceutical composition comprising a pharmaceutically acceptable carrier and one or more of:
a) [[amino-[3-chloro-4-[(4-chlorophenyl)methoxy]phenyl]methylidene]amino]5-nitrofuran-2-carboxylate
b) 5,7-dimethyl-2-phenylpyrazolo[1,5-a]pyrimidine
c) 3-chloro-N-(4-cyanophenyl)-1-benzothiophene-2-carboxamide
d) 1-[(4-chlorophenyl)methylsulfanyl]-3-phenylpyrido[1,2-a]benzimidazole-4-carbonitrile
e) N-(1-benzylpiperidin-4-yl)-6-phenylthieno[3,2-d]pyrimidin-4-amine
f) 5-(4-chlorophenyl)-3-methylsulfanyl-1H-pyrazole-4-carbonitrile
g) 4-[6-(3,5-dimethylpyrazol-1-yl)pyridazin-3-yl]thiomorpholine
h) 3-(3,4,5-trimethoxyphenyl)-4,5-dihydro-1H-pyridazin-6-one;
i) 4-(furan-2-yl)-2,6-bis(methylsulfanyl)pyrimidine-5-carbonitrile,
or a prodrug, derivative, pharmaceutical salt, or analog thereof.

14. The pharmaceutical composition according to claim 13, further comprising a checkpoint inhibitor.

15. The pharmaceutical composition according to claim 14, wherein the checkpoint inhibitor is a PD-1 or PD-L1 inhibitor.

16. The pharmaceutical composition according to claim 13, further comprising a chemotherapeutic agent.

17. A method of treating cancer comprising administering the pharmaceutical composition of claim 13.

18. The method according to claim 17, wherein the method further includes administering a checkpoint inhibitor.

19. The method according to claim 18, wherein the cancer is a telomerase-reactivated cancer.

20. The method according to claim 18, wherein the cancer is a non-ALT cancer.

21-24. (canceled)
